(19) United States
(12) Patent Application Publication
(10) Pub. No.: US 2021/0165001 A1
(43) Pub. Date: Jun. 3, 2021

(54) COMPOSITIONS AND METHODS FOR DETECTION AND IMAGING OF AMYLOID FIBRILS, AMYLOID PLAQUES, RNA, AND NUCLEOLI

(71) Applicant: The University of Hong Kong, Hong Kong (CN)

(72) Inventors: Wing-Wah Vivian Yam, Hong Kong (CN); Sin-Yee Law, Hong Kong (CN); Ching-Lam Margaret Yeung, Hong Kong (CN)

(21) Appl. No.: 17/265,756

(22) PCT Filed: Aug. 2, 2019
(86) PCT No.: PCT/CN2019/099143

§ 371 Date: Feb. 3, 2021

Related U.S. Application Data
(60) Provisional application No. 62/714,157, filed on Aug. 3, 2018.
(60) Provisional application No. 62/854,929, filed on May 30, 2019.

(51) Int. Cl. G01N 33/68; G01N 33/58; C07D 487/22

(52) U.S. Cl. CPC G01N 33/6896; G01N 33/582; G01N 2800/2814; G01N 2500/04; C07D 487/22

(57) ABSTRACT

Compounds are used for detection and/or imaging of amyloid, plaque, or both of proteins or peptides, for screening or testing the efficacy of inhibitors against amyloidosis and/or fibrillar growth of proteins or peptides, and/or for detection of RNA and nucleolus imaging. The compounds are d8 or d10 metal complexes or salts thereof. The metal complexes of the compounds can bind to amyloid, plaque, or both, of the proteins or peptides and/or RNA, nucleolus, or both. The binding induces accumulation and supramolecular self-assembly of the metal complexes, thereby causing hanges in the photophysical properties of the metal complexes.

### FIELD OF THE INVENTION

The invention is generally directed to detecting and/or imaging analytes and more particularly to detecting and/or imaging amyloid, plaque, or both, of proteins or peptides. The invention is also in the field of screening or testing the efficacy of inhibitors of amyloid fibrillation and/or amyloid plaque formation, and the field of detecting and/or imaging amyloid fibrillation and plaque formation relevant to neurodegenerative diseases, dementia, and other associated diseases or disorders. The invention is also directed to detecting RNA and imaging nucleoli.

### BACKGROUND OF THE INVENTION

Amyloids are thread-like aggregates of proteins or peptides which are frequently ordered in a β-sheet conformation, allowing abnormal proteins or peptides to build up in tissues. They have been associated with many disorders with a multitude of disparate symptoms, including Alzheimer's disease, type 2 diabetes mellitus, Huntington's disease, Parkinson's disease, dementia, and other associated diseases or disorders. Collectively known as amyloidosis or proteopathy, these diseases or disorders have been identified to arise from the abnormal aggregation of a variety of proteins or peptides, and accordingly disrupt the function of tissues and/or organs.

Alzheimer's disease is one of the most common proteopathy-induced neurodegenerative disorders and the leading cause of dementia. It is believed that extracellular amyloid-β peptide formation and deposition is the primary event in the disease process, followed by hyperphosphorylation of tau proteins and formation of neurofibrillary tangles (Hamley, Chem. Rev., 112:5147-5192 (2012)). These may first lead to malfunctions in the biochemical communication of neuronal cells and subsequently result in neuronal cell death.

Parkinson's disease is another representative example of proteopathy-induced neurodegenerative disorders. The societal impact of Parkinson's disease grows as the size of the elderly population increases. In general, Parkinson's disease is associated with the abnormal accumulation of α-synuclein aggregates to form amyloid fibrils and the preferential death of dopamine-producing neurons in the midbrain (Singleton et al., Science, 302:841-841 (2003)). These lead to a drastic depletion of dopamine in the striatum. As Parkinson's disease mainly affects the motor system, the most obvious symptoms are balance disorder, bradykinesia, spasticity, and tremor.

Although amyloidosis and proteopathy constitute an important topic of biomedical research, especially neuroscience research, the early diagnosis of these disorders remains an unmet challenge. A number of methods are available for the detection of amyloid fibrillation, including colorimetric staining, fluorescent staining, and enzyme-linked immunosorbent assay (ELISA). However, these existing detection methods have drawbacks that limit their applications. For example, colorimetric staining using dyes such as Congo red often requires the use of polarized light microscopy, and the birefringence of the dyes is difficult to interpret (Biancalana et al., Biochim. Biophys. Acta, 1804:1405-1412 (2010)). Fluorescent staining using thioflavin T is widely used to identify and stain misfolded protein aggregates. However, the emission signal of thioflavin T is not in the red or near-infrared (NIR) region. Assessment of thioflavin T is thus complicated by autofluorescence from various biological molecules due to an overlap of the fluorescence emission spectra (Anderson et al., J. Clin. Pathol., 27:656-663 (1974)). Moreover, thioflavin T has an unfavorably small Stokes shift, further limiting the detection of amyloid and plaque of proteins or peptides. ELISA also has inherent drawbacks, as it requires the use of costly enzyme-linked antibodies and carcinogens during chemiluminescence detection (Yu et al., Angew. Chem., Int. Ed., 53:12832-12835 (2014)). It also bears the risk of underestimation or false-positive determination of amyloid levels (Stenh et al., Ann. Neurol., 58:147-150 (2005); Sehlin et al., J. Alzheimers Dis., 21:1295-1301 (2010)).

As a key component as well as the largest structure in the nucleus of eukaryotic cells, nucleolus is best known as the site of ribosome biogenesis (Olson et al., Trends Cell Biol., 10:189-196 (2000); Nemeth et al., Trends Genet., 27:149-156 (2011); O'Sullivan et al., Biomol. Concepts, 4:277-286 (2013)). Nucleolus participates in the transcription and processing of ribosomal RNA (rRNA) and plays a role in the assembly of ribosomal proteins. Aberrant morphological changes or alterations in the associated number of nucleoli can be a cause of particular types of cancers and other human disorders (Busch et al., Cancer Res., 23:313-339 (1963); Kelemen et al., Cancer, 65:1017-1020 (1990); Krystosek, Exp. Cell Res., 241:202-209 (1998); Derenzini et al., J. Pathol., 191:181-186 (2000); Lammerding et al., J. Cell Biol., 170:781-791 (2005); Quin et al., Biochim. Biophys. Acta, 1842:802-816 (2014); Woods et al., Biochim. Biophys. Acta, 1849:821-829 (2015)). As a result, the nucleolus has been regarded as a diagnostic biomarker for pathological detection of malignant lesions and is being investigated as a target for cancer chemotherapy. Ribonuclease (RNase) is a type of nuclease which catalyzes the degradation of RNA into smaller components (Raines, Chem. Rev., 98:1045-1066 (1998)). For example, pancreatic RNase, which is commonly abbreviated as RNase A, is the predominant endoribonuclease in human organs and tissues (Huang et al., PLoS One, 9:e96490 (2014)). It has been found to play a role in autoimmune disease, kidney failure, and pancreatic disease. An antitumor activity has also been reported as some members of the RNase A family have cytostatic and cytotoxic effects. They show differential cytotoxicity against tumor cells but not normal cells, since normal cells are protected due to their high affinity to RNase inhibitor (Gaur et al., J. Biol. Chem., 276:24978-24984 (2001)).

Although nucleolus plays a key role in disease theranostics, so far there is only one commercially available probe for nucleolus imaging, i.e., the SYTO™ RNASelect™ green fluorescent cell stain. It is virtually non-emissive in the absence of nucleic acid, but exhibits bright green fluorescence when bound to RNA (Yu et al., J. Mater. Chem. B, 4:2614-2619 (2016)). Despite being the only commercially available probe for nucleolus imaging, the SYTO™ RNASelect™ green fluorescent cell stain has a number of drawbacks, including high cost, poor photostability, small Stokes shift, and strict storage conditions. The photostability of biological probes is of crucial importance for the accuracy of organelle staining as well as the quality of confocal images. However, a major problem accompanying the use of most organic dyes or fluorophore molecules is photobleaching such that they permanently lose the ability to fluoresce (O'Mara et al., Talanta, 176:130-139 (2018)).

There is an urgent need to develop a method for rapid detection and/or imaging of amyloid, plaque, or both, of proteins or peptides, allowing for early diagnosis of diseases and disorders caused by amyloidosis and proteopathy. There is a tremendous demand to develop a method for quickly screening or evaluating the efficacy of inhibitors against amyloidosis and/or fibrillar growth of proteins or peptides. There is also an urgent need to develop a method for rapid detection of RNA and nucleolus imaging, allowing for early diagnosis of particular types of cancers and other human disorders.

It is the object of the present invention to provide compounds to detect and/or image an analyte or screen and/or test inhibitors, particularly to (1) detect and/or image amyloid, plaque, or both, of proteins or peptides, (2) screen or test the efficacy of inhibitors against amyloidosis and/or fibrillar growth of proteins or peptides, and/or (3) detect RNA and image nucleolus.

It is another object of the present invention to provide methods for detecting and/or imaging an analyte or screening and/or testing inhibitors, particularly for (1) detecting and/or imaging amyloid, plaque, or both, of proteins or peptides, (2) screening or testing the efficacy of inhibitors against amyloidosis and/or fibrillar growth of proteins or peptides, and/or (3) detecting RNA and imaging nucleolus.

It is yet another object of the present invention to provide kits for detecting and/or imaging an analyte or screening and/or testing inhibitors, particularly for (1) detecting and/or imaging amyloid, plaque, or both, of proteins or peptides, (2) screening or testing the efficacy of inhibitors against amyloidosis and/or fibrillar growth of proteins or peptides, and/or (3) detecting RNA and imaging nucleolus.

### BRIEF SUMMARY OF THE INVENTION

Disclosed are compounds, mixtures, compositions, kits, and methods for detecting and/or imaging an analyte or screening and/or testing inhibitors.

For example, in some forms, the compounds are d⁸ or d¹⁰ metal complexes or salts thereof, containing:

(a) a metal atom with a coordination number of 2, 3, or 4, selected from the group consisting of Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), and Hg(II); and

(b) one or more ligands with donor atoms independently selected from the group consisting of carbon (C), nitrogen (N), oxygen (O), phosphorus (P), sulfur (S), arsenic (As), and selenium (Se).

The metal complexes can have a planar structure or a partially planar structure. The metal complexes can bind to an analyte, wherein the binding of the metal complexes to the analyte induces aggregation and supramolecular self-assembly of the metal complexes through noncovalent metal-metal interactions. Noncovalent interactions such as π-π stacking interactions, electrostatic interactions, hydrogen bonding interactions, hydrophobic interactions, and combinations thereof can contribute to the binding of the metal complexes to the analyte, which leads to the aggregation and supramolecular self-assembly of the metal complexes.

In some forms, the analyte is amyloid, plaque, or both, of the proteins or peptides. In some forms, the analyte is RNA, nucleolus, or both.

The aggregation and supramolecular self-assembly of the metal complexes can create changes in the photophysical properties of the metal complexes. In some forms, the changes in the photophysical properties can include a change in optical absorbance, luminescence, resonance light scattering (RLS), or combinations thereof. In some forms, the change in luminescence can include an increase in the luminescence quantum yield and/or emission intensity. In some forms, the change in luminescence can be or include a shift, preferably a red-shift, of the emission energy or wavelength.

In some forms, the metal complexes bind to the analyte via noncovalent interactions, such as, but not limited to, π-π stacking interactions, electrostatic interactions, hydrogen bonding interactions, hydrophobic interactions, and combinations thereof. Then this metal complex-analyte ensemble allows the metal complexes to assemble in close proximity to form aggregates, thereby strengthening the noncovalent metal-metal interactions between molecules of the metal complexes and giving rise to changes in the photophysical properties, such as luminescence, of the metal complexes.

The specificity of the metal complexes to a given analyte is based on the combination of noncovalent interactions between them. As demonstrated by the description which follows and the examples, the noncovalent interactions between the metal complexes and the analyte can be designed via molecular engineering. The planar or partially planar structure of the d⁸ or d¹⁰ metal complexes allows the metal complexes to have a tendency towards the formation of highly ordered oligomeric structures. This feature can be adopted to the detection and/or imaging of a wide range of analytes. Based on the structural properties of both the analyte and the metal complexes, the possible noncovalent interactions between them can be predicted. As a result, the supramolecular self-assembly behaviors of the metal complexes towards the analyte can be estimated.

A d⁸ or d¹⁰ metal complex can be designed and/or engineered to bind an analyte of interest by selecting the metal center and/or the ligands of the metal center, particularly the functional groups on the ligands of the metal complexes. In some forms, the presence of a particular functional group on one or more ligands can give rise to or facilitate a specific interaction between the metal complex and the analyte of interest.

Preferably, the analyte has a repeating structure to allow for the aggregation and supramolecular self-assembly of the metal complexes on it. In some forms, the analyte is electrostatically attracted to the metal complexes, and electrostatic interactions between the analyte and the metal complexes can be one of the driving forces for binding. In some forms, the analyte is neutrally charged or electrostatically repulsive to the metal complexes, and the metal complexes can bind to such an analyte through other types of noncovalent interactions, such as, but not limited to, π-π stacking interactions, hydrogen bonding interactions, hydrophobic interactions, and combinations thereof.

The compounds can have a structure of Formula I:

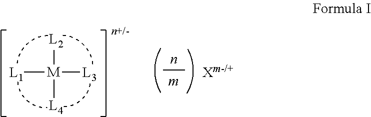

wherein

(a) M represents a metal atom selected from Pt(II), Pd(II), Ni(II), Ir(I), Rh(I), Au(III), Ag(III), and Cu(III),

(b) L₁, L₂, L₃, and L₄ represent ligands, wherein each ligand provides one donor atom to coordinate to the metal atom,

(c) n+/− represents the number of positive or negative charges carried by the metal complex in the formula, wherein n is zero or a positive integer, such as 1, 2, 3, 4, and 5,

(d) X^{m-/+} represents a counterion to maintain charge neutrality, wherein X^{m-/+} has a charge opposite to the charge of the metal complex and wherein m is zero or a positive integer, such as 1, 2, 3, 4, and 5, and wherein m=n or m≠n,

$$( e ) \frac{n}{m}$$

represents the stoichiometry of the counterion in the formula,

(f) dashed lines represent optional covalent linkages between the two ligands, optional fusion of ring moieties from the two ligands, or a combination thereof.

In some forms, L₁, L₂, and L₃ are optionally substituted, and/or optionally deprotonated C₆-C₅₀ arenes or C₃-C₅₀ heteroarenes, such as benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, and derivatives thereof.

The compounds can have a structure of Formula II:

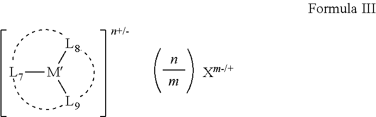

wherein M′ represents a metal atom selected from Ni(0), Pd(0), Pt(0), Cu(I), Ag(I), Au(I), Zn(II), Cd(II), and Hg(II),

wherein L₅ and L₆ represent ligands, wherein each ligand provides one donor atom to coordinate to the metal atom.

The compounds can also have a structure of Formula III:

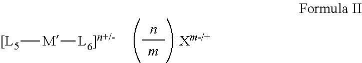

wherein L₇, L₈, and L₉ represent ligands, wherein each ligand provides one donor atom to coordinate to the metal atom.

Methods of making exemplary compounds are disclosed. The methods are compatible with a wide variety of functional groups, ligands, metal complexes, and compounds, and thus a wide variety of derivatives can be obtainable from the disclosed methods.

Methods of detecting amyloid, plaque, or both, of proteins or peptides in a sample containing the proteins or peptides are disclosed. The methods include (a) combining one or more of the disclosed compounds with the sample and (b) detecting changes in the photophysical properties of the metal complexes of the compounds. Detection of changes in the photophysical properties of the metal complexes indicates the presence of aggregation and supramolecular self-assembly of the metal complexes, wherein the presence of aggregation and supramolecular self-assembly of the metal complexes indicates the presence of amyloid, plaque, or both, of the proteins or peptides in the sample.

Methods of imaging amyloid, plaque, or both, of proteins or peptides in a sample containing the proteins or peptides are disclosed. The methods include (a) combining one or more of the disclosed compounds with the sample under conditions to allow for binding of the metal complexes of the compounds with the amyloid, plaque, or both, of the proteins or peptides and subsequent aggregation and supramolecular self-assembly of the metal complexes, wherein aggregation and supramolecular self-assembly of the metal complexes generates changes in the photophysical properties of the metal complexes of the compounds, and (b) imaging the amyloid, plaque, or both, of the proteins or peptides based on one or more photophysical properties that are specific for the metal complexes after aggregation and supramolecular self-assembly.

In some forms, the sample contains a human or non-human animal bodily fluid, a human or non-human animal tissue, or a combination thereof. The bodily fluid can be cerebrospinal fluid; the tissue can be brain tissue. In some forms, the amyloid, plaque, or both, of the proteins or peptides in the sample contains thread-like aggregates of the proteins or peptides, which are ordered in a β-sheet conformation.

Methods for testing the efficacy of inhibitors against amyloidosis and/or fibrillar growth of proteins or peptides are disclosed. The methods include (a) combining one or more of the disclosed compounds with an inhibitor-treated sample containing the proteins or peptides and, separately, with an untreated sample containing the proteins or peptides and (b) comparing the photophysical properties of the metal complexes of the compounds between the two samples. The magnitude of the difference in the photophysical properties of the metal complexes between the two samples indicates the extent of change in the state of aggregation and supramolecular self-assembly of the metal complexes; the extent of change in the state of aggregation and supramolecular self-assembly of the metal complexes indicates the efficacy of the inhibitors.

Methods of detecting RNA, nucleolus, or both in a sample are disclosed. The methods include (a) combining one or more of the disclosed compounds with the sample and (b) detecting changes in the photophysical properties of the metal complexes of the compounds. Detection of changes in the photophysical properties of the metal complexes indicates the presence of aggregation and supramolecular self-assembly of the metal complexes, wherein the presence of aggregation and supramolecular self-assembly of the metal complexes indicates the presence of RNA, nucleolus, or both, in the sample.

Methods of imaging nucleolus in a sample are disclosed. The methods include (a) combining one or more of the disclosed compounds with the sample under conditions to allow for binding of the metal complexes of the compounds with the nucleolus and subsequent aggregation and supramolecular self-assembly of the metal complexes, wherein aggregation and supramolecular self-assembly of the metal complexes generates changes in the photophysical properties of the metal complexes of the compounds, and (b) imaging the nucleolus based on one or more photophysical properties that are specific for the metal complexes after aggregation and supramolecular self-assembly.

In some forms, the sample contains eukaryotic cells. The cell can be, but not limited to, 3T3 cells, A549 cells, Chinese hamster ovary (CHO) cells, HEK 293 cells, HeLa cells, Hep G2 cells, and HT1080 cells.

Kits for use in detecting and/or imaging amyloid, plaque, or both, of proteins or peptides, for use in screening or testing the efficacy of inhibitors against amyloidosis and/or fibrillar growth of proteins or peptides, and/or for use in detecting RNA and imaging nucleolus are also disclosed. The kits can contain, in one or more containers, one or more of the disclosed compounds and optionally instructions for use. The kits can also contain a carrier.

Additional advantages of the disclosed compounds, mixtures, compositions, kits, and methods will be set forth in part in the description which follows, and in part will be understood from the description, or can be learned by practice of the disclosed compounds, mixtures, compositions, kits, and methods. The advantages of the disclosed compounds, mixtures, compositions, kits, and methods will be realized and attained by means of the elements and combinations particularly pointed out in the appended claims. It is to be understood that both the foregoing general description and the following detailed description are exemplary and explanatory only and are not restrictive of the invention as claimed. It is also understood that the disclosed compounds, mixtures, compositions, kits, and methods are not limited to the particular methodology, protocols, and/or reagents described as these can vary.

### BRIEF DESCRIPTION OF THE DRAWINGS

The accompanying drawings, which are incorporated in and constitute a part of this specification, illustrate several embodiments of the disclosed compounds, mixtures, compositions, kits, and methods, and together with the description, serve to explain the principles of the disclosed compounds, mixtures, compositions, kits, and methods.

FIG. 1 shows the UV-vis absorption spectra of complex 1-Pt in dimethylformamide (DMF) (line a) and aqueous (line b) solutions at 298 K.

FIG. 2 shows the normalized emission spectra of complex 1-Pt in DMF (line a) and aqueous (line b) solutions at 298 K.

FIG. 3A shows the UV-vis absorption spectra of complex 1-Pt (50 μM) upon addition of different amounts of insulin amyloid (0-10 μM) in PBS buffer. The arrows indicate the trends of spectral changes. FIG. 3B shows a plot of the absorbance at 550 nm versus the concentration of insulin amyloid.

FIG. 4A shows the corrected emission spectra of complex 1-Pt (50 μM) upon addition of different amounts of insulin amyloid (0-10 μM) in PBS buffer. The arrow indicates the trend of spectral changes. FIG. 4B shows a plot of the relative emission intensity at 650 nm versus the concentration of insulin amyloid.

FIG. 5A shows the RLS spectra of complex 1-Pt (50 μM) upon addition of different amounts of insulin amyloid (0-10 μM) in PBS buffer. The arrows indicate the trends of spectral changes. FIG. 5B shows a plot of the relative RLS intensity at 550 nm versus the concentration of insulin amyloid.

FIG. 6A shows the UV-vis absorption spectra of complex 1-Pt (50 μM) upon addition of different amounts of native insulin (0-10 μM) in PBS buffer. FIG. 6B shows a plot of the absorbance at 550 nm versus the concentration of native insulin.

FIG. 7A shows the corrected emission spectra of complex 1-Pt (50 μM) upon addition of different amounts of native insulin (0-10 μM) in PBS buffer. FIG. 7B shows a plot of the relative emission intensity at 650 nm versus the concentration of native insulin.

FIG. 8A shows the RLS spectra of complex 1-Pt (50 μM) upon addition of different amounts of native insulin (0-10 μM) in PBS buffer. FIG. 8B shows a plot of the relative RLS intensity at 550 nm versus the concentration of native insulin.

FIG. 9A shows the corrected emission spectra of thioflavin T (10 μM) upon addition of insulin samples (10 μM) with different incubation times. The arrow indicates the trend of spectral changes. FIG. 9B shows a plot of the relative emission intensity at 490 nm versus the incubation time.

FIG. 10A shows the UV-vis absorption spectra of complex 1-Pt (50 μM) upon addition of insulin samples (10 μM) with different incubation times. The arrows indicate the trends of spectral changes. FIG. 10B shows a plot of the absorbance at 550 nm versus the incubation time.

FIG. 11A shows the corrected emission spectra of complex 1-Pt (50 μM) upon addition of insulin samples (10 μM) with different incubation times. The arrow indicates the trend of spectral changes. FIG. 11B shows a plot of the relative emission intensity at 650 nm versus the incubation time.

FIG. 12A shows the RLS spectra of complex 1-Pt (50 μM) upon addition of insulin samples (10 μM) with different incubation times. The arrows indicate the trends of spectral changes. FIG. 12B shows a plot of the relative RLS intensity at 550 nm versus the incubation time.

FIG. 13 shows the schematic illustration of the design rationale for the luminescence turn-on assay for detection and/or imaging of amyloid fibrillation and plaque formation using a d⁸ or d¹⁰ metal complex.

FIG. 14A shows the corrected emission spectra of different amounts of complex 1-Pt (0-50 μM) upon addition of insulin amyloid (10 μM) in PBS buffer. The arrow indicates the trend of spectral changes. FIG. 14B shows a plot of the relative emission intensity at 650 nm versus the concentration of complex 1-Pt.

FIG. 15A shows the luminescence confocal image prepared from insulin amyloid (10 μM) stained with complex 1-Pt (50 μM) in PBS buffer. FIG. 15B shows the bright-field confocal image prepared from insulin amyloid (10 μM) stained with complex 1-Pt (50 μM) in PBS buffer. FIG. 15C shows the merged confocal image prepared from insulin amyloid (10 μM) stained with complex 1-Pt (50 μM) in PBS buffer.

FIG. 16 shows plots of the relative emission intensity at 490 nm versus the incubation time, for an ensemble of mixtures of thioflavin T (10 μM) and different insulin samples (10 μM). The insulin samples were incubated in the denaturing buffer solution in the presence of different concentrations of L-ascorbic acid (0 (▪), 10 (•), 20 (▴), 50 (▾), 70 ( [symbol] ), 100 ( [symbol] ) mM).

FIG. 17 shows plots of the relative emission intensity at 650 nm versus the incubation time, for an ensemble of mixtures of complex 1-Pt (50 μM) and different insulin samples (10 μM). The insulin samples were incubated in the denaturing buffer solution in the presence of different concentrations of L-ascorbic acid (0 (▪), 10 (═O), 20 (▴), 50 (V), 70 ( [symbol] ), 100 ( [symbol] ) mM).

FIG. 18 is a bar chart showing the relative emission intensity at 650 nm of an ensemble of mixtures containing complex 1-Pt (50 μM), insulin amyloid (10 μM), and different metal ions (100 μM) in PBS buffer. (A) No metal ion, (B) Mg²⁺, (C) Ca²⁺, (D) Mn²⁺, (E) Fe²⁺, (F) Fe³⁺, (G) Cu²⁺, and (H) Zn²⁺. The “Pt” group represents a negative control, which contains complex 1-Pt (50 μM) but no insulin amyloid.

FIG. 19A is a bar chart showing the relative emission intensity at 650 nm of a solution containing complex 1-Pt (50 μM) and different biomolecules in PBS buffer. (A) α-Amylase (10 μM), (B) albumin from bovine serum (10 μM), (C) albumin from human serum (10 μM), (D) alkaline phosphatase (10 μM), (E) trypsin (10 μM), (F) DNA (10 μg mL⁻¹), and (G) RNA (10 μg mL⁻¹). The “Pt” group represents a negative control, which contains complex 1-Pt (50 μM) but no insulin amyloid. The “Amyloid” group represents a positive control, which contains complex 1-Pt (50 μM) and insulin amyloid (10 μM). FIG. 19B is a bar chart showing the relative emission intensity at 650 nm of an ensemble of mixtures containing complex 1-Pt (50 μM), insulin amyloid (10 μM), and different biomolecules in PBS buffer. (A) α-Amylase (10 μM), (B) albumin from bovine serum (10 μM), (C) albumin from human serum (10 μM), (D) alkaline phosphatase (10 μM), (E) trypsin (10 μM), (F) DNA (10 μg mL⁻¹), and (G) RNA (10 μg mL⁻¹). The “Pt” group represents a negative control, which contains complex 1-Pt (50 μM) but no insulin amyloid. The “Amyloid” group represents a positive control, which contains complex 1-Pt (50 μM) and insulin amyloid (10 μM).

FIG. 20A is a bar chart showing the cell viability of HeLa cells after incubation with different concentrations of complex 1-Pt (0, 6.25, 12.5, 25, 50, 100 μM) at 37° C. for 24 hours. FIG. 20B is a bar chart showing the cell viability of CHO cells after incubation with different concentrations of complex 1-Pt (0, 6.25, 12.5, 25, 50, 100 μM) at 37° C. for 24 hours.

FIG. 21 shows the UV-vis absorption spectra of complex 2-Pt in aqueous solution at 298 K.

FIG. 22 shows the normalized emission spectra of complex 2-Pt in aqueous solution at 298 K.

FIG. 23A shows the UV-vis absorption spectra of complex 2-Pt (20 μM) upon addition of different amounts of RNA (0-10 μg mL⁻¹) in PBS buffer. The arrows indicate the trends of spectral changes. FIG. 23B shows a plot of the absorbance at 550 nm versus the concentration of RNA.

FIG. 24A shows the corrected emission spectra of complex 2-Pt (20 μM) upon addition of different amounts of RNA (0-10 μg mL⁻¹) in PBS buffer. The arrow indicates the trend of spectral changes. FIG. 24B shows a plot of the relative emission intensity at 670 nm versus the concentration of RNA.

FIG. 25A shows the RLS spectra of complex 2-Pt (20 μM) upon addition of different amounts of RNA (0-10 μg mL⁻¹) in PBS buffer. The arrows indicate the trends of spectral changes. FIG. 25B shows a plot of the relative RLS intensity at 550 nm versus the concentration of RNA.

FIG. 26 is a bar chart showing the zeta potential of complex 2-Pt (20 μM) upon addition of different amounts of RNA (0-10 μg mL⁻¹) in PBS buffer.

FIG. 27A shows the corrected emission spectra of different amounts of complex 2-Pt (0-20 μM) upon addition of RNA (10 μg mL⁻¹) in PBS buffer. The arrow indicates the trend of spectral changes. FIG. 27B shows a plot of the relative emission intensity at 670 nm versus the concentration of complex 2-Pt.

FIG. 28A shows the luminescence confocal image of fixed HeLa cells stained with complex 2-Pt (20 μM) at 37° C. for 1 hour. FIG. 28B shows the bright-field confocal image of fixed HeLa cells stained with complex 2-Pt (20 μM) at 37° C. for 1 hour. FIG. 28C shows the merged confocal image of fixed HeLa cells stained with complex 2-Pt (20 μM) at 37° C. for 1 hour.

FIG. 29A shows the luminescence confocal image of fixed CHO cells stained with complex 2-Pt (20 μM) at 37° C. for 1 hour. FIG. 29B shows the bright-field confocal image of fixed CHO cells stained with complex 2-Pt (20 μM) at 37° C. for 1 hour. FIG. 29C shows the merged confocal image of fixed CHO cells stained with complex 2-Pt (20 μM) at 37° C. for 1 hour.

FIG. 30 shows the schematic illustration of the design rationale for the luminescence turn-on assay for detection of RNA and nucleolus imaging using a d⁸ or d¹⁰ metal complex.

FIG. 31A shows the luminescence confocal image of a fixed HeLa cell stained with complex 2-Pt (20 μM) at 37° C. for 1 hour. FIG. 31B shows the relative emission intensity profile across the fixed HeLa cell from FIG. 31A. The x-axis represents the scanning distance.

FIG. 32A shows the luminescence confocal image of a fixed CHO cell stained with complex 2-Pt (20 μM) at 37° C. for 1 hour. FIG. 32B shows the relative emission intensity profile across the fixed CHO cell from FIG. 32A. The x-axis represents the scanning distance.

FIG. 33A shows the luminescence confocal image of fixed HeLa cells stained with complex 2-Pt (20 μM) at 37° C. for 1 hour, followed by incubation with RNase (30 μg mL⁻¹) at 37° C. for 2 hours. FIG. 33B shows the bright-field confocal image of fixed HeLa cells stained with complex 2-Pt (20 μM) at 37° C. for 1 hour, followed by incubation with RNase (30 μg mL⁻¹) at 37° C. for 2 hours. FIG. 33C shows the merged confocal image of fixed HeLa cells stained with complex 2-Pt (20 μM) at 37° C. for 1 hour, followed by incubation with RNase (30 μg mL⁻¹) at 37° C. for 2 hours. FIG. 33D shows the luminescence confocal image of fixed HeLa cells stained with complex 2-Pt (20 μM) at 37° C. for 1 hour, followed by incubation with DNase (30 g mL⁻¹) at 37° C. for 2 hours. FIG. 33E shows the bright-field confocal image of fixed HeLa cells stained with complex 2-Pt (20 μM) at 37° C. for 1 hour, followed by incubation with DNase (30 μg mL⁻¹) at 37° C. for 2 hours. FIG. 33F shows the merged confocal image of fixed HeLa cells stained with complex 2-Pt (20 μM) at 37° C. for 1 hour, followed by incubation with DNase (30 μg mL⁻¹) at 37° C. for 2 hours. FIG. 33G shows the luminescence confocal image of fixed HeLa cells stained with complex 2-Pt (20 μM) at 37° C. for 1 hour, followed by incubation with both RNase and DNase (30 μg mL⁻¹ each) at 37° C. for 2 hours. FIG. 33H shows the bright-field confocal image of fixed HeLa cells stained with complex 2-Pt (20 μM) at 37° C. for 1 hour, followed by incubation with both RNase and DNase (30 μg mL⁻¹ each) at 37° C. for 2 hours. FIG. 33I shows the merged confocal image of fixed HeLa cells stained with complex 2-Pt (20 μM) at 37° C. for 1 hour, followed by incubation with both RNase and DNase (30 μg mL⁻¹ each) at 37° C. for 2 hours.

FIG. 34A shows the luminescence confocal image of fixed CHO cells stained with complex 2-Pt (20 μM) at 37° C. for 1 hour, followed by incubation with RNase (30 μg mL⁻¹) at 37° C. for 2 hours. FIG. 34B shows the bright-field confocal image of fixed CHO cells stained with complex 2-Pt (20 μM) at 37° C. for 1 hour, followed by incubation with RNase (30 μg mL⁻¹) at 37° C. for 2 hours. FIG. 34C shows the merged confocal image of fixed CHO cells stained with complex 2-Pt (20 μM) at 37° C. for 1 hour, followed by incubation with RNase (30 μg mL⁻¹) at 37° C. for 2 hours. FIG. 34D shows the luminescence confocal image of fixed CHO cells stained with complex 2-Pt (20 μM) at 37° C. for 1 hour, followed by incubation with DNase (30 μg mL⁻¹) at 37° C. for 2 hours. FIG. 34E shows the bright-field confocal image of fixed CHO cells stained with complex 2-Pt (20 μM) at 37° C. for 1 hour, followed by incubation with DNase (30 μg mL⁻¹) at 37° C. for 2 hours. FIG. 34F shows the merged confocal image of fixed CHO cells stained with complex 2-Pt (20 μM) at 37° C. for 1 hour, followed by incubation with DNase (30 μg mL⁻¹) at 37° C. for 2 hours. FIG. 34G shows the luminescence confocal image of fixed CHO cells stained with complex 2-Pt (20 μM) at 37° C. for 1 hour, followed by incubation with both RNase and DNase (30 μg mL⁻¹ each) at 37° C. for 2 hours. FIG. 34H shows the bright-field confocal image of fixed CHO cells stained with complex 2-Pt (20 μM) at 37° C. for 1 hour, followed by incubation with both RNase and DNase (30 μg mL⁻¹ each) at 37° C. for 2 hours. FIG. 34 shows the merged confocal image of fixed CHO cells stained with complex 2-Pt (20 μM) at 37° C. for 1 hour, followed by incubation with both RNase and DNase (30 μg mL⁻¹ each) at 37° C. for 2 hours.

FIG. 35A shows the luminescence confocal image of fixed HeLa cells stained with complex 2-Pt (20 μM) at 37° C. for 1 hour, followed by incubation with SYTO™ RNASelect™ green fluorescent cell stain (500 nM) at 37° C. for 20 minutes, with emission collected at 620-720 nm. FIG. 35B shows the luminescence confocal image of fixed HeLa cells stained with complex 2-Pt (20 μM) at 37° C. for 1 hour, followed by incubation with SYTO™ RNASelect™ green fluorescent cell stain (500 nM) at 37° C. for 20 minutes, with emission collected at 505-555 nm. FIG. 35C shows the luminescence confocal image of fixed HeLa cells stained with complex 2-Pt (20 μM) at 37° C. for 1 hour, followed by incubation with SYTO™ RNASelect™ green fluorescent cell stain (500 nM) at 37° C. for 20 minutes, with emission collected at 620-720 nm and 505-555 nm.

FIG. 36A shows the luminescence confocal image of fixed CHO cells stained with complex 2-Pt (20 μM) at 37° C. for 1 hour, followed by incubation with SYTO™ RNASelect™ green fluorescent cell stain (500 nM) at 37° C. for 20 minutes, with emission collected at 620-720 nm. FIG. 36B shows the luminescence confocal image of fixed CHO cells stained with complex 2-Pt (20 μM) at 37° C. for 1 hour, followed by incubation with SYTO™ RNASelect™ green fluorescent cell stain (500 nM) at 37° C. for 20 minutes, with emission collected at 505-555 nm. FIG. 36C shows the luminescence confocal image of fixed CHO cells stained with complex 2-Pt (20 μM) at 37° C. for 1 hour, followed by incubation with SYTO™ RNASelect™ green fluorescent cell stain (500 nM) at 37° C. for 20 minutes, with emission collected at 620-720 nm and 505-555 nm.

FIG. 37A is a bar chart showing the cell viability of HeLa cells after incubation with different concentrations of complex 2-Pt (0, 1.25, 2.5, 5, 10, 20 μM) at 37° C. for 24 hours. FIG. 37B is a bar chart showing the cell viability of CHO cells after incubation with different concentrations of complex 2-Pt (0, 1.25, 2.5, 5, 10, 20 μM) at 37° C. for 24 hours.

### DETAILED DESCRIPTION OF THE INVENTION

Disclosed are compounds, mixtures, compositions, and kits for detecting and/or imaging an analyte or screening and/or testing inhibitors, particularly for (1) detecting and/or imaging amyloid, plaque, or both, of proteins or peptides, (2) screening or testing the efficacy of inhibitors against amyloidosis and/or fibrillar growth of proteins or peptides, and/or (3) detecting RNA and imaging nucleolus.

In some forms, the compounds contain a d⁸ or d¹⁰ metal complex that can bind to an analyte. The analyte can be amyloid, plaque, or both, of the protein or peptide. The analyte can also be RNA, nucleolus, or both. The binding can produce a luminescence signal in the red to near-infrared (NIR) region via the aggregation and supramolecular self-assembly of the metal complex through noncovalent metal-metal interactions. Noncovalent interactions such as π-π stacking interactions, electrostatic interactions, hydrogen bonding interactions, hydrophobic interactions, and combinations thereof can contribute to the binding of the metal complexes to the analyte, which leads to the aggregation and supramolecular self-assembly of the metal complexes. Accompanied by visible light excitation and a large Stokes shift, interference associated with autofluorescence commonly encountered in the presence of various biological substrates can be reduced, rendering the compounds suitable for bioassays.

The disclosed compounds, mixtures, compositions, kits, and methods can be understood more readily by reference to the following detailed description of particular embodiments and the examples included therein and to the drawings and their previous and following description. All methods described herein can be performed in any suitable order unless otherwise indicated or otherwise clearly contradicted by context.

The use of any and all examples or exemplary language (e.g., “such as”) provided herein, is intended merely to better illustrate the invention and does not pose a limitation on the scope of the invention unless otherwise claimed. No language in the specification should be construed as indicating any non-claimed element as essential to the practice of the invention.

The disclosed compounds, mixtures, compositions, and kits, can be used for, can be used in conjunction with, can be used in preparation for, or are products of the disclosed methods. It is understood that when combinations, subsets, interactions, groups, etc. of these compounds, mixtures, compositions, and kits are disclosed, while specific reference of each various individual and collective combinations of these materials may not be explicitly disclosed, each is specifically contemplated and described herein. For example, if a compound is disclosed and discussed and a number of modifications that can be made to a number of molecules including the compound are discussed, each and every combination and permutation of the compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the compounds, mixtures, compositions, kits, components, etc. contemplated and disclosed as above can also be specifically and independently included or excluded from any group, sub-group, list, set, etc. of such materials. These concepts apply to all aspects of this application including, but not limited to, steps in methods of making and using the disclosed compounds, compositions, mixtures, and kits. Thus, if there are a variety of additional steps that can be performed it is understood that each of these additional steps can be performed with any specific embodiment or combination of embodiments of the disclosed methods, and that each such combination is specifically contemplated and should be considered disclosed.

Throughout the description and claims of this specification, the word “comprise” and variations of the word, such as “comprising” and “comprises,” mean “including but not limited to,” and are not intended to exclude, for example, other additives, components, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### I. DEFINITIONS

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “may,” “may be,” “can,” and “can be,” and related terms are intended to convey that the subject matter involved is optional (that is, the subject matter is present in some embodiments and is not present in other embodiments), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

Use of the term “about” is intended to describe values either above or below the stated value in a range of approx.+/−10%; in other embodiments the values may range in value either above or below the stated value in a range of approx.+/−5%; in other embodiments the values may range in value either above or below the stated value in a range of approx.+/−2%; in other embodiments the values may range in value either above or below the stated value in a range of approx.+/−1%. The preceding ranges are intended to be made clear by context, and no further limitation is implied.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers. The foregoing applies regardless of whether in particular cases some or all of these embodiments are explicitly disclosed.

A carbon range (e.g., C₁-C₁₀), is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed therein, such as C₂-C₉, C₃-C₈, C₁-C₅, etc.

The terms “derivative” and “derivatives” refer to chemical compounds/moieties with a structure similar to that of a parent compound/moiety but different from it in respect to one or more components, functional groups, atoms, etc. The derivatives can be formed from the parent compound/moiety by chemical reaction(s). The differences between the derivatives and the parent compound/moiety can include, but are not limited to, replacement of one or more functional groups with one or more different functional groups or introducing or removing one or more substituents of the hydrogen atoms. The derivatives can also differ from the parent compound/moiety with respect to the protonation state.

“Halogen” or “halide,” as used herein, refers to fluorine (F), chlorine (Cl), bromine (Br), iodine (I), or astatine (At).

The term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, a cyclic C₁-C₃₀ alkyl, a linear or branched C₁-C₃₀ alkyl, a linear or cyclic C₁-C₃₀ alkyl, a branched or cyclic C₁-C₃₀ alkyl, or a linear, branched, or cyclic C₁-C₃₀ alkyl.

The term “heteroalkyl” refers to alkyl groups where one or more carbon atoms are replaced with a heteroatom, such as, O, N, or S. Heteroalkyl groups can be linear, branched, or cyclic (either monocyclic or polycyclic). Preferred heteroalkyl groups have one to 30 carbon atoms, i.e., C₁-C₃₀ heteroalkyl. In some forms, a C₁-C₃₀ heteroalkyl can be a linear C₁-C₃₀ heteroalkyl, a branched C₁-C₃₀ heteroalkyl, a cyclic C₁-C₃₀ heteroalkyl, a linear or branched C₁-C₃₀ heteroalkyl, a linear or cyclic C₁-C₃₀ heteroalkyl, a branched or cyclic C₁-C₃₀ heteroalkyl, or a linear, branched, or cyclic C₁-C₃₀ heteroalkyl.

The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic (either monocyclic or polycyclic). Preferred alkenyl groups have two to 30 carbon atoms, i.e., C₂-C₃₀ alkenyl. In some forms, a C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl.

The term “heteroalkenyl” refers to alkenyl groups in which one or more doubly bonded carbon atoms are replaced by a heteroatom. Heteroalkenyl groups can be linear, branched, or cyclic (either monocyclic or polycyclic). Preferred heteroalkenyl groups have one to 30 carbon atoms, i.e., C₁-C₃₀ heteroalkenyl. In some forms, a C₁-C₃₀ heteroalkenyl can be a linear C₁-C₃₀ heteroalkenyl, a branched C₁-C₃₀ heteroalkenyl, a cyclic C₁-C₃₀ heteroalkenyl, a linear or branched C₁-C₃₀ heteroalkenyl, a linear or cyclic C₁-C₃₀ heteroalkenyl, a branched or cyclic C₁-C₃₀ heteroalkenyl, or a linear, branched, or cyclic C₁-C₃₀ heteroalkenyl.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic (either monocyclic or polycyclic). Preferred alkynyl groups have two to 30 carbon atoms, i.e., C₂-C₃₀ alkynyl. In some forms, a C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl.

The term “heteroalkynyl” refers to alkynyl groups in which one or more triply bonded carbon atoms are replaced by a heteroatom. Heteroalkynyl groups can be linear, branched, or cyclic (either monocyclic or polycyclic). Preferred heteroalkynyl groups have one to 30 carbon atoms, i.e., C₁-C₃₀ heteroalkynyl. In some forms, a C₁-C₃₀ heteroalkynyl can be a linear C₁-C₃₀ heteroalkynyl, a branched C₁-C₃₀ heteroalkynyl, a cyclic C₁-C₃₀ heteroalkynyl, a linear or branched C₁-C₃₀ heteroalkynyl, a linear or cyclic C₁-C₃₀ heteroalkynyl, a branched or cyclic C₁-C₃₀ heteroalkynyl, or a linear, branched, or cyclic C₁-C₃₀ heteroalkynyl.

The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner or can be fused. Preferred arenes have six to 50 carbon atoms, i.e., C₆-C₅₀ arenes. In some forms, a C₆-C₅₀ arenes can be a branched C₆-C₅₀ arenes, a monocyclic C₆-C₅₀ arenes, a polycyclic C₆-C₅₀ arenes, a branched polycyclic C₆-C₅₀ arenes, a fused polycyclic C₆-C₅₀ arenes, or a branched fused polycyclic C₆-C₅₀ arenes. Accordingly, in polycyclic aryl groups, the rings can be attached together in a pendant manner or can be fused. Preferred aryl groups have six to 50 carbon atoms, i.e., C₆-C₅₀ aryl. In some forms, a C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). Heteroarenes can be monocyclic or polycyclic. In polycyclic heteroarenes, the rings can be attached together in a pendant manner or can be fused. Preferred heteroarenes have three to 50 carbon atoms, i.e., C₃-C₅₀ heteroarenes. In some forms, a C₃-C₅₀ heteroarenes can be a branched C₃-C₅₀ heteroarenes, a monocyclic C₃-C₅₀ heteroarenes, a polycyclic C₃-C₅₀ heteroarenes, a branched polycyclic C₃-C₅₀ heteroarenes, a fused polycyclic C₃-C₅₀ heteroarenes, or a branched fused polycyclic C₃-C₅₀ heteroarenes. Accordingly, in polycyclic heteroaryl groups, the rings can be attached together in a pendant manner or can be fused. Preferred heteroaryl groups have three to 50 carbon atoms, i.e., C₃-C₅₀ heteroaryl. In some forms, a C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl.

The term “arylene” refers to divalent groups derived from arenes by removal of a hydrogen atom from two ring carbon atoms. In polycyclic arylene groups, the rings can be attached together in a pendant manner or can be fused. Preferred arylene groups have six to 50 carbon atoms, i.e., C₆-C₅₀ arylene. In some forms, a C₆-C₅₀ arylene can be a branched C₆-C₅₀ arylene, a monocyclic C₆-C₅₀ arylene, a polycyclic C₆-C₅₀ arylene, a branched polycyclic C₆-C₅₀ arylene, a fused polycyclic C₆-C₅₀ arylene, or a branched fused polycyclic C₆-C₅₀ arylene.

The term “heteroarylene” refers to divalent groups derived from heteroarenes by removal of a hydrogen atom from two ring atoms. In polycyclic heteroarylene groups, the rings can be attached together in a pendant manner or can be fused. Preferred heteroarylene groups have three to 50 carbon atoms, i.e., C₃-C₅₀ heteroalkenyl. In some forms, a C₃-C₅₀ heteroarylene can be a branched C₃-C₅₀ heteroarylene, a monocyclic C₃-C₅₀ heteroarylene, a polycyclic C₃-C₅₀ heteroarylene, a branched polycyclic C₃-C₅₀ heteroarylene, a fused polycyclic C₃-C₅₀ heteroarylene, or a branched fused polycyclic C₃-C₅₀ heteroarylene.

The term “aminooxy” refers to —O—NH₂, wherein the hydrogen atoms can be substituted with substituents.

The term “hydroxyamino” refers to —NH—OH, wherein the hydrogen atoms can be substituted with substituents.

The term “hydroxamate” refers to —C(═O)NH—OH, wherein the hydrogen atoms can be substituted with substituents.

The term “conjugated system” refers to a molecular entity whose structure can be represented as a system of alternating single and multiple bonds, e.g., CH₂═CH—CH═CH₂, CH₂═CH—C≡N. In such systems, conjugation is the interaction of one p-orbital with another across an intervening σ-bond in such structures. Conjugated systems can be or contain arene and/or heteroarene moieties.

The term “substituted,” as used herein, means that the chemical group or moiety contains one or more substituents replacing the hydrogen atoms in the chemical group or moiety. The substituents include, but not limited to:

a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHC₂, —CH₂NH₂, —NHCOH, —CHO, —COCl, —COF, —COBr, —COOH, —S₃H, —CH₂SO₂CH₃, —PO₃H₂, —OPO₃H₂, —P(═O)(OR^{G1′})(OR^{G2′}), —OP(═O)(OR^{G1′})(OR^{G2′}), —BR^{G1′}(OR^{G2′}), —B(OR^{G1′})(OR^{G2′}), or —G′R^{G1′} in which -G′ is —O—, —S—, —NR^{G2′}—, —C(═O)—, —S(═O)—, —SO₂—, —C(═O)O—, —C(═O)NR^{G2′}—, —OC(═O)—, —NR^{G2′}C(O)—, —OC(O)O—, —OC(═O)NR^{G2′}—, —NR^{G2′}C(═O)O—, —NR^{G2′}C(═O)NR^{G3′}—, —C(═S)—, —C(═S)S—, —SC(═S)—, —SC(═S)S—, —C(═NR^{G2′})—, —C(═NR^{G2′})O—, —C(═NR^{G2′})R^{G3′}—, —OC(═NR^{G2′})—, —NR^{G2′}C(═NR^{G3′})—, —NR^{G2′}SO₂—, —C(═NR^{G2′})NR^{G3′}—, —OC(═NR^{G2′})—, —NR^{G2′}C(═NR^{G3′})—, —NR^{G2′}SO₂—, —NR^{G2′}SO₂NR^{G3′}—, —NR^{G2′}C(═S)—, —SC(═S)NR^{G2′}—, —NR^{G2′}C(═S)S—, —NR^{G2′}C(═S)NR^{G3′}—, —SC(═NR^{G2′})—, —C(═S)NR^{G2′}—, —OC(═S)NR^{G2′}—, —NR^{G2′}C(═S)O—, —SC(═O)NR^{G2′}—, —NR^{G2′}C(O)S—, —C(O)S—, —SC(O)—, —SC(O)S—, —C(═S)O—, —OC(═S)—, —OC(═S)O—, —SO₂NR^{G2′}—, —BR^{G2′}—, or —PR^{G2′}—,

wherein each occurrence of R^{G1′}, R^{G2′}, and R^{G3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

The terms “d⁸ or d¹⁰ metal complex” and “d⁸ or d¹⁰ metal complexes” refer to any metal complex containing at least one metal atom with a d⁸ or d¹⁰ electronic configuration. The term “d⁸ or d¹⁰ metal complex aggregate” refers to local concentration enrichment of the d⁸ or d¹⁰ metal complex in the vicinity of an analyte. The analyte can be amyloid, plaque, or both, of proteins or peptides. The analyte can also be RNA, nucleolus, or both. The local concentration enrichment can be caused by noncovalent metal-metal interactions between molecules of the d⁸ or d¹⁰ metal complex. Noncovalent interactions, such as π-π stacking interactions, electrostatic interactions, hydrogen bonding interactions, and hydrophobic interactions, and combinations thereof can contribute to the binding between the analyte and the d⁸ or d¹⁰ metal complex and between different molecules of the d⁸ or d¹⁰ metal complex. In some forms, the d⁸ or d¹⁰ metal complex aggregate can be formed via aggregation and supramolecular self-assembly of the d⁸ or d¹⁰ metal complex after binding to the analyte.

The terms “ligand” and “ligands” refer to ions or molecules that bind to a central metal atom via one or more donor atoms, thereby forming a metal complex. The nature of metal ligand bonding can range from covalent to ionic. The metal ligand bond order can range from one to three. The bonding with the metal atom generally involves formal donation of one or more electron pairs from the donor atoms. The donor atoms can be carbon (C), nitrogen (N), oxygen (O), phosphorus (P), sulfur (S), arsenic (As), and selenium (Se).

The term “coordination number” refers to the total number of donor atoms coordinating to a central metal atom in a metal complex.

The terms “amyloid” and “plaque” employed herein can be thread-like aggregates of any proteins or peptides. The aggregates can be ordered in a β-sheet conformation; they can be distributed in solution or immobilized onto the surface of a solid support. The term “plaque” also refers to fibrous deposits of proteins, peptides, or amyloids.

The term “RNA” employed herein refers to ribonucleic acid, which is a polymeric molecule essential in various biological roles in coding, decoding, regulation, and expression of genes. It can be distributed in solution, located in a cell organelle such as nucleolus, or immobilized onto the surface of a solid support.

The term “nucleolus” employed herein refers to the largest structure in the nucleus of eukaryotic cells. Nucleoli are made of proteins, DNA, and RNA. They are widely known to serve as the site where ribosomal RNA (rRNA) is synthesized and processed.

The term “luminescence” refers to emission of light by a substance not resulting from heat. It can be caused by chemical reactions, electrical energy, subatomic motions or stress on a crystal, which all are ultimately caused by spontaneous emission. It can refer to chemiluminescence, i.e., the emission of light as a result of a chemical reaction. It can also refer to photoluminescence, i.e., the emission of light as a result of absorption of photons. The photoluminescence includes fluorescence and phosphorescence.

The terms “carrier” and “carriers” refer to all components present in a formulation or composition other than the active ingredient or ingredients. They can include but are not limited to, diluents, binders, lubricants, desintegrators, fillers, plasticizers, pigments, colorants, stabilizing agents, and glidants.

As used herein, “subject” includes, but is not limited to, human or non-human mammals. The term does not denote a particular age or sex. Thus, adult and newborn subjects, as well as fetuses, whether male or female, are intended to be covered. A patient refers to a subject afflicted with a disease or disorder. The term “patient” includes human and non-human mammal subjects.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### II. COMPOUNDS

Disclosed herein are compounds useful for detecting and/or imaging an analyte or screening and/or testing inhibitors.

In some forms, the analyte is amyloid, plaque, or both, of proteins or peptides. In some forms, the analyte is RNA, nucleolus, or both. The compounds can be used for screening or testing the efficacy of inhibitors against amyloidosis and/or fibrillar growth of proteins or peptides. The compounds can be also used for nucleolus imaging.

For example, in some forms, the compounds are d⁸ or d¹⁰ metal complexes or salts thereof, containing:

(a) a metal atom with a coordination number of 2, 3, or 4, selected from the group consisting of Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), and Hg(II); and

(b) one or more ligands with donor atoms independently selected from the group consisting of carbon (C), nitrogen (N), oxygen (O), phosphorus (P), sulfur (S), arsenic (As), and selenium (Se).

In some forms, the metal atom is not Au(III).

In some forms, the ligands do not have the following structures:

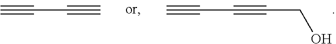

In some forms, the metal atom is Pt(II), and the ligand does not have the following structures:

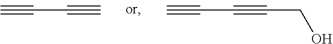

1. Metal Complexes

The metal complexes of the compounds can have a planar structure or a partially planar structure. Notably, square-planar d⁸ or d¹⁰ metal complexes have a tendency towards the formation of highly ordered extended linear chains or oligomeric structures in the solid state.

The metal complexes can bind to the analyte, wherein the binding of the metal complexes to the analyte induces aggregation and supramolecular self-assembly of the metal complexes through noncovalent metal-metal interactions. Noncovalent interactions such as π-π stacking interactions, electrostatic interactions, hydrogen bonding interactions, hydrophobic interactions, and combinations thereof can contribute to the binding of the metal complexes to the analyte, which leads to the aggregation and supramolecular self-assembly of the metal complexes. As a result, aggregates of the metal complex can be formed.

In some forms, the metal complexes can bind to amyloid, plaque, or both, of one or more proteins or peptides, including but not limited to, amyloid-β peptide, α-synuclein, insulin, huntingtin, tau protein, hyperphosphorylated tau protein (pTau), prion protein, IAPP (amylin), calcitonin, PrP^{Sc}, atrial natriuretic factor, apolipoprotein A1, serum amyloid A, medin, prolactin, transthyretin, lysozyme, beta-2 microglobulin, gelsolin, karatoepithelin, crystallin, desmin, selenoproteins, actin, cystatin, immunoglobulin light chain AL, S-IBM, and myosin. In some forms, the metal complexes can bind to amyloid, plaque, or both, of amyloid-β peptide.

In some forms, the metal complexes cannot bind or form self-assembled aggregates on native proteins or peptides. Preferably, the metal complexes cannot form self-assembled aggregates through noncovalent metal-metal interactions on native proteins or peptides.

In some forms, the metal complexes can bind to one or more types of RNA, including but not limited to, messenger RNA (mRNA), transfer RNA (tRNA), ribosomal RNA (rRNA), transfer-messenger RNA (tmRNA), antisense RNA (asRNA), enhancer RNA (eRNA), guide RNA (gRNA), ribozyme, short hairpin RNA (shRNA), small temporal RNA (stRNA), small interfering RNA (siRNA), and trans-acting siRNA (ta-siRNA).

In some forms, the metal complexes cannot bind or form self-assembled aggregates on double-stranded DNA. Preferably, the metal complexes cannot form self-assembled aggregates through noncovalent metal-metal interactions on double-stranded DNA. In some forms, the metal complexes can bind double-stranded DNA through noncovalent interactions such as T-T stacking interactions, electrostatic interactions, hydrogen bonding interactions, or combinations thereof, however, the aggregation or self-assembly of the metal complexes cannot be induced because of the double-stranded structure of DNA. The metal complexes can undergo intercalation such that they are inserted between the base pairs of DNA, making them unable to aggregate or self-assemble through noncovalent metal-metal interactions.

The aggregation and supramolecular self-assembly of the metal complexes can create changes in the photophysical properties of the metal complexes. In some forms, the changes in the photophysical properties can include a change in optical absorbance, luminescence, resonance light scattering (RLS), or combinations thereof.

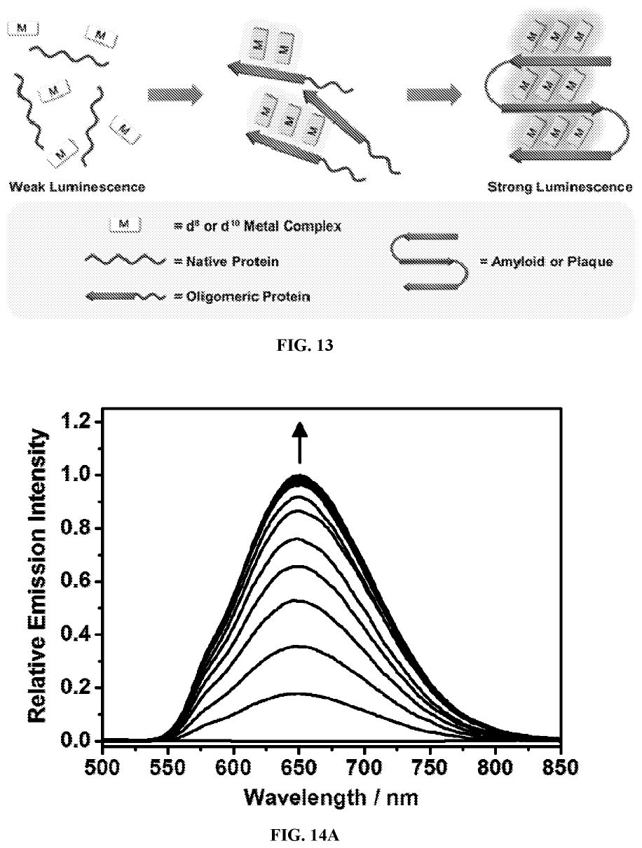

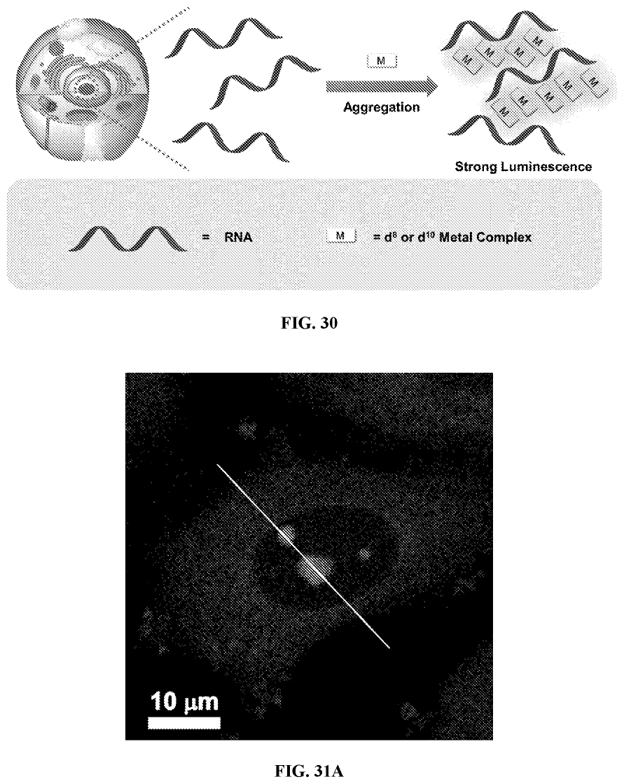

In some forms, the change in luminescence can be or include an increase in the luminescence quantum yield and/or emission intensity as illustrated in FIGS. 13 and 30. In some forms, the change in luminescence can be or include a shift, preferably a red-shift, of the emission energy or wavelength, compared to the non-aggregated or non-supramolecular self-assembled form. In some forms, the increase in the luminescence quantum yield and/or emission intensity and/or the shift in emission energy or wavelength can be caused by aggregation and supramolecular self-assembly of the metal complexes via noncovalent metal-metal interactions, similar to the effect of excitonic coupling. Noncovalent interactions such as π-π stacking interactions, electrostatic interactions, hydrogen bonding interactions, hydrophobic interactions, and combinations thereof can contribute to the binding of the metal complexes to the analyte, which leads to the aggregation and supramolecular self-assembly of the metal complexes. The increase in the luminescence quantum yield and/or emission intensity can be associated with a luminescence signal in the red to near-infrared (NIR) region, such as between about 600 nm and about 1000 nm. Preferably, the luminescence signal is associated with a large Stokes shift. In some forms, the Stokes shift is larger than 100 nm, larger than 150 nm, larger than 200 nm, larger than 250 nm, larger than 300 nm, larger than 350 nm, or larger than 400 nm. More preferably, the Stokes shift is larger than 400 nm. The change in luminescence can be or include a shift, preferably a red-shift, of the emission energy or wavelength, compared to the non-aggregated or non-supramolecular self-assembled form. The luminescence signal can originate from a transition between a singlet excited state and a singlet ground state or between a triplet excited state and a singlet ground state.

In some forms, the change in RLS can be or include an increase in the RLS signal intensity.

In some forms, the metal complexes bind to the analyte via noncovalent interactions, such as, but not limited to, π-π stacking interactions, electrostatic interactions, hydrogen bonding interactions, hydrophobic interactions, and combinations thereof. Then this metal complex-analyte ensemble allows the metal complexes to assemble in close proximity to form aggregates, thereby strengthening the noncovalent metal-metal interactions between molecules of the metal complexes and giving rise to changes in the photophysical properties, such as luminescence, of the metal complexes.

The specificity of the metal complexes to a given analyte is based on the combination of noncovalent interactions between them. As demonstrated by the description which follows and the examples, the noncovalent interactions between the metal complexes and the analyte can be designed via molecular engineering. The planar or partially planar structure of the d⁸ or d¹⁰ metal complexes allows the metal complexes to have a tendency towards the formation of highly ordered oligomeric structures. This feature can be adopted to the detection and/or imaging of a wide range of analytes. Based on the structural properties of both the analyte and the metal complexes, a person with ordinary skill in the art can predict the possible noncovalent interactions between them. As a result, the supramolecular self-assembly behaviors of the metal complexes towards the analyte can be estimated.

A d⁸ or d¹⁰ metal complex can be designed and/or engineered to bind an analyte of interest by selecting the metal center and/or the ligands of the metal center, particularly the functional groups on the ligands of the metal complexes. In some forms, the presence of a particular functional group on one or more ligands can give rise to or facilitate a specific interaction between the metal complex and the analyte of interest.

Preferably, the analyte has a repeating structure to allow for the aggregation and supramolecular self-assembly of the metal complexes on it. In some forms, the analyte is electrostatically attracted to the metal complexes, and electrostatic interactions between the analyte and the metal complexes can be one of the driving forces for binding. In some forms, the analyte is neutrally charged or electrostatically repulsive to the metal complexes, and the metal complexes can bind to such an analyte through other types of noncovalent interactions, such as, but not limited to, π-π stacking interactions, hydrogen bonding interactions, hydrophobic interactions, and combinations thereof.

2. Ligands of the Metal Complexes

The bonding between the ligands to the metal atoms in the metal complexes generally involves formal donation of one or more electron pairs from the donor atoms of the ligands. The donor atoms can be carbon (C), nitrogen (N), oxygen (O), phosphorus (P), sulfur (S), arsenic (As), and selenium (Se).

Exemplary ligands include optionally substituted C₆-C₅₀ arenes or C₃-C₅₀ heteroarenes, such as benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, and derivatives thereof.

Exemplary ligands also include halide ions, SCN⁻ (donor atom: S), O—NO₂⁻ (donor atom: O), N₃⁻, O₂⁻, S₂⁻, H₂O, O—NO⁻ (donor atom: O), NCS⁻ (donor atom: N), NH₃, NO₂⁻ (donor atom: N), N≡C⁻ (donor atom: N), C≡N⁻ (donor atom: C), CO (donor atom: C), C≡C—R⁻, OR⁻, SR⁻, SeR⁻, SeR¹R², N₃R, N≡C—R (donor atom: N), NR¹R²R³, PR¹R²R³, and AsR¹R²R³. In some forms, one or more ligands of the metal complexes are C≡C—R⁻.

In some forms of these ligands, R, R¹, R², and R³ are independently:

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,

In some forms, R, R¹, R², R³, and their organic substituents are optionally and independently substituted with one or more groups, wherein each such group is independently:

a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —S₃H, —CH₂SO₂CH₃, —PO₃H₂, —OPO₃H₂, —P(═O)(OR^{G1′})(OR^{G2′}), —OP(═O)(OR^{G1′})(OR^{G2′}), —BR^{G1′}(OR^{G2′}), —B(OR^{G1′})(OR^{G2′}), or -G′R^{G1′} in which -G′ is —O—, —S—, —NR^{G2′}—, —C(═O)—, —S(═O)—, —SO₂—, —C(═O)O—, —C(═O)NR^{G2′}—, —OC(═O)—, —NR^{G2′}C(O)—, —OC(O)O—, —OC(═O)NR^{G2′}—, —NR^{G2′}C(═O)O—, —NR^{G2′}C(═O)NR^{G3′}—, —C(═S)—, —C(═S)S—, —SC(═S)—, —SC(═S)S—, —C(═NR^{G2′})—, —C(═NR^{G2′})O—, —C(═NR^{G2′})R^{G3′}—, —OC(═NR^{G2′})—, —NR^{G2′}C(═NR^{G3′})—, —NR^{G2′}SO₂—, —C(═NR^{G2′})NR^{G3′}—, —OC(═NR^{G2′})—, —NR^{G2′}C(═NR^{G3′})—, —NR^{G2′}O₂—, —NR^{G2′}SO₂NR^{G3′}—, —NR^{G2′}C(═S)—, —SC(═S)NR^{G2′}—, —NR^{G2′}C(═S)S—, —NR^{G2′}C(═S)NR^{G3′}—, —SC(═NR^{G2′})—, —C(═S)NR^{G2′}—, —OC(═S)NR^{G2′}—, —NR^{G2′}C(═S)O—, —SC(═O)NR^{G2′}—, —NR^{G2′}C(O)S—, —C(O)S—, —SC(O)—, —SC(O)S—, —C(═S)O—, —OC(═S)—, —OC(═S)O—, —SO₂NR^{G2′}—, —BR^{G2′}—, or —PR^{G2′}—,

wherein each occurrence of R^{G1′}, R^{G2′}, and R^{G3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some forms, the ligands do not have the following structures:

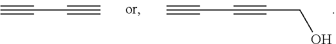

In some forms, the metal atom is Pt(II), and the ligand does not have the following structures:

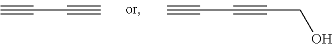

Each ligand can be independently in a native form or a deprotonated form.

3. Exemplary Formulas and Structures of the Compounds

In some forms, the metal complexes have a square-planar molecular geometry with monodentate, bidentate, tridentate or tetradentate ligands. In some forms, the compounds can have a structure of Formula I:

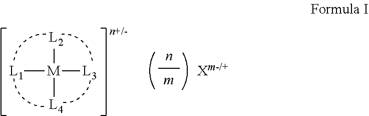

wherein

(a) M represents a metal atom selected from Pt(II), Pd(II), Ni(II), Ir(I), Rh(I), Au(III), Ag(III), and Cu(III),

(b) L₁, L₂, L₃, and L₄ represent ligands, wherein each ligand provides one donor atom to coordinate to the metal atom,

(c) n+/− represents the number of positive or negative charges carried by the metal complex in the formula, wherein n is zero or a positive integer, such as 1, 2, 3, 4, and 5,

(d) X^{m-/+} represents a counterion to maintain charge neutrality, wherein X^{m-/+} has a charge opposite to the charge of the metal complex and wherein m is zero or a positive integer, such as 1, 2, and 3, m=n or m≠n,

$$( e ) \frac{n}{m}$$

represents the stoichiometry of the counterion in the formula,

(f) dashed lines represent optional covalent linkages between the two ligands, optional fusion of ring moieties from the two ligands, or a combination thereof.

When X^{m-/+} is an anion, i.e., X^{m-}, it can be selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄⁻), sulfate (SO₄²⁻), hydrogenphosphate (HPO₄²⁻), phosphate (PO₄³⁻), and derivatives thereof. When X^{m-/+} is a cation, i.e., X^{m+}, it can be selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof.

Exemplary structures of Formula I include the following:

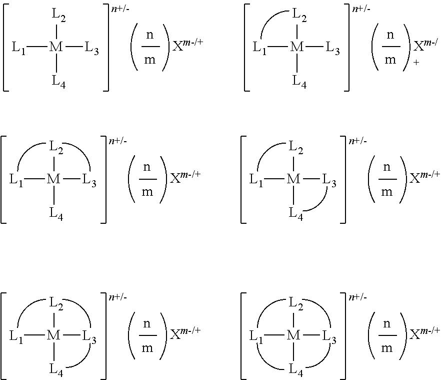

wherein the curved lines represent optional covalent linkages between the two ligands, optional fusion of ring moieties from the two ligands, or a combination thereof.

In some forms, the compound does not have the following structure.

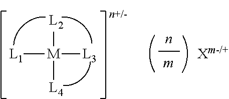

In some forms, L₁, L₂, and L₃ are independently selected from optionally substituted, and/or optionally deprotonated C₆-C₅₀ arenes or C₃-C₅₀ heteroarenes, such as 5-membered arene, 6-membered arene, 5-membered heteroarene, and 6-membered heteroarene. Examples of L₁, L₂, and L₃ include benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, and derivatives thereof.

In some forms, L₄ is selected from benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, halide, alkylamine, arylamine, alkylphosphine, arylphosphine, alkylarsine, arylarsine, C≡C—R⁻, SR⁻, OR⁻, SeR⁻, and derivatives thereof, wherein R is defined above. For example, R is selected from H or substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties). In some forms, L₄ is C≡C—R⁻.

In some forms, L₁ and L₂ are connected by covalent linkages, fusion of ring moieties from the two ligands, or a combination thereof. In some forms, L₂ and L₃ are further connected by covalent linkages, fusion of ring moieties from the two ligands, or a combination thereof. In some forms, L₁ and L₄ are further connected by covalent linkages, fusion of ring moieties from the two ligands, or a combination thereof. In some forms, L₃ and L₄ are further connected by covalent linkages, fusion of ring moieties from the two ligands, or a combination thereof.

In some forms, the metal atom is not Au(III). In some forms, the metal atom is Au(III), and the compound does not have the following structure:

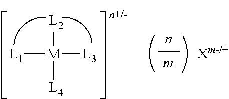

In some forms, the ligands do not have the following structures:

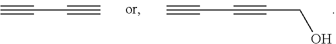

In some forms, the metal atom is Pt(II), and the ligand does not have the following structures:

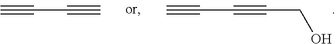

In some forms, the metal complexes have a linear planar molecular geometry. In some forms, the compounds can have a structure of Formula II:

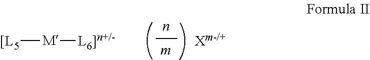

wherein M′ represents a metal atom selected from Ni(0), Pd(0), Pt(0), Cu(I), Ag(I), Au(I), Zn(II), Cd(II), and Hg(II),

wherein L₅ and L₆ represent ligands, wherein each ligand provides one donor atom to coordinate to the metal atom.

In some forms, the metal complexes have a trigonal-planar molecular geometry with monodentate, bidentate, tridentate ligands. In some forms, the compounds can also have a structure of Formula III:

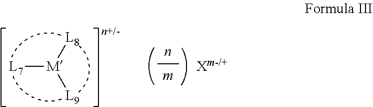

wherein L₇, L₈, and L₉ represent ligands, wherein each ligand provides one donor atom to coordinate to the metal atom.

Exemplary structures of Formula III include the following:

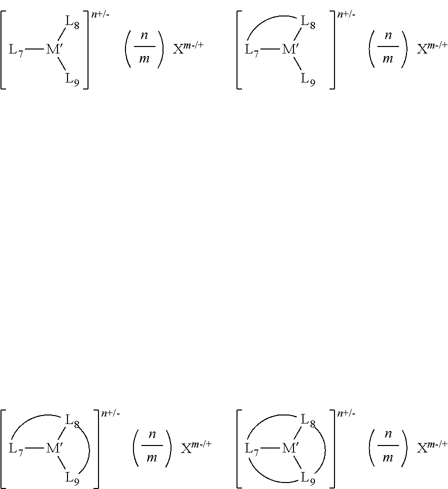

wherein the curved lines represent optional covalent linkages between the two ligands, optional fusion of ring moieties from the two ligands, or a combination thereof.

Exemplary structures of the metal complexes in the compounds of Formulas I, II, or, III are shown below.

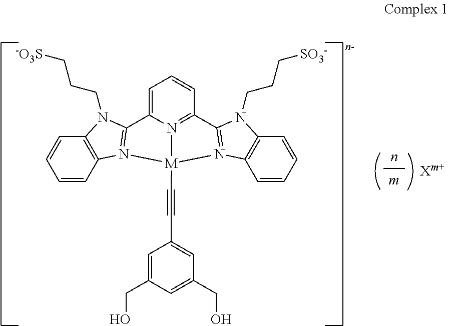

wherein M is Pt(II) (complex 1-Pt), Pd(II) (complex 1-Pd), Ni(II) (complex 1-Ni), Ir(I) (complex 1-Ir), Rh(I) (complex 1-Rh), Au(III) (complex 1-Au), Ag(III) (complex 1-Ag), or Cu(III) (complex 1-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

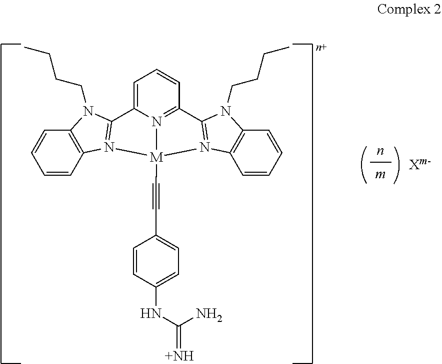

wherein M is Pt(II) (complex 2-Pt), Pd(II) (complex 2-Pd), Ni(II) (complex 2-Ni), Ir(I) (complex 2-Ir), Rh(I) (complex 2-Rh), Au(III) (complex 2-Au), Ag(III) (complex 2-Ag), or Cu(III) (complex 2-Cu),

wherein n+ is the number of positive charges carried by the metal complex in the formula, wherein n is a positive integer,

wherein X^{m-} is a counterion to maintain charge neutrality, wherein m is a positive integer, m=n or m≠n,

wherein

$$\frac{n}{m}$$

is the stoichiometry of the counterion in the formula.

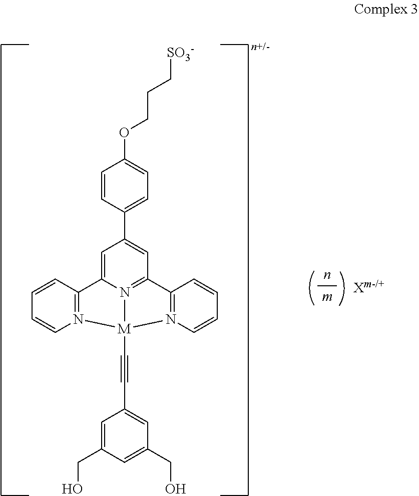

wherein M is Pt(II) (complex 3-Pt), Pd(II) (complex 3-Pd), Ni(II) (complex 3-Ni), Ir(I) (complex 3-Ir), Rh(I) (complex 3-Rh), Au(III) (complex 3-Au), Ag(III) (complex 3-Ag), or Cu(III) (complex 3-Cu),

wherein n+/− is the number of positive or negative charges carried by the metal complex in the formula, wherein n is zero or a positive integer,

wherein X^{m-/+} is a counterion to maintain charge neutrality, wherein m is zero or a positive integer, m=n or m≠n,

where

$$\frac{n}{m}$$

is the stoichiometry of the counterion in the formula.

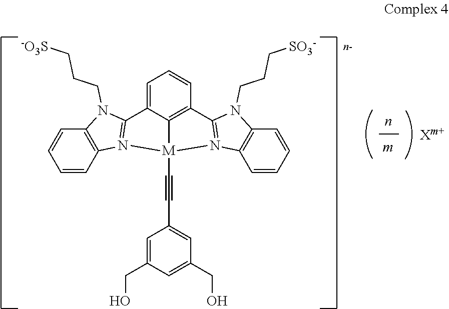

wherein M is Pt(II) (complex 4-Pt), Pd(II) (complex 4-Pd), Ni(II) (complex 4-Ni), Ir(I) (complex 4-Ir), Rh(I) (complex 4-Rh), Au(III) (complex 4-Au), Ag(III) (complex 4-Ag), or Cu(III) (complex 4-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

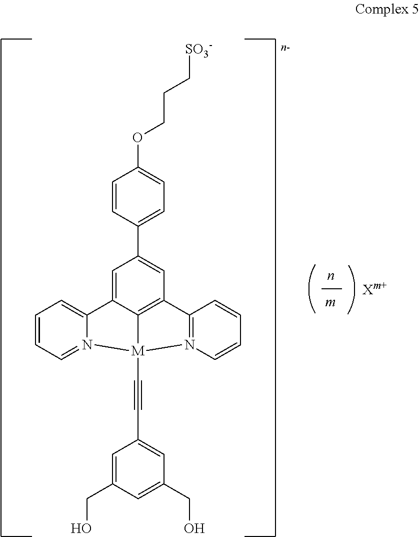

wherein M is Pt(II) (complex 5-Pt), Pd(II) (complex 5-Pd), Ni(II) (complex 5-Ni), Ir(I) (complex 5-Ir), Rh(I) (complex 5-Rh), Au(III) (complex 5-Au), Ag(III) (complex 5-Ag), or Cu(III) (complex 5-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

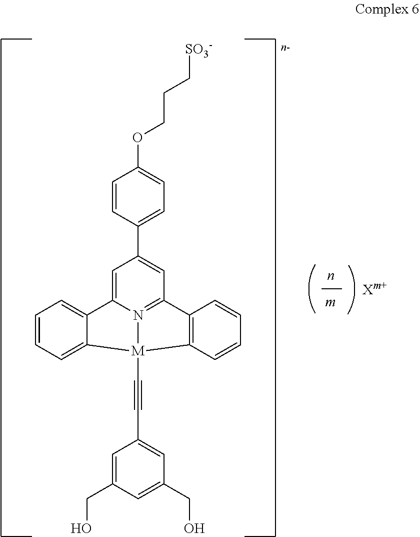

wherein M is Pt(II) (complex 6-Pt), Pd(II) (complex 6-Pd), Ni(II) (complex 6-Ni), Ir(I) (complex 6-Ir), Rh(I) (complex 6-Rh), Au(III) (complex 6-Au), Ag(III) (complex 6-Ag), or Cu(III) (complex 6-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

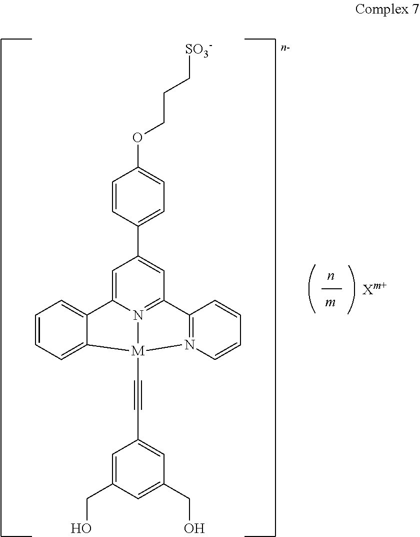

wherein M is Pt(II) (complex 7-Pt), Pd(II) (complex 7-Pd), Ni(II) (complex 7-Ni), Ir(I) (complex 7-Ir), Rh(I) (complex 7-Rh), Au(III) (complex 7-Au), Ag(III) (complex 7-Ag), or Cu(III) (complex 7-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

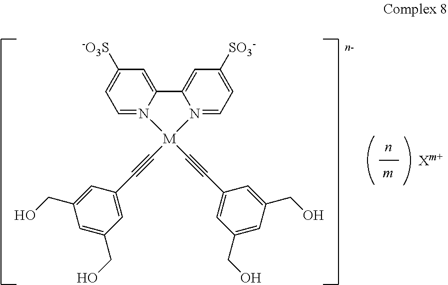

wherein M is Pt(II) (complex 8-Pt), Pd(II) (complex 8-Pd), Ni(II) (complex 8-Ni), Ir(I) (complex 8-Ir), Rh(I) (complex 8-Rh), Au(III) (complex 8-Au), Ag(III) (complex 8-Ag), or Cu(III) (complex 8-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

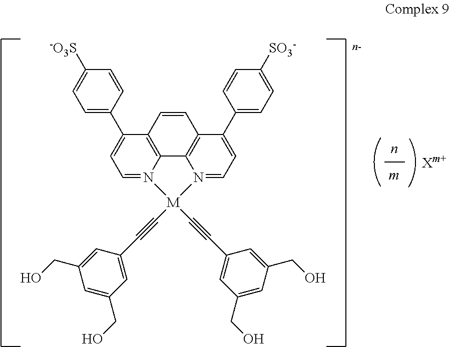

wherein M is Pt(II) (complex 9-Pt), Pd(II) (complex 9-Pd), Ni(II) (complex 9-Ni), Ir(I) (complex 9-Ir), Rh(I) (complex 9-Rh), Au(III) (complex 9-Au), Ag(III) (complex 9-Ag), or Cu(III) (complex 9-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

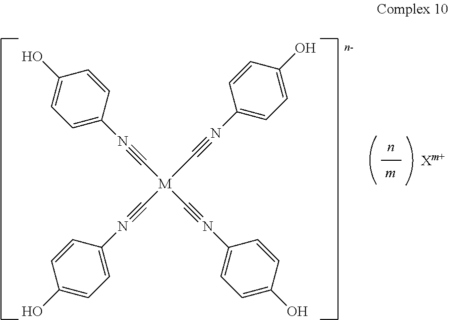

wherein M is Pt(II) (complex 10-Pt), Pd(II) (complex 10-Pd), Ni(II) (complex 10-Ni), Ir(I) (complex 10-Ir), Rh(I) (complex 10-Rh), Au(III) (complex 10-Au), Ag(III) (complex 10-Ag), or Cu(III) (complex 10-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

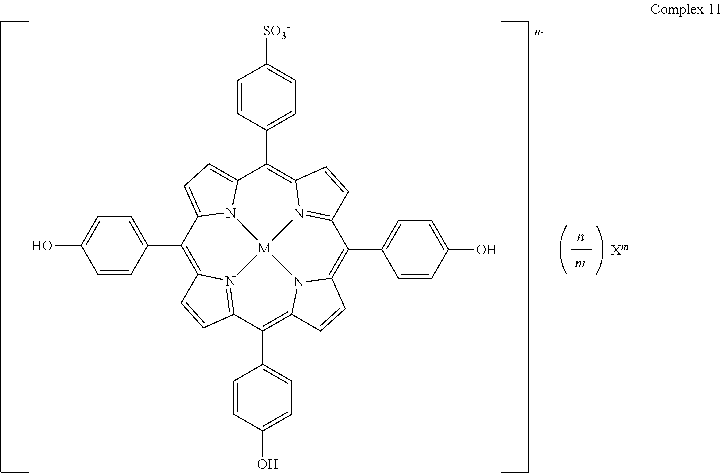

wherein M is Pt(II) (complex 11-Pt), Pd(II) (complex 11-Pd), Ni(II) (complex 11-Ni), Ir(I) (complex 11-Ir), Rh(I) (complex 11-Rh), Au(III) (complex 11-Au), Ag(III) (complex 11-Ag), or Cu(III) (complex 11-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

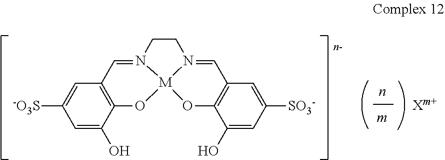

wherein M is Pt(II) (complex 12-Pt), Pd(II) (complex 12-Pd), Ni(II) (complex 12-Ni), Ir(I) (complex 12-Ir), Rh(I) (complex 12-Rh), Au(III) (complex 12-Au), Ag(III) (complex 12-Ag), or Cu(III) (complex 12-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

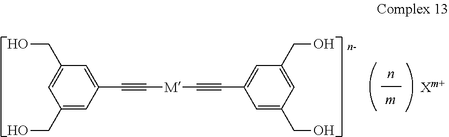

wherein M′ is Ni(0) (complex 13-Ni), Pd(0) (complex 13-Pd), Pt(0) (complex 13-Pt), Cu(I) (complex 13-Cu), Ag(I) (complex 13-Ag), Au(I) (complex 13-Au), Zn(II) (complex 13-Zn), Cd(II) (complex 13-Cd), or Hg(II) (complex 13-Hg),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

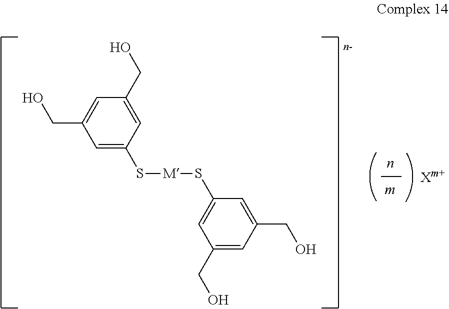

wherein M′ is Ni(0) (complex 14-Ni), Pd(0) (complex 14-Pd), Pt(0) (complex 14-Pt), Cu(I) (complex 14-Cu), Ag(I) (complex 14-Ag), Au(I) (complex 14-Au), Zn(II) (complex 14-Zn), Cd(II) (complex 14-Cd), or Hg(II) (complex 14-Hg),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

The compounds of Formulas I, II, or III can be readily synthesized using techniques generally known to synthetic organic and inorganic chemists. Exemplary methods to synthesize a specific compound of Formulas I, i.e., complex 1-Pt and complex 2-Pt, are described in the disclosed examples.

### III. MIXTURES AND COMPOSITIONS

Disclosed are mixtures, compositions, and kits formed by performing or preparing to perform the disclosed methods.

1. Mixtures and Compositions

For example, disclosed are mixtures containing a plurality of the compounds useful for detecting and/or imaging the analyte or screening and/or testing inhibitors. In some forms, the analyte is amyloid, plaque, or both, of proteins or peptides. In some forms, the analyte is RNA, nucleolus, or both. The mixtures can be used for screening or testing the efficacy of inhibitors against amyloidosis and/or fibrillar growth of proteins or peptides. The mixtures can be also used for nucleolus imaging.

In some forms, the mixtures contain a plurality of compounds with the structure of Formulas I, II, or III.

In some forms, the compounds in the mixtures can have different specificities towards different types of amyloid or plaque. Alternatively, the compounds in the mixtures can have different specificities towards amyloid, plaque, or both, of different proteins or peptides. The compounds in the mixtures can exhibit different photophysical properties after aggregation and supramolecular self-assembly, thereby enabling simultaneous detection and/or imaging of different types of amyloid or plaque and/or simultaneous detection and/or imaging of amyloid, plaque, or both, of different proteins or peptides.

In some forms, the compounds in the mixtures can also have different specificities towards different types of RNA. The compounds in the mixtures can exhibit different photophysical properties after aggregation and supramolecular self-assembly, thereby enabling simultaneous detection of different types of RNA.

In another example, disclosed are compositions containing one or more of the disclosed compounds, such as compounds with the structure of Formulas I, II, or III, together with one or more other compounds, solvents, or materials. In some forms, the one or more other compounds, solvents, or materials can improve the performance and/or increase the stability of the disclosed compounds. The compositions can be in the form of solutions, suspensions, emulsions, powders, or solid cakes.

2. Kits

The compounds, mixtures, and compositions described above can be packaged together with other components in any suitable combination as a kit useful for performing, or aiding in the performance of, the disclosed methods. It is useful if the components in a given kit are designed and adapted for use together in the disclosed methods.

The kits contain, in one or more containers, one or more of the disclosed compounds, mixtures, and compositions. The kits can also contain one or more other components, such as compounds, solvents, and materials, as carriers. The carriers do not interfere with the effectiveness of the disclosed compounds in performing their functions. The kits can include instructions for use.

The kits can be used to detect and/or image the analyte. In some forms, the analyte is amyloid, plaque, or both, of proteins or peptides. In some forms, the analyte is RNA, nucleolus, or both. The kits can be used for screening or testing the efficacy of inhibitors against amyloidosis and/or fibrillar growth of proteins or peptides. The kits can be also used for nucleolus imaging.

The kits can also include one or more positive controls. In some forms, the positive control is a solution, suspension, or dry powder of amyloid, plaque, or both, of one or more proteins or peptides. In some forms, the positive control is a solution, suspension, or dry powder of RNA.

### IV. METHODS OF USE

Disclosed are methods for detecting and/or imaging an analyte or screening and/or testing inhibitors.

In some forms, the analyte is amyloid, plaque, or both, of proteins or peptides. In some forms, the analyte is RNA, nucleolus, or both. The compounds can be used for screening or testing the efficacy of inhibitors against amyloidosis and/or fibrillar growth of proteins or peptides. The compounds can be also used for nucleolus imaging.

For example, method for detecting an analyte in a sample can include (a) combining one or more of the disclosed compounds with the sample and (b) detecting changes in the photophysical properties of the metal complexes of the compounds. Detection of changes in the photophysical properties of the metal complexes indicates the presence of aggregation and supramolecular self-assembly of the metal complexes, wherein the presence of aggregation and supramolecular self-assembly of the metal complexes indicates the presence of the analyte in the sample.

For example, methods for imaging an analyte in a sample can include (a) combining one or more of the disclosed compounds with the sample under conditions to allow for binding of the metal complexes of the compounds with the analyte and subsequent aggregation and supramolecular self-assembly of the metal complex, wherein aggregation and supramolecular self-assembly of the metal complexes generates changes in the photophysical properties of the metal complexes, and (b) imaging the analyte based on one or more photophysical properties that are specific for the metal complexes after aggregation and supramolecular self-assembly.

1. Detecting and/or Imaging Amyloid, Plaque, or Both, of Proteins or Peptides

Disclosed are methods to detect and/or image amyloid, plaque, or both, of one or more proteins or peptides in a sample containing the protein or peptide. The methods include (a) combining one or more of the disclosed compounds with the sample and (b) detecting changes in the photophysical properties of the metal complexes of the compounds. Detection of changes in the photophysical properties of the metal complexes indicates the presence of aggregation and supramolecular self-assembly of the metal complexes, wherein the presence of aggregation and supramolecular self-assembly of the metal complexes indicates the presence of amyloid, plaque, or both, of the proteins or peptides in the sample.

Depending on the types of changes in the photophysical properties of the metal complexes, detection and/or imaging of the amyloid, plaque, or both, of the protein or peptide can be conducted using different techniques, such as colorimetric assay, luminescence assay, RLS analysis or combinations thereof.

In some forms, detection and/or imaging of the amyloid, plaque, or both, of the protein or peptide can be conducted using a luminescence turn-on assay as illustrated in FIG. 13. Aggregation and supramolecular self-assembly of the metal complex, induced by binding of the metal complex to the amyloid, plaque, or both, of the protein or peptide, can induce an increase in its luminescence intensity. The increase in the luminescence intensity can be caused by aggregation and supramolecular self-assembly of the metal complexes via noncovalent metal-metal interactions, similar to the effect of excitonic coupling. Noncovalent interactions such as π-π stacking interactions, electrostatic interactions, hydrogen bonding interactions, hydrophobic interactions, and combinations thereof can contribute to the binding of the metal complexes to the amyloid, plaque, or both, of the protein or peptide, which leads to the aggregation and supramolecular self-assembly of the metal complexes.

In some forms, the sample is measured using a non-imaging spectrometer such as from a cuvette, small sample holder or a multi-well plate. In some forms, the sample is measured using an imaging spectrometer such as confocal microscopy.

In some forms, the methods also include performing parallel measurements on one or more samples of amyloid, plaque, or both, of other proteins or peptides or on one or more samples of amyloid, plaques, or both, of the same protein or peptide, wherein the structural properties of the amyloid, plaque, or both, in these samples, are previously known and/or characterized. By conducting such measurements using an amyloid or plaque of known structural properties, the combined set of information can provide a way to deduce the structural properties of the amyloid, plaque, or both, in the sample that is under investigation.

Also disclosed are methods to investigate the process of amyloid fibrillation and plaque formation of one or more proteins or peptides under different conditions. The methods include (a) combining one or more of the disclosed compounds with samples of the protein or peptide collected or prepared at different times and/or under different conditions and (b) comparing the photophysical properties of the metal complexes among the samples. The differences in the photophysical properties of the metal complexes among the samples represent the differences in the extents or stages of amyloid fibrillation and plaque formation. The kinetics for amyloid fibrillation and plaque formation can be deduced from time-dependent comparisons of the photophysical properties of the metal complexes among the samples collected or prepared at different times.

2. Evaluating the Efficacy of Inhibitors Against Amyloidosis and/or Fibrillar Growth of Proteins or Peptides

Disclosed are methods to test the efficacy of inhibitors against amyloidosis and/or fibrillar growth of one or more proteins or peptides. The methods include (a) combining one or more of the disclosed compounds with an inhibitor-treated sample containing the proteins or peptides and, separately, with an untreated sample containing the proteins or peptides and (b) comparing the photophysical properties of the metal complexes of the compounds between the two samples. The magnitude of the difference in the photophysical properties of the metal complexes between the two samples indicates the extent of change in the state of aggregation and supramolecular self-assembly of the metal complexes; the extent of change in the state of aggregation and supramolecular self-assembly of the metal complexes indicates the efficacy of the inhibitors.

In some forms, the inhibitor-treated sample can be prepared by treating the sample containing the protein and peptide with one or more inhibitors for a time period sufficient for the inhibitor to function. The inhibitor can be added before, during, or after amyloidosis and/or fibrillar growth of the protein or peptide.

Also disclosed are methods for screening inhibitors against amyloidosis and/or fibrillar growth of one or more proteins or peptides. The methods involve evaluating and then comparing the efficacies of the inhibitors against amyloidosis and/or fibrillar growth of the protein or peptide.

3. Detecting RNA and Imaging Nucleolus

Disclosed are methods to detect RNA, nucleolus, or both in a sample. The methods include (a) combining one or more of the disclosed compounds with the sample and (b) detecting changes in the photophysical properties of the metal complexes of the compounds. Detection of changes in the photophysical properties of the metal complexes indicates the presence of aggregation and supramolecular self-assembly of the metal complexes, wherein the presence of aggregation and supramolecular self-assembly of the metal complexes indicates the presence of RNA, nucleolus, or both, in the sample.

Depending on the types of changes in the photophysical properties of the metal complexes, detection of RNA and nucleolus imaging can be conducted using different techniques, such as colorimetric assay, luminescence assay, RLS analysis or combinations thereof.

In some forms, detection of RNA and nucleolus imaging can be conducted using a luminescence turn-on assay as illustrated in FIG. 30. Aggregation and supramolecular self-assembly of the metal complex, induced by binding of the metal complex to RNA, nucleolus, or both, can induce an increase in its luminescence intensity. The increase in the luminescence intensity can be caused by aggregation and supramolecular self-assembly of the metal complexes via noncovalent metal-metal interactions, similar to the effect of excitonic coupling. Noncovalent interactions such as π-π stacking interactions, electrostatic interactions, hydrogen bonding interactions, hydrophobic interactions, and combinations thereof can contribute to the binding of the metal complexes to RNA, nucleolus, or both, which leads to the aggregation and supramolecular self-assembly of the metal complexes.

In some forms, the sample is measured using a non-imaging spectrometer such as from a cuvette, small sample holder or a multi-well plate. In some forms, the sample is measured using an imaging spectrometer such as confocal microscopy.

Methods of imaging nucleolus in a sample are disclosed. The methods include (a) combining one or more of the disclosed compounds with the sample under conditions to allow for binding of the metal complexes of the compounds with the nucleolus and subsequent aggregation and supramolecular self-assembly of the metal complexes, wherein aggregation and supramolecular self-assembly of the metal complexes generates changes in the photophysical properties of the metal complexes of the compounds, and (b) imaging the nucleolus based on one or more photophysical properties that are specific for the metal complexes after aggregation and supramolecular self-assembly.

In some forms, the sample contains eukaryotic cells. The cell can be, but not limited to, 3T3 cells, A549 cells, Chinese hamster ovary (CHO) cells, HEK 293 cells, HeLa cells, Hep G2 cells, and HT1080 cells.

In some forms, the sample is imaged using an imaging spectrometer such as confocal microscopy.

4. Combinational Use

The disclosed methods also include combinational use of more than one of the disclosed compounds. The compounds can be combined to form mixtures or compositions as described previously.

In some forms, the compounds in the mixtures can have different specificities towards different types of amyloid or plaque. Alternatively, the compounds in the mixtures can have different specificities towards amyloid, plaque, or both, of different proteins or peptides. The compounds in the mixtures can exhibit different photophysical properties after aggregation and supramolecular self-assembly, thereby enabling simultaneous detection and/or imaging of different types of amyloid or plaque and/or simultaneous detection and/or imaging of amyloid, plaque, or both, of different proteins or peptides.

In some forms, the compounds in the mixtures can also have different specificities towards different types of RNA. The compounds in the mixtures can exhibit different photophysical properties after aggregation and supramolecular self-assembly, thereby enabling simultaneous detection of different types of RNA.

5. Samples

In some forms, the sample contains one or more proteins or peptides. The protein or peptide can be isolated protein or peptide. In some forms, the sample containing the protein or peptide can be or contain a human or non-human animal bodily fluid, a human or non-human animal tissue, or a combination thereof. Exemplary bodily fluids include saliva, sputum, blood serum, blood, urine, mucus, vaginal lubrication, pus, cerebrospinal fluid, and wound exudate. In some forms, the bodily fluid is cerebrospinal fluid. Exemplary tissues include organ tissues and non-organ tissues, such as brain tissue, heart tissue, kidney tissue, liver tissue, eye tissue, tongue tissue, and pancreas tissue. In some forms, the tissue is brain tissue. The human or non-human animal tissue can be lysed to prepare for the sample.

The amyloid, plaque, or both, of the protein or peptide in the sample can contain thread-like aggregates of the protein or peptide, which are ordered in a β-sheet conformation.

The amyloid, plaque, or both, of the protein or peptide in the sample can be analyzed directly, or can be amplified prior to analysis. In some forms, one or more additional steps can be performed to induce the formation of amyloid, plaque, or both, of the protein or peptide in the sample. In some forms, the formation of amyloid, plaque, or both, of the protein or peptide can involve denaturation of the protein or peptide. In some forms, the formation of amyloid, plaque, or both, of the protein or peptide can be induced by chemical approaches, physical approaches, or both. Exemplary chemical approaches include adding one or more chemical compound, such as transition metal ions, to the sample, adding a specific solvent, such as ethanol and methanol, to the sample, preparing the sample by dissolving the proteins or peptides in a specific solvent, altering the pH of the sample, and preparing the sample by dissolving the proteins or peptides in a specific pH range, such as acidic or basic conditions. Exemplary physical approaches include changing the temperature of the sample, such as increasing the temperature, and introducing physical disturbance to the sample, such as stirring, shaking, or vortexing the sample.

In some forms, the sample contains one or more proteins or peptides selected from, but not limited to, amyloid-β peptide, α-synuclein, insulin, huntingtin, tau protein, hyperphosphorylated tau protein (pTau), prion protein, IAPP (amylin), calcitonin, PrP^{Sc} atrial natriuretic factor, apolipoprotein A1, serum amyloid A, medin, prolactin, transthyretin, lysozyme, beta-2 microglobulin, gelsolin, karatoepithelin, crystallin, desmin, selenoproteins, actin, cystatin, immunoglobulin light chain AL, S-IBM, and myosin. In some forms, the sample contains amyloid, plaque, or both, of one or more of these proteins or peptides. In some forms, the sample contains amyloid, plaque, or both, of amyloid-β peptide.

In some forms, the sample is obtained from a patient. In some forms, the patient has one or more diseases or disorders associated with amyloidosis and proteopathy. In some forms, the disease or disorder is selected from, but not limited to, Alzheimer's disease, Parkinson's disease, injection-localized amyloidosis, Huntington's disease, mild cognitive impairment, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, and Down syndrome. In some forms, the disease or disorder is Alzheimer's disease.

In some forms, the sample contains RNA, nucleolus, or both. In some forms, the RNA is isolated RNA from biological sources. In some forms, the sample contains or is derived from eukaryotic cells. Exemplary eukaryotic cells include, but are not limited to, 3T3 cells, A549 cells, Chinese hamster ovary (CHO) cells, HEK 293 cells, HeLa cells, Hep G2 cells, and HT1080 cells.

The RNA in the sample can be analyzed directly, or can be amplified prior to analysis.

In some forms, the sample contains one or more types of RNA selected from, but not limited to, messenger RNA (mRNA), transfer RNA (tRNA), ribosomal RNA (rRNA), transfer-messenger RNA (tmRNA), antisense RNA (asRNA), enhancer RNA (eRNA), guide RNA (gRNA), ribozyme, short hairpin RNA (shRNA), small temporal RNA (stRNA), small interfering RNA (siRNA), and trans-acting siRNA (ta-siRNA).

6. Diagnosis of Amyloidosis and Proteopathy

Disclosed are methods for diagnosis of one or more diseases or disorders associated with amyloidosis and proteopathy in a patient in need thereof.

In some forms, the disease or disorder is selected from, but not limited to, Alzheimer's disease, Parkinson's disease, injection-localized amyloidosis, Huntington's disease, mild cognitive impairment, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, and Down syndrome. In some forms, the disease or disorder is Alzheimer's disease.

In some forms, the methods include (a) extracting a sample from the patient; and (b) detecting and/or imaging the presence of amyloid, plaque, or both of one or more proteins or peptides using one or more of the disclosed compounds. In some forms, step (b) further includes quantifying the amount of amyloid, plaque, or both of the protein or peptide. In some forms, the methods include detecting and/or imaging the presence of amyloid, plaque, or both of one or more proteins or peptides using one or more of the disclosed compounds. In some forms, the method further includes quantifying the amount of amyloid, plaque, or both of the protein or peptide.

In some forms, the sample from the patient contains a bodily fluid, a tissue, or a combination thereof. The bodily fluid can be cerebrospinal fluid; the tissue can be brain tissue.

In some forms, the sample from the patient contains one or more proteins or peptides selected from, but not limited to, amyloid-O peptide, α-synuclein, insulin, huntingtin, tau protein, hyperphosphorylated tau protein (pTau), prion protein, IAPP (amylin), calcitonin, PrP^{Sc}, atrial natriuretic factor, apolipoprotein A1, serum amyloid A, medin, prolactin, transthyretin, lysozyme, beta-2 microglobulin, gelsolin, karatoepithelin, crystallin, desmin, selenoproteins, actin, cystatin, immunoglobulin light chain AL, S-IBM, and myosin. In some forms, the sample contains amyloid, plaque, or both, of one or more of these proteins or peptides. In some forms, the sample contains amyloid, plaque, or both, of amyloid-β peptide.

In some forms, the methods include in vivo imaging. In some forms, the in vivo imaging involves (a) administering one or more of the disclosed compounds to the patient, systematically or to a specific bodily region; (b) detecting the presence of amyloid, plaque, or both of one or more proteins or peptides using fluorescence imaging, systematically or in the specific bodily region. In some forms, the specific bodily region is in the brain.

The disclosed compositions and methods can be further understood through the following numbered paragraphs.

1. A compound for detecting and/or imaging an analyte, wherein the compound is a d⁸ or d¹⁰ metal complex or a salt thereof, comprising:
(a) a metal atom with a coordination number of 2, 3, or 4, selected from the group consisting of Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), and Hg(II); and
(b) one or more ligands with donor atoms independently selected from the group consisting of carbon (C), nitrogen (N), oxygen (O), phosphorus (P), sulfur (S), arsenic (As), and selenium (Se),

wherein the metal complex binds to the analyte, wherein binding of the metal complex to the analyte induces aggregation and supramolecular self-assembly of the metal complex through noncovalent metal-metal interactions.

2. The compound of paragraph 1, wherein the compound has a structure of Formula I:

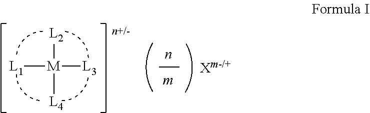

wherein
(a) M represents a metal atom selected from Pt(II), Pd(II), Ni(II), Ir(I), Rh(I), Au(III), Ag(III), and Cu(III),
(b) L₁, L₂, L₃, and L₄ represent ligands, wherein each ligand provides one donor atom to coordinate to the metal atom,
(c) n+/− represents the number of positive or negative charges carried by the metal complex in the formula, wherein n is zero or a positive integer,
(d) X^{m-/+} represents a counterion to maintain charge neutrality, wherein X^{m-/+} has a charge opposite to the charge of the metal complex and wherein m is zero or a positive integer, m=n or m≠n,

$$( e ) \frac{n}{m}$$

represents the stoichiometry of the counterion in the formula,
(f) dashed lines represent optional covalent linkages between the two ligands, optional fusion of ring moieties from the two ligands, or a combination thereof.

3. The compound of paragraph 2, wherein L₁, L₂, and L₃ are optionally substituted, and/or optionally deprotonated C₆-C₅₀ arenes or C₃-C₅₀ heteroarenes, comprising benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, and derivatives thereof.

4. The compound of paragraph 2 or paragraph 3, wherein L₁ and L₂ are connected by covalent linkages, fusion of ring moieties from the two ligands, or a combination thereof.

5. The compound of paragraph 1, wherein the compound has a structure of Formula II:

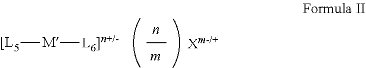

wherein M′ represents a metal atom selected from Ni(0), Pd(0), Pt(0), Cu(I), Ag(I), Au(I), Zn(II), Cd(II), and Hg(II),

wherein L₅ and L₆ represent ligands, wherein each ligand provides one donor atom to coordinate to the metal atom.

6. The compound of paragraph 1, wherein the compound has a structure of Formula III:

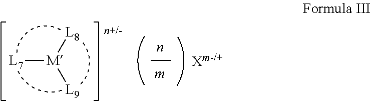

wherein L₇, L₈, and L₉ represent ligands, wherein each ligand provides one donor atom to coordinate to the metal atom.

7. The compound of any one of paragraphs 1-6, wherein the metal complex binds to the analyte via noncovalent interactions, wherein the noncovalent interactions comprise electrostatic interactions, hydrogen bonding interactions, hydrophobic interactions, or combinations thereof.

8. The compound of any one of paragraphs 1-7, wherein the metal complex has a planar structure or a partially planar structure.

9. The compound of any one of paragraphs 1-8, wherein the aggregation and supramolecular self-assembly of the metal complex creates one or more changes in the photophysical properties of the metal complex.

10. The compound of paragraph 9, wherein the changes in the photophysical properties comprise a change in optical absorbance, luminescence, resonance light scattering (RLS), or combinations thereof.

11. The compound of paragraph 10, wherein the change in luminescence comprises an increase in the luminescence quantum yield and/or emission intensity, and/or a shift in emission energy or wavelength.

12. The compound of any one of paragraphs 1-11, wherein the compound is selected from:

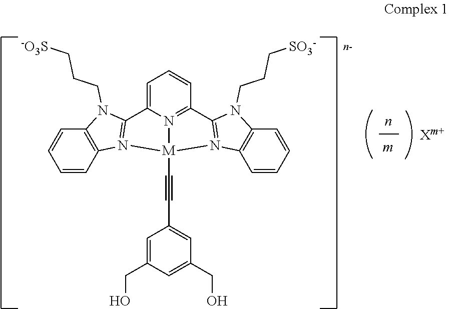

wherein M is Pt(II) (complex 1-Pt), Pd(II) (complex 1-Pd), Ni(II) (complex 1-Ni), Ir(I) (complex 1-Ir), Rh(I) (complex 1-Rh), Au(III) (complex 1-Au), Ag(III) (complex 1-Ag), or Cu(III) (complex 1-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

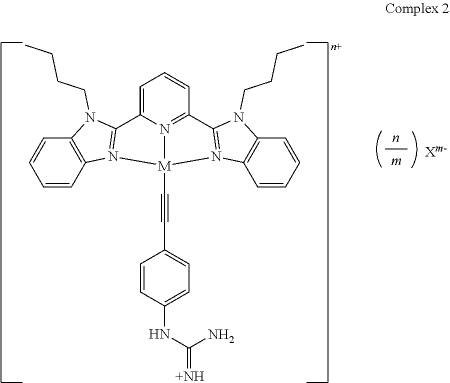

wherein M is Pt(II) (complex 2-Pt), Pd(II) (complex 2-Pd), Ni(II) (complex 2-Ni), Ir(I) (complex 2-Ir), Rh(I) (complex 2-Rh), Au(III) (complex 2-Au), Ag(III) (complex 2-Ag), or Cu(III) (complex 2-Cu),

wherein n+ is the number of positive charges carried by the metal complex in the formula, wherein n is a positive integer,

wherein X^{m-} is a counterion to maintain charge neutrality, wherein m is a positive integer, m=n or m≠n,

wherein

$$\frac{n}{m}$$

is the stoichiometry of the counterion in the formula.

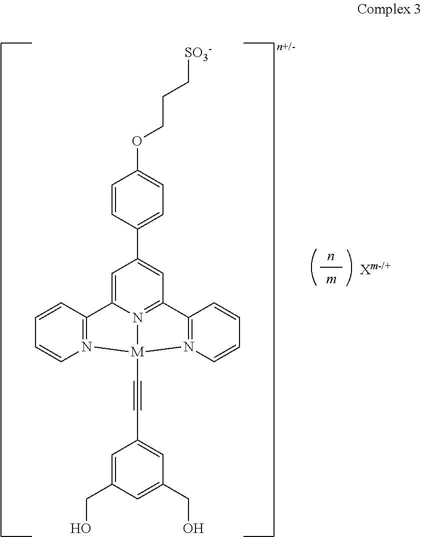

wherein M is Pt(II) (complex 3-Pt), Pd(II) (complex 3-Pd), Ni(II) (complex 3-Ni), Ir(I) (complex 3-Ir), Rh(I) (complex 3-Rh), Au(III) (complex 3-Au), Ag(III) (complex 3-Ag), or Cu(III) (complex 3-Cu),

wherein n+/− is the number of positive or negative charges carried by the metal complex in the formula, wherein n is zero or a positive integer,

wherein X^{m-/+} is a counterion to maintain charge neutrality, wherein m is zero or a positive integer, m=n or m≠n,

wherein

$$\frac{n}{m}$$

is the stoichiometry of the counterion in the formula.

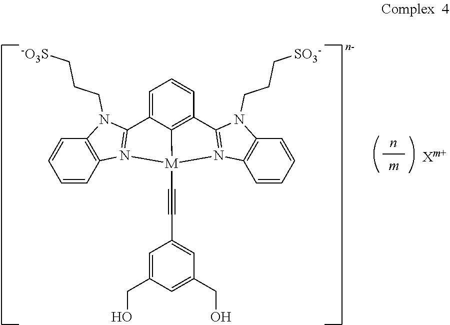

wherein M is Pt(II) (complex 4-Pt), Pd(II) (complex 4-Pd), Ni(II) (complex 4-Ni), Ir(I) (complex 4-Ir), Rh(I) (complex 4-Rh), Au(III) (complex 4-Au), Ag(III) (complex 4-Ag), or Cu(III) (complex 4-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

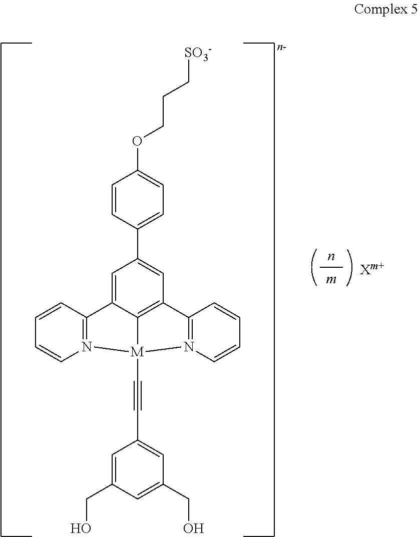

wherein M is Pt(II) (complex 5-Pt), Pd(II) (complex 5-Pd), Ni(II) (complex 5-Ni), Ir(I) (complex 5-Ir), Rh(I) (complex 5-Rh), Au(III) (complex 5-Au), Ag(III) (complex 5-Ag), or Cu(III) (complex 5-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

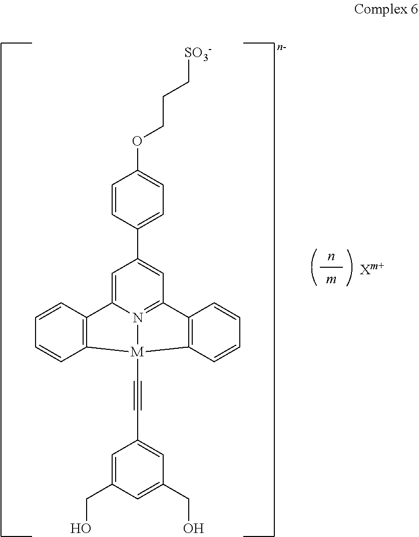

wherein M is Pt(II) (complex 6-Pt), Pd(II) (complex 6-Pd), Ni(II) (complex 6-Ni), Ir(I) (complex 6-Ir), Rh(I) (complex 6-Rh), Au(III) (complex 6-Au), Ag(III) (complex 6-Ag), or Cu(III) (complex 6-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

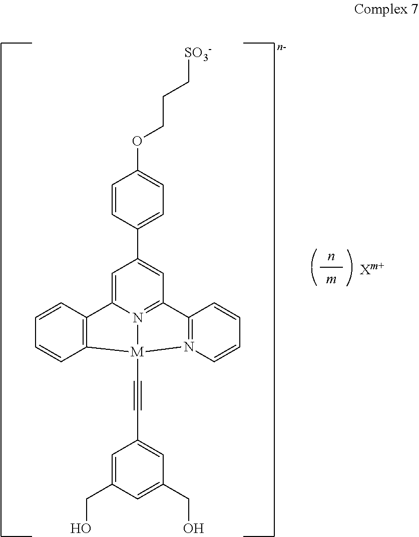

wherein M is Pt(II) (complex 7-Pt), Pd(II) (complex 7-Pd), Ni(II) (complex 7-Ni), Ir(I) (complex 7-Ir), Rh(I) (complex 7-Rh), Au(III) (complex 7-Au), Ag(III) (complex 7-Ag), or Cu(III) (complex 7-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

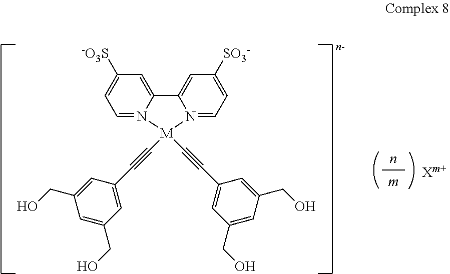

wherein M is Pt(II) (complex 8-Pt), Pd(II) (complex 8-Pd), Ni(II) (complex 8-Ni), Ir(I) (complex 8-Ir), Rh(I) (complex 8-Rh), Au(III) (complex 8-Au), Ag(III) (complex 8-Ag), or Cu(III) (complex 8-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

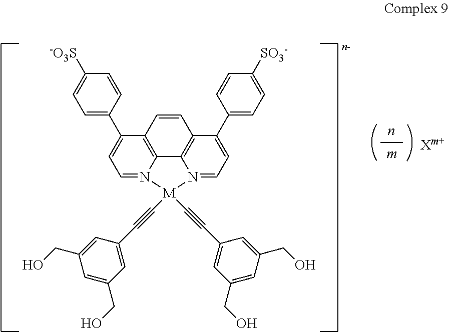

wherein M is Pt(II) (complex 9-Pt), Pd(II) (complex 9-Pd), Ni(II) (complex 9-Ni), Ir(I) (complex 9-Ir), Rh(I) (complex 9-Rh), Au(III) (complex 9-Au), Ag(III) (complex 9-Ag), or Cu(III) (complex 9-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

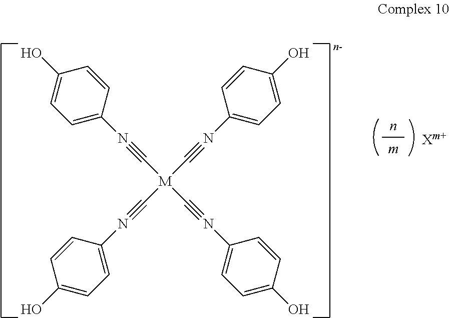

wherein M is Pt(II) (complex 10-Pt), Pd(II) (complex 10-Pd), Ni(II) (complex 10-Ni), Ir(I) (complex 10-Ir), Rh(I) (complex 10-Rh), Au(III) (complex 10-Au), Ag(III) (complex 10-Ag), or Cu(III) (complex 10-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

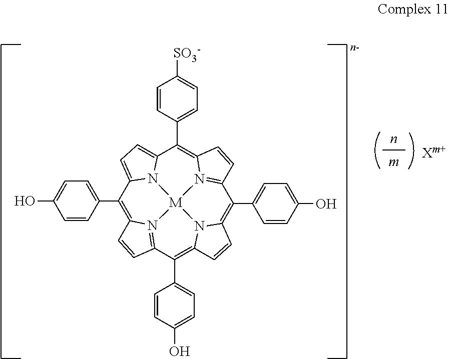

wherein M is Pt(II) (complex 11-Pt), Pd(II) (complex 11-Pd), Ni(II) (complex 11-Ni), Ir(I) (complex 11-Ir), Rh(I) (complex 11-Rh), Au(III) (complex 11-Au), Ag(III) (complex 11-Ag), or Cu(III) (complex 11-Cu),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

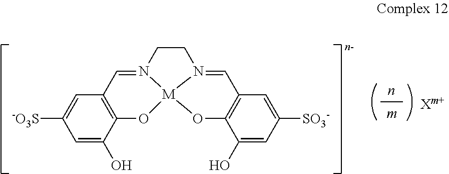

wherein M is Pt(II) (complex 12-Pt), Pd(II) (complex 12-Pd), Ni(II) (complex 12-Ni), Ir(I) (complex 12-Ir), Rh(I) (complex 12-Rh), Au(III) (complex 12-Au), Ag(III) (complex 12-Ag), or Cu(III) (complex 12-Cu),

wherein n− is the number of negative charges carried by the metal complex in the formula, wherein n is a positive integer,

wherein X^{m+} is a counterion to maintain charge neutrality, wherein m is a positive integer, m=n or m≠n,

wherein

$$\frac{n}{m}$$

is the stoichiometry of the counterion in the formula.

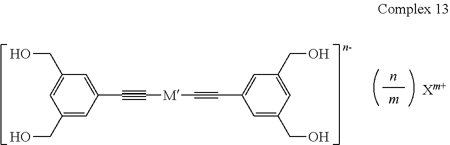

wherein M′ is Ni(0) (complex 13-Ni), Pd(0) (complex 13-Pd), Pt(0) (complex 13-Pt), Cu(I) (complex 13-Cu), Ag(I) (complex 13-Ag), Au(I) (complex 13-Au), Zn(II) (complex 13-Zn), Cd(II) (complex 13-Cd), or Hg(II) (complex 13-Hg),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

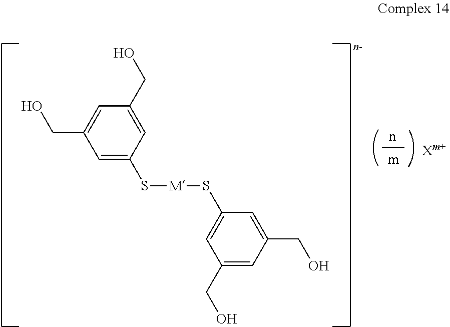

wherein M′ is Ni(0) (complex 14-Ni), Pd(0) (complex 14-Pd), Pt(0) (complex 14-Pt), Cu(I) (complex 14-Cu), Ag(I) (complex 14-Ag), Au(I) (complex 14-Au), Zn(II) (complex 14-Zn), Cd(II) (complex 14-Cd), or Hg(II) (complex 14-Hg),

wherein n− is the number of negative charges carried by the metal complex in the formula, wherein n is zero or a positive integer,

wherein X^{m+} is a counterion to maintain charge neutrality, wherein m is zero or a positive integer, m=n or m≠n,

wherein

$$\frac{n}{m}$$

is the stoichiometry of the counterion in the formula.

13. The compound of any one of paragraphs 1-12, wherein the analyte is selected from (1) amyloid, plaque, or both, of a protein or peptide and (2) RNA, nucleolus or both.

14. A method for detecting an analyte in a sample, comprising:

(a) combining the compound of any one of paragraphs 1-13 with the sample,

(b) detecting changes in the photophysical properties of the metal complex,

wherein detection of changes in the photophysical properties of the metal complex indicates the presence of aggregation and supramolecular self-assembly of the metal complex, wherein the presence of aggregation and supramolecular self-assembly of the metal complex indicates the presence of the analyte in the sample.

15. The method of paragraph 14, wherein the analyte is selected from (1) amyloid, plaque, or both, of a protein or peptide and (2) RNA, nucleolus or both.

16. The method of paragraph 14 or paragraph 15, wherein the sample comprises a human or non-human animal bodily fluid, a human or non-human animal tissue, or a combination thereof.

17. The method of paragraph 16, wherein the bodily fluid is cerebrospinal fluid.

18. The method of paragraph 16, wherein the tissue is brain tissue.

19. The method of any one of paragraphs 14-18, wherein analyte is the amyloid, plaque, or both, of a protein or peptide, wherein the amyloid, plaque, or both, of the protein or peptide in the sample comprises thread-like aggregates of the protein or peptide, which are ordered in a β-sheet conformation.

20. A method for testing the efficacy of an inhibitor against amyloidosis and/or fibrillar growth of a protein or peptide, comprising:

(a) combining the compound of any one of paragraphs 1-13 with an inhibitor-treated sample containing the protein or peptide and, separately, with an untreated sample containing the protein or peptide,

(b) comparing the photophysical properties of the metal complex between the two samples,

wherein the magnitude of the difference in the photophysical properties of the metal complex between the two samples indicates the extent of change in the state of aggregation and supramolecular self-assembly of the metal complex, wherein the extent of change in the state of aggregation and supramolecular self-assembly of the metal complex indicates the efficacy of the inhibitor.

21. A method for imaging an analyte in a sample, comprising:

(a) combining the compound of any one of paragraphs 1-13 with the sample under conditions to allow for binding of the metal complex of the compound with the analyte and subsequent aggregation and supramolecular self-assembly of the metal complex, wherein aggregation and supramolecular self-assembly of the metal complex generates changes in the photophysical properties of the metal complex,

(b) imaging the analyte based on one or more photophysical properties that are specific for the metal complex after aggregation and supramolecular self-assembly.

22. The method of paragraph 21, wherein the analyte is selected from (1) amyloid, plaque, or both, of a protein or peptide and (2) RNA, nucleolus or both.

23. The method of paragraph 21 or paragraph 22, wherein the sample contains eukaryotic cells optionally selected from the groups consisting of 3T3 cells, A549 cells, Chinese hamster ovary (CHO) cells, HEK 293 cells, HeLa cells, Hep G2 cells, and HT1080 cells.

24. A kit for use in detecting and/or imaging an analyte, comprising, in one or more containers, one or more compounds of any one of paragraphs 1-13 and optionally instructions for use.

25. The kit of paragraph 24, wherein the analyte is selected from (1) amyloid, plaque, or both, of a protein or peptide and (2) RNA, nucleolus or both.

26. The kit of paragraph 24 or paragraph 25, further comprising a carrier.

27. The kit of any one of paragraphs 24-26, wherein the presence of the analyte can induce aggregation and supramolecular self-assembly of the metal complex thereon after binding, wherein the aggregation and supramolecular self-assembly of the metal complex can be detected by changes in the photophysical properties of the metal complex.

### V. EXAMPLES

### Example 1. Synthesis and Characterization of Complex 1-Pt

Materials and Methods

Complex 1-Pt was prepared by stirring a mixture of [Pt{bzimpy(PrSO₃)₂}Cl]PPN (100 mg, 0.076 mmol), HC≡C—C₆H₃—(CH₂OH)₂-3.5 (40 mg, 0.247 mmol), copper(I) iodide (catalytic amount), and triethylamine (1 mL) in degassed methanol (100 mL) under nitrogen at 100° C. for 1 day. Subsequent to removal of the solvent by rotary evaporation, the solid residue was dissolved in methanol. After filtration, the complex was then recrystallized from a diethyl ether-methanol solution. The final water-soluble complex was then prepared by salt metathesis reaction with potassium hexafluorophosphate. The precipitate was isolated by centrifugation and washed successively with acetonitrile and dichloromethane. The final product was obtained as an orange solid.

Proton nuclear magnetic resonance (¹H NMR) spectra were recorded on a Bruker AVANCE 400 Fourier Transform NMR Spectrometer (400 MHz) with tetramethylsilane as an internal standard. Infrared (IR) spectra were obtained on a KBr disk from a Shimadzu IRAffinity-1 Fourier Transform Infrared Spectrophotometer (7800-350 cm⁻¹). Negative fast atom bombardment (FAB) mass spectra were recorded on a Thermo Fisher Scientific DFS High Resolution Magnetic Sector Mass Spectrometer. Elemental analyses were carried out on a Thermo Fisher Scientific Flash EA 1112 Elemental Analyzer at the Institute of Chemistry, Chinese Academy of Sciences.

Results

The chemical characterization data of complex 1-Pt are as follows.

Yield: 40 mg (56%). ¹H NMR (400 MHz, [D₆]DMSO, 298 K, δ/ppm): δ 2.17 (m, 4H, —CH₂—), 2.70 (t, J=6.3 Hz, 4H, —CH₂SO₃), 4.60 (d, J=5.7 Hz, 4H, —CH₂O), 4.88 (m, 4H, —CH₂N—), 5.42 (t, J=6.0 Hz, 2H, —OH), 7.18 (s, 1H, phenyl), 7.23 (s, 2H, phenyl), 7.51 (m, 4H, benzimidazolyl), 7.83 (m, 2H, benzimidazolyl), 8.15 (t, J=8.2 Hz, 1H, pyridyl), 8.39 (m, 2H, benzimidazolyl), 8.69 ppm (d, J=8.2 Hz, 2H, pyridyl). IR (KBr): v=2120 cm⁻¹ (w; v(C≡C)). Negative FAB-MS: m/z 910 [M-K]⁻. Elemental analysis calcd. (%) for C₃₅H₃₂KN₅O₈PtS₂.2CH₂Cl₂: C, 39.72; H, 3.24; N, 6.26. found: C, 39.52; H, 3.16, N, 6.17.

The results of the analyses confirmed the high purity of complex 1-Pt.

### Example 2. Photophysical Properties of Complex 1-Pt

Materials and Methods

The photophysical properties of complex 1-Pt were measured at a concentration of 30 μM. Luminescence quantum yields in degassed DMF and aqueous solutions at 298 K were measured with the optical dilution method reported in Crosby et al., J. Phys. Chem., 75:991-1024 (1971), using degassed acetonitrile and aqueous solutions of [Ru(bpy)₃]Cl₂ as reference, respectively, as described in (1) Van Houten et al., J. Am. Chem. Soc., 98:4853-4858 (1976), (2) Caspar et al., J. Am. Chem. Soc., 105:5583-5590 (1983), and (3) Wallace et al., Inorg. Chem., 32:3836-3843 (1993). The photoexcitation wavelength was 436 nm.

Results

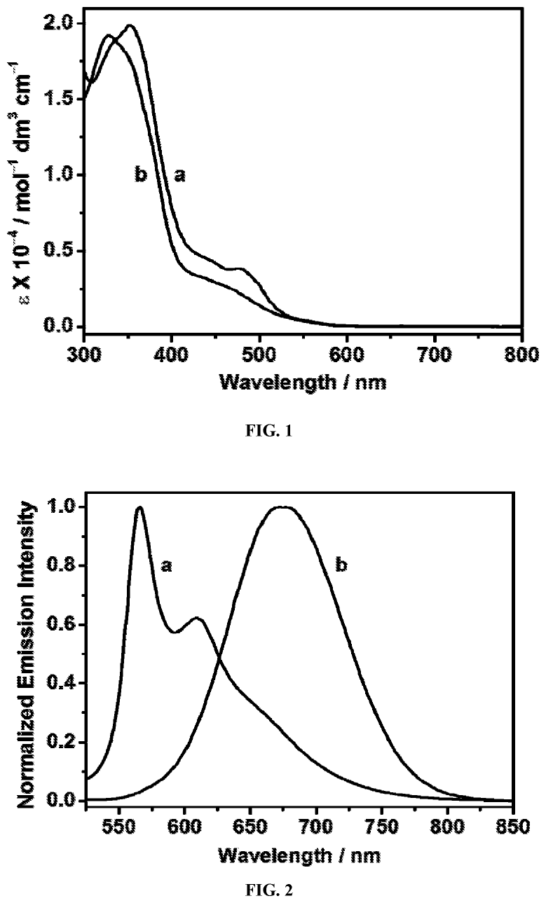

The UV-vis absorption spectra of complex 1-Pt in both DMF and aqueous solutions at 298 K displayed absorption tails at around 470-480 nm, which were attributed to metal-to-ligand charge transfer (MLCT) [dπ(Pt)→π*(bzimpy)] transitions, with some ligand-to-ligand charge transfer (LLCT) [π(C≡C)→π*(bzimpy)] character (FIG. 1). Complex 1-Pt in degassed DMF solution at 298 K exhibited a vibronic-structured emission band at 566 nm (FIG. 2). This emission band was derived from triplet intra-ligand (³IL) [π→π*(bzimpy)] excited state. Complex 1-Pt in degassed aqueous solution at 298 K showed a Gaussian-shape emission band at 673 nm, which was attributed to be originated from triplet metal-metal-to-ligand charge transfer (³MMLCT) excited state.

### Example 3. Insulin Amyloid can Induce Aggregation and Supramolecular Self-Assembly of Complex 1-Pt in Aqueous Solution

Materials and Methods

To induce amyloid fibrillation, insulin was dissolved at 1.0 mg mL⁻¹ in an acidic buffer ([NaCl]=137 mM, [KCl]=2.7 mM, pH=2.0). The solution was incubated at 65° C. and stirred at 300 rpm for 120 minutes to form insulin amyloid. Different amounts of insulin amyloid or native insulin (0-10 μM) were added to a solution of complex 1-Pt (50 μM) in a PBS buffer (10.0 mM, pH=7.4, 10% DMSO). UV-Vis absorption spectra, emission spectra, and RLS spectra were recorded at 25° C. with various amounts of insulin amyloid or native insulin. The emission spectra were recorded at an excitation wavelength of 400 nm.

Results

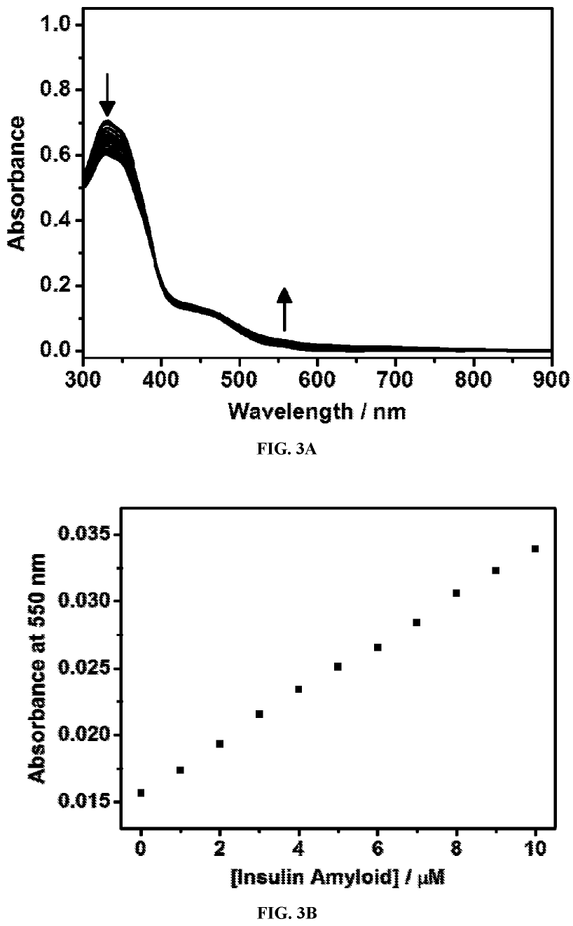

FIG. 3A shows the UV-vis absorption spectra of complex 1-Pt (50 μM) and the corresponding absorbance changes when mixed with increasing amounts of insulin amyloid (0-10 μM). Addition of insulin amyloid to complex 1-Pt gave rise to an increase in absorbance of the low-energy absorption tails at around 550 nm (FIG. 3B), which was due to aggregation and supramolecular self-assembly of the metal complex.

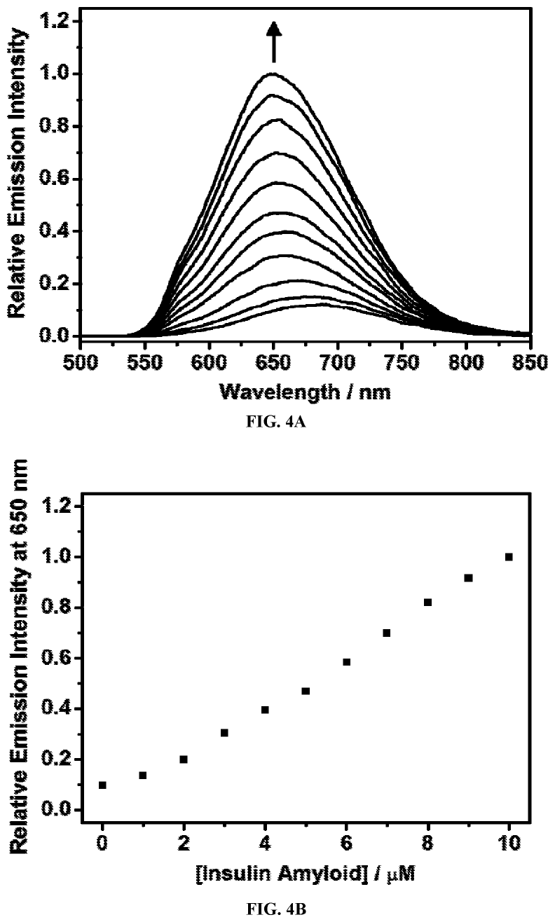

FIG. 4A shows the corrected emission spectra of complex 1-Pt (50 μM) and the corresponding emission intensity changes when mixed with increasing amounts of insulin amyloid (0-10 μM). Addition of insulin amyloid to complex 1-Pt gave rise to a luminescence turn-on at 650 nm (FIG. 4B), which was due to aggregation and supramolecular self-assembly of the metal complex.

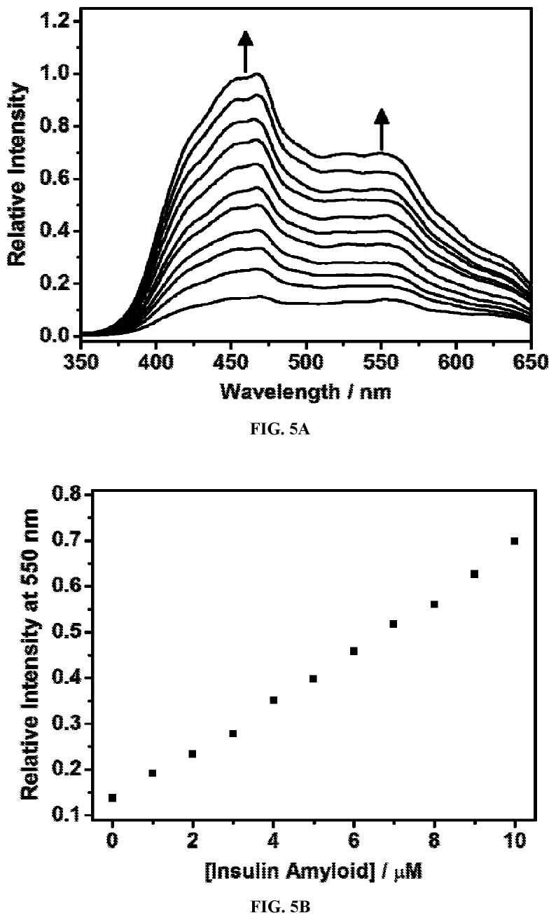

FIG. 5A shows the RLS spectra of complex 1-Pt (50 μM) and the corresponding RLS intensity changes when mixed with increasing amounts of insulin amyloid (0-10 μM). Addition of insulin amyloid to complex 1-Pt gave rise to a remarkable RLS intensity enhancement at around 550 nm (FIG. 5B), which was due to aggregation and supramolecular self-assembly of the metal complex.

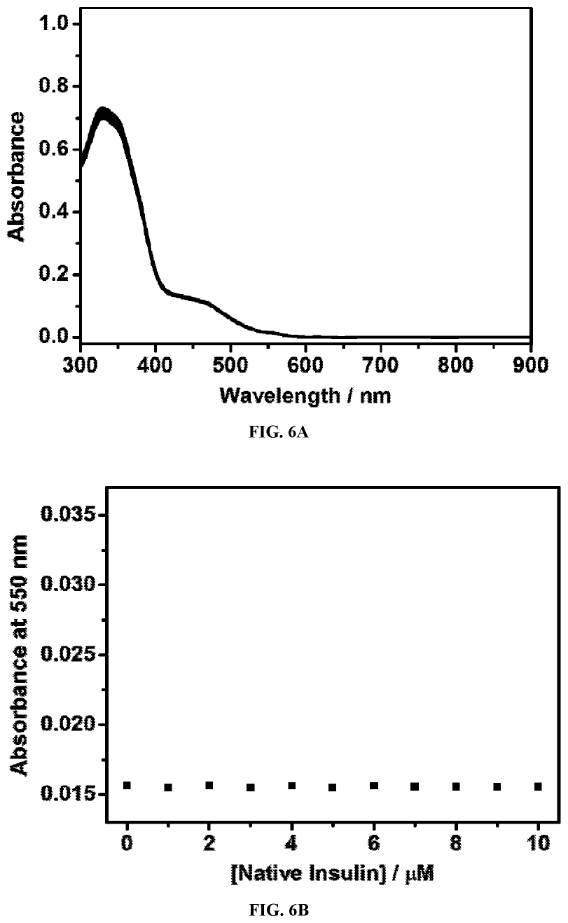

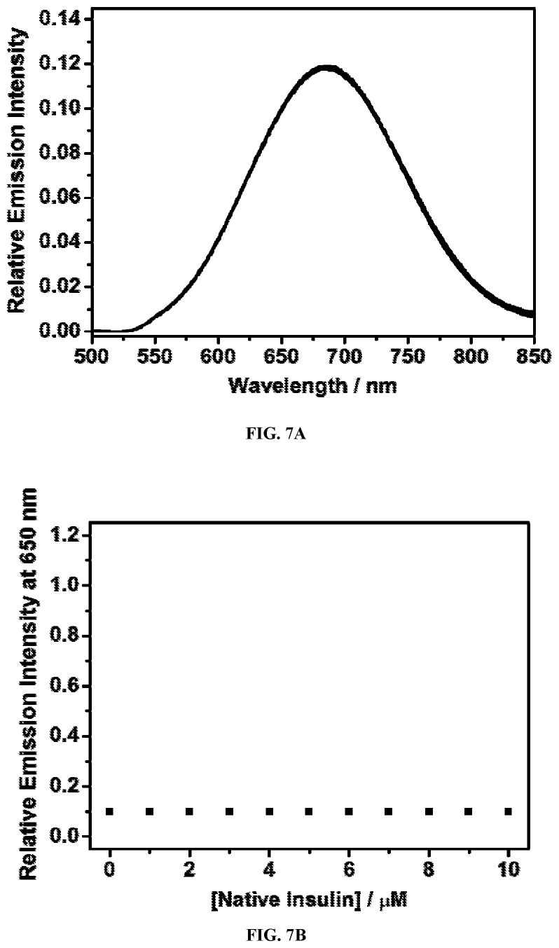

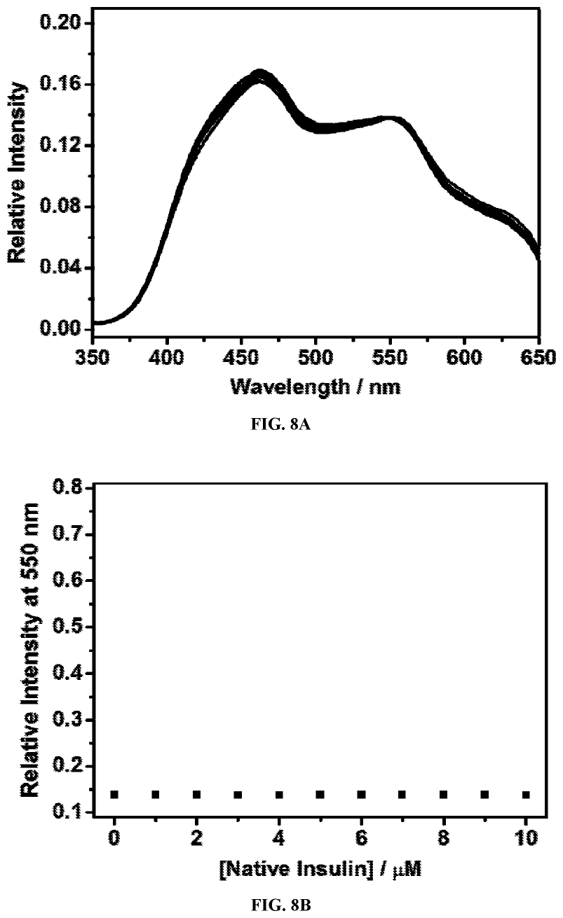

No obvious spectroscopic changes were observed in the UV-vis absorption spectra (FIGS. 6A and 6B), emission spectra (FIGS. 7A and 7B), and RLS spectra (FIGS. 8A and 8B) of complex 1-Pt when mixed with native insulin. These results show that insulin amyloid can induce aggregation and supramolecular self-assembly of complex 1-Pt in aqueous buffer solution, whereas native insulin cannot.

### Example 4. Complex 1-Pt can be Used to Study the Kinetics for Insulin Amyloid Fibrillation

Materials and Methods

To induce amyloid fibrillation, insulin was dissolved at 1.0 mg mL⁻¹ in an acidic buffer ([NaCl]=137 mM, [KCl]=2.7 mM, pH=2.0). The solution was incubated at 65° C. and stirred at 300 rpm. Insulin samples with different incubation times were added to a solution of thioflavin T in a PBS buffer (10.0 mM, pH=7.4). The final concentrations of insulin and thioflavin T were both 10 μM. The emission spectra were recorded at 25° C. at an excitation wavelength of 440 nm. Similarly, insulin samples with different incubation times were added to a solution of complex 1-Pt in a PBS buffer (10.0 mM, pH=7.4, 10% DMSO). The final concentrations of insulin and complex 1-Pt were 10 μM and 50 μM, respectively. UV-Vis absorption spectra, emission spectra, and RLS spectra were recorded at 25° C.; the emission spectra were recorded at an excitation wavelength of 400 nm.

In kinetics, insulin amyloid fibrillation can be modeled by a sigmoid function with three different phases: lag phase, log phase, and stationary phase. The values of kₐₚₚ and t_{lag} for insulin amyloid fibrillation were determined using the sigmoid function which was based on the equation shown below (see similar examples of data fitting in Nielsen et al., Biochemistry, 40:6036-6046 (2001); Hwang et al., J. Biol. Chem., 285:41701-41711 (2010); and Donabedian et al., ACS Chem. Neurosci., 6:1526-1535 (2015)).

$${y}_{y} = \frac{{A}_{1} - {A}_{2}}{1 + {e}^{[ ( x - {x}_{0} ) \text{/} {d}_{x} ]}} + {A}_{2}$$

where y is the absorbance, emission intensity, or RLS intensity; A₁ is the absorbance, emission intensity, or RLS intensity before amyloid fibrillation; A₂ is the absorbance, emission intensity, or RLS intensity after amyloid fibrillation; x is the incubation time; xₒ is the incubation time at which the absorbance, emission intensity, or RLS intensity is at half maximum; and dₓ is the time constant. Accordingly, the apparent rate constant, kₐₚₚ, is equivalent to 1/dₓ, and the lag time, t_{lag}, is given by xₒ−2dₓ.

Results

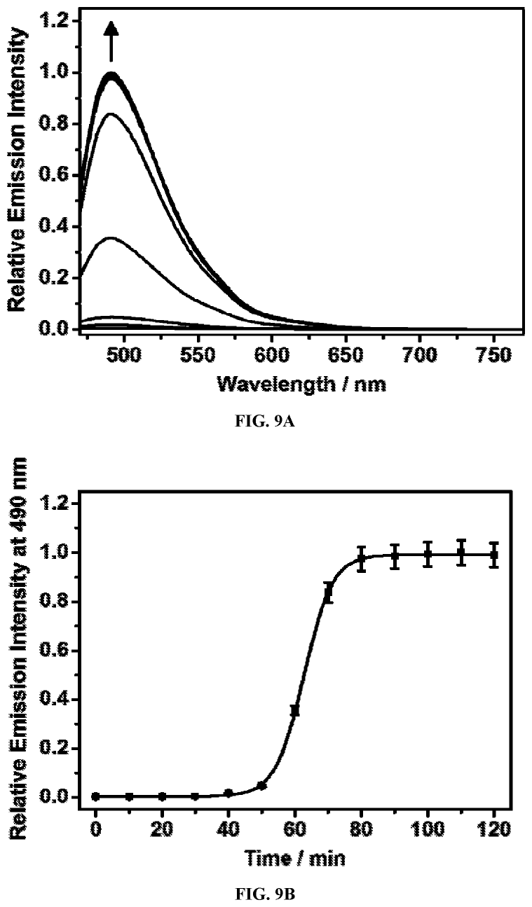

FIG. 9A shows the corrected emission spectra of thioflavin T (10 μM) and the corresponding emission intensity changes when mixed with insulin samples (10 μM) with different incubation times. The formation of amyloid fibrils occurred through a nucleation-dependent mechanism, which is evidenced by the sigmoidal curve (FIG. 9B).

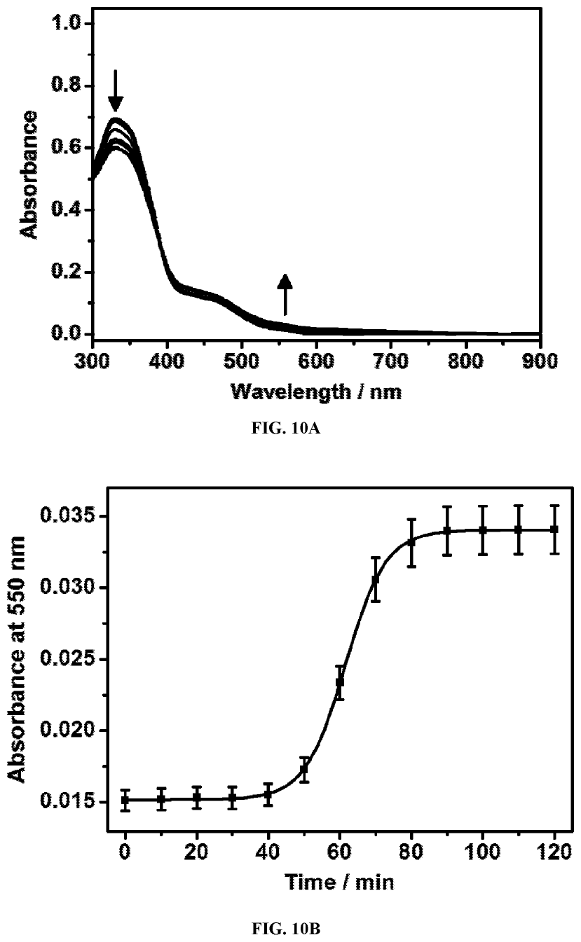

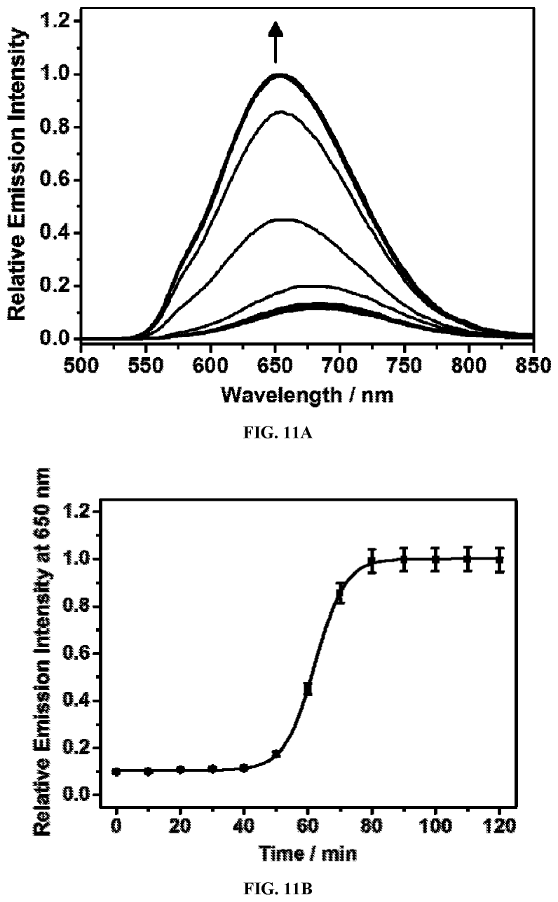

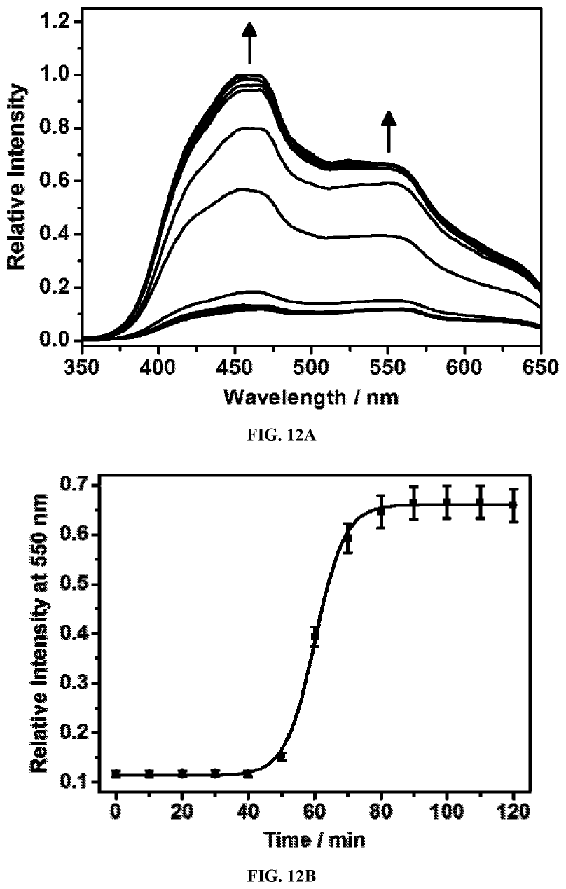

FIGS. 10A, 11A, and 12A show the UV-vis absorption spectra, emission spectra, and RLS spectra, respectively, of complex 1-Pt (50 μM) and the corresponding spectral changes when mixed with insulin samples (10 μM) with different incubation times. All data acquired can be fitted by a sigmoid function (FIGS. 10B, 11B, and 12B). As illustrated in FIG. 13, the kinetic behavior of insulin amyloid fibrillation exhibited three phases: lag phase, log phase, and stationary phase. Over the course of insulin amyloid fibrillation, induced aggregation and supramolecular self-assembly of complex 1-Pt occurred, leading to remarkable changes in its photophysical properties.

The apparent rate constants and lag times calculated for insulin amyloid fibrillation are summarized in Table 1. As shown, the kinetic parameters reported by complex 1-Pt are comparable to those reported by thioflavin T, regardless of the spectroscopic method used for detection.

TABLE 1
Apparent rate constants (kₐₚₚ) and lag times (t_{lag}) of insulin amyloid
fibrillation reported by thioflavin T and complex 1-Pt.

| Probe | Method | kₐₚₚ/min⁻¹ | t_{lag}/min |
|---|---|---|---|
| Thioflavin T | Emission | 0.23 ± 0.08 | 54.0 ± 0.1 |
| Complex 1-Pt | UV-Vis absorption | 0.18 ± 0.09 | 50.2 ± 0.1 |
| Complex 1-Pt | Emission | 0.21 ± 0.10 | 52.7 ± 0.2 |
| Complex 1-Pt | RLS | 0.22 ± 0.11 | 51.1 ± 0.4 |

### Example 5. Complex 1-Pt has a High Binding Affinity to Amyloid and Plaque

Materials and Methods

Different amounts of complex 1-Pt (0-50 μM) were added to a solution of insulin amyloid (10 μM) in a PBS buffer (10.0 mM, pH=7.4, 10% DMSO). The emission spectra were recorded at 25° C. at an excitation wavelength of 400 nm. The experimental data were fit using the Hill equation as shown below (see similar examples of data fitting in Donabedian et al., ACS Chem. Neurosci., 6:1526-1535 (2015); Goutelle et al., Fundam. Clin. Pharmacol., 22: 633-648 (2008); and Gesztelyi et al., Arch. Hist. Exact Sci., 66:427-438 (2012)).

$$y = \frac{{x}^{n}}{{K}_{d} + {x}^{n}}$$

where y is the relative emission intensity; x is the concentration of complex 1-Pt; n is the Hill coefficient which describes the cooperativity of binding to insulin amyloid; and K_{d} is the apparent dissociation constant. The apparent binding constant, Kₐ, is the inverse of K_{d}.

Results

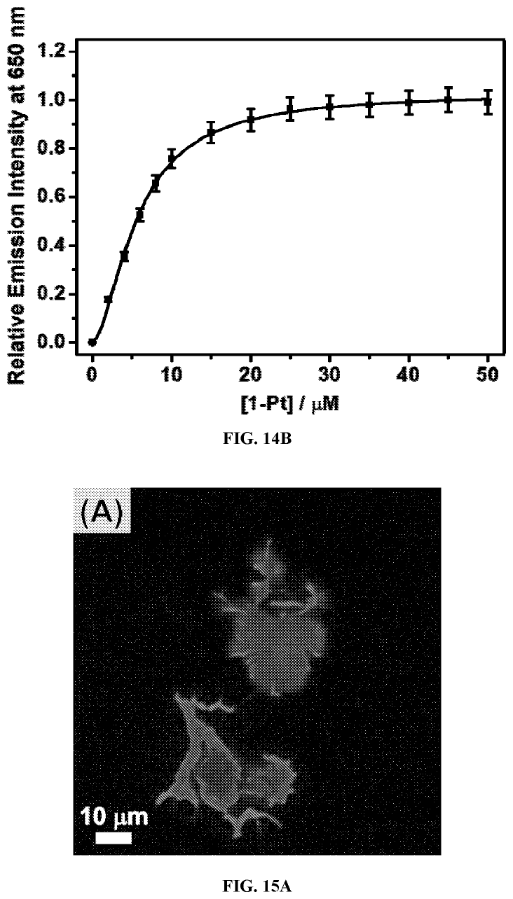

FIG. 14A shows the corrected emission spectra of different concentrations of complex 1-Pt upon addition of the same amount of insulin amyloid. The binding curve acquired was fitted to the Hill equation (FIG. 14B). The apparent binding constant between complex 1-Pt and insulin amyloid was found to be 5.46×10⁴ M⁻¹, which is in the same order of magnitude as that between thioflavin T and insulin amyloid determined under similar assay conditions.

### Example 6. Complex 1-Pt can Become Strongly Luminescent after Binding to Insulin Amyloid

Materials and Methods

Insulin amyloid (10 μM) was added to a solution of complex 1-Pt (50 μM) in a PBS buffer (10.0 mM, pH=7.4, 10% DMSO). The samples for confocal microscopy were prepared by placing an aliquot of the mixture solution onto a microscope slide, followed by placing a cover slip on top. Confocal microscopy experiments were performed on a Carl Zeiss LSM 700 Confocal Scanning Microscope. The confocal images were taken under a 20× objective using a solid-state laser with an excitation wavelength of 555 nm, and emission was collected at 600-700 nm.

Results

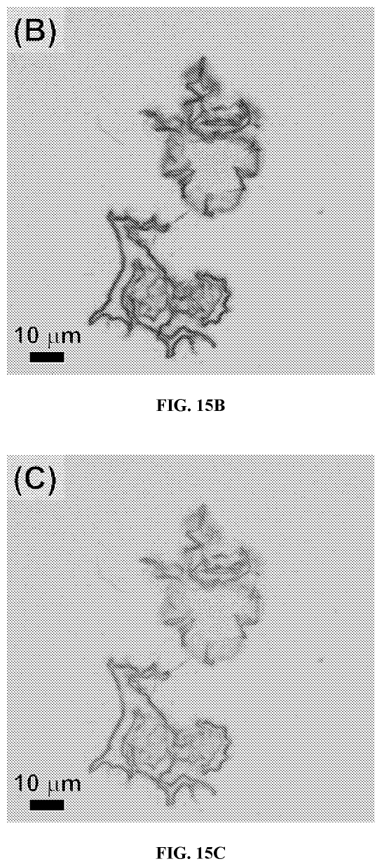

A comparison of the images obtained from laser excitation and bright-field illumination showed that the luminescence of complex 1-Pt was observed only in the regions where amyloid fibrils were present (FIG. 15). Therefore, it can be concluded that aggregation of complex 1-Pt occurred upon binding to insulin amyloid, thereby rendering the metal complex strongly luminescent.

### Example 7. Complex 1-Pt can be Used to Screen Inhibitors Against Protein Aggregation

Materials and Methods

Insulin was dissolved at 1.0 mg mL⁻¹ in an acidic buffer ([NaCl]=137 mM, [KCl]=2.7 mM, pH=2.0) in the presence of different amounts (0, 10, 20, 50, 70, 100 mM) of L-ascorbic acid. The solution was incubated at 65° C. and stirred at 300 rpm. Aliquots were withdrawn at a desired time interval. Insulin samples (10 μM) with different incubation times were added to a solution of thioflavin T (10 μM) in a PBS buffer (10.0 mM, pH=7.4). The emission spectra were recorded at 25° C. at an excitation wavelength of 440 nm. Similarly, insulin samples (10 μM) with different incubation times were added to a solution of complex 1-Pt (50 μM) in a PBS buffer (10.0 mM, pH=7.4, 10% DMSO). The emission spectra were recorded at 25° C. at an excitation wavelength of 400 nm. The values of kₐₚₚ and t_{lag} for insulin amyloid fibrillation were determined from fitting using the sigmoid equation listed above.

Results

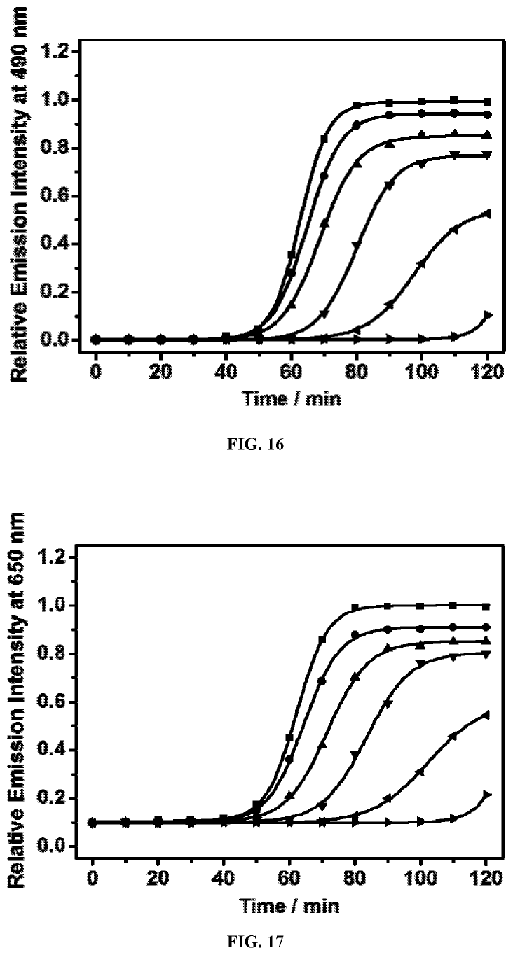

The effect of L-ascorbic acid on insulin amyloid fibrillation was examined by the thioflavin T fluorescence assay as well as the complex 1-Pt luminescence assay. FIG. 16 shows the changes in the relative emission intensity at 490 nm of thioflavin T at different incubation times in the presence of different concentrations of L-ascorbic acid. FIG. 17 shows the changes in the relative emission intensity at 650 nm of complex 1-Pt at different incubation times in the presence of different concentrations of L-ascorbic acid. The apparent rate constants and lag times calculated for insulin amyloid fibrillation in the presence of different concentrations of L-ascorbic acid are summarized in Table 2. It is evident that the two assays yielded very similar results. As shown in Table 2 and the plots of FIGS. 16 and 17, the inhibition effect of L-ascorbic acid on amyloid fibrillation is concentration dependent.

TABLE 2
Apparent rate constants (kₐₚₚ) and lag times (t_{lag}) of
insulin amyloid fibrillation in the presence of different concentrations
of L-ascorbic acid detected by thioflavin T and complex 1-Pt.

| Probe | [L-Ascorbic acid]/mM | kₐₚₚ/min⁻¹ | t_{lag}/min |
|---|---|---|---|
| Thioflavin T | 0 | 0.23 ± 0.08 | 54.0 ± 0.1 |
| | 10 | 0.19 ± 0.09 | 54.3 ± 0.2 |
| | 20 | 0.17 ± 0.08 | 57.2 ± 0.3 |
| | 50 | 0.17 ± 0.08 | 68.3 ± 0.3 |
| | 70 | 0.14 ± 0.07 | 83.0 ± 0.3 |
| | 100 | —^{[a]} | —^{[a]} |
| Complex 1-Pt | 0 | 0.21 ± 0.10 | 52.7 ± 0.2 |
| | 10 | 0.18 ± 0.09 | 53.2 ± 0.2 |
| | 20 | 0.16 ± 0.08 | 59.2 ± 0.3 |
| | 50 | 0.15 ± 0.07 | 70.1 ± 0.6 |
| | 70 | 0.12 ± 0.06 | 85.6 ± 0.6 |
| | 100 | —^{[a]} | —^{[a]} |

^{[a]}Not determined due to the small changes in the relative emission intensities of thioflavin T and complex 1-Pt.

### Example 8. The Performance of Complex 1-Pt Cannot be Interfered Upon Addition of Metal Ions

Materials and Methods

Different metal ions (100 μM), including Mg²⁺, Ca²⁺, Mn²⁺, Fe²⁺, Fe³⁺, Cu²⁺ and Zn²⁺, were individually mixed with complex 1-Pt (50 μM) in a PBS buffer (10.0 mM, pH=7.4, 10% DMSO). To each mixture was added insulin amyloid (10 μM). The emission spectra were recorded at 25° C. at an excitation wavelength of 400 nm.

Results

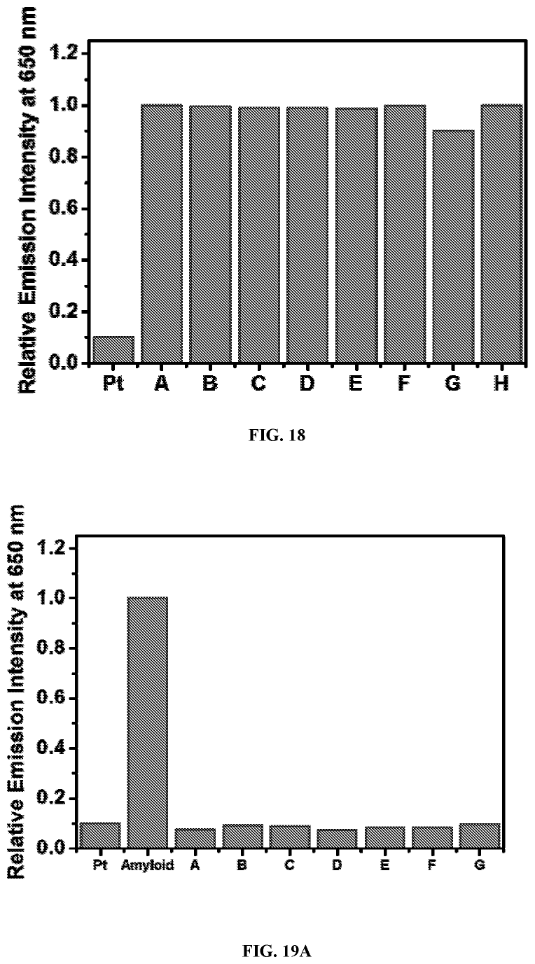

The relative emission intensity of complex 1-Pt was measured in the presence of insulin amyloid and different metal ions. It was found that the relative emission intensity remained almost the same (FIG. 18). This result shows that the amyloid detection performance of complex 1-Pt was not disturbed by the presence of these metal ions, which is one of the most important advantages of this d⁸/d¹⁰ metal complex-based probe over conventionally used probes such as thioflavin T.

### Example 9. The Performance of Complex 1-Pt Cannot be Interfered Upon Addition of Biomolecules

Materials and Methods

Insulin amyloid (10 μM) and/or other biomolecules, including α-amylase (10 μM), albumin from bovine serum (10 μM), albumin from human serum (10 μM), alkaline phosphatase (10 μM), trypsin (10 μM), DNA (10 μg mL⁻¹), and RNA (10 μg mL⁻¹), were added to a solution of complex 1-Pt (50 μM) in a PBS buffer (10.0 mM, pH=7.4, 10% DMSO). The emission spectra were recorded at 25° C. at an excitation wavelength of 400 nm.

Results

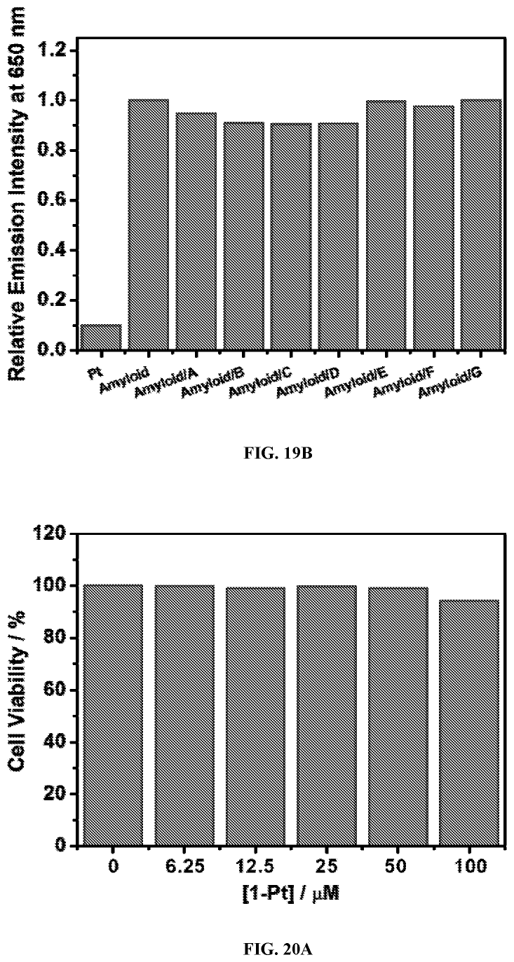

It was found that the emission intensity was enhanced only upon addition of insulin amyloid, with no emission turn-on upon introduction of other biomolecules (FIG. 19A). Simultaneous addition of a mixture of insulin amyloid and the respective interfering biomolecules gave emission intensity changes that were similar to that of adding insulin amyloid alone (FIG. 19B). These results reveal that the present assay was a highly selective and specific sensing platform.

### Example 10. Complex 1-Pt has a Low Cytotoxicity

Materials and Methods

HeLa cells were adhered onto a 96-well plate at around 10,000 cells per well and were cultured with Dulbecco's Modified Eagle medium (DMEM) supplemented with 10% fetal bovine serum (FBS) (100 μL) in a humidified incubator at 37° C. for 24 hours where the carbon dioxide level was kept constant at 5%. Different amounts of complex 1-Pt (0, 6.25, 12.5, 25, 50, 100 μM) in DMEM were applied and the cells were incubated at 37° C. for 24 hours. Similarly, CHO cells were adhered onto a 96-well plate at around 10,000 cells per well and were cultured with Ham's F-12 nutrient mixture supplemented with 10% FBS (100 μL) in a humidified incubator at 37° C. for 24 hours where the carbon dioxide level was kept constant at 5%. Different amounts of complex 1-Pt (0, 6.25, 12.5, 25, 50, 100 μM) in Ham's F-12 nutrient mixture were applied and the cells were incubated at 37° C. for 24 hours. Wells containing cells without complex 1-Pt were used as controls. Subsequently, 10 μL of 3-(4,5-dimethyl-2-thiazolyl)-2,5-diphenyl-2H-tetrazolium bromide (MTT) solution in PBS buffer (5 mg mL⁻¹) was added to each well and the plate was incubated at 37° C. for 3 hours. The solution was removed and the precipitated formazan was dissolved in DMSO (200 μL). After solubilization, the absorbance of formazan at 570 nm was measured with a microplate absorbance reader. The cell viability was expressed as a percentage ratio of the absorbance of the cells treated with complex 1-Pt to that of the controls.

Results

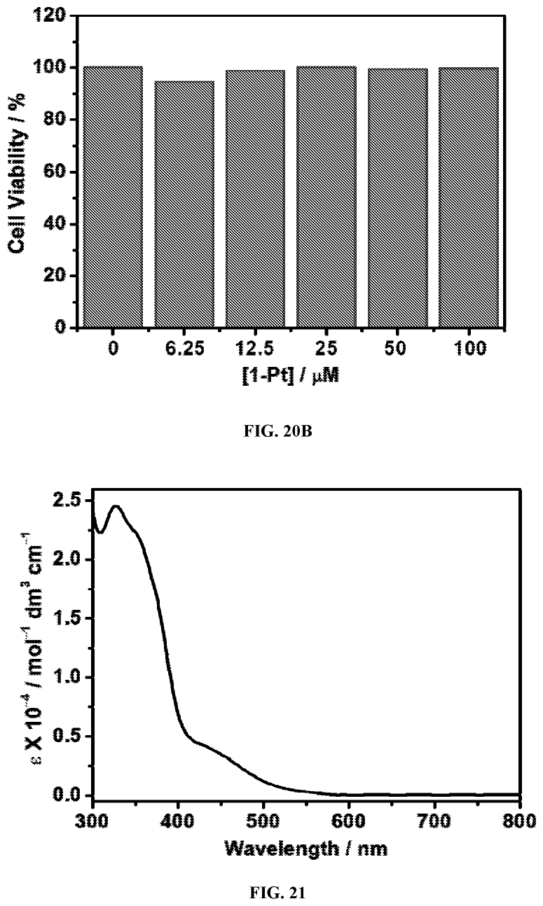

The results show that the HeLa cells maintained over 98% cell viability after the incubation with complex 1-Pt at concentrations up to 50 μM (FIG. 20A). The cell viability was found to decrease slightly when the concentration of complex 1-Pt was raised to 100 μM, which was still above 94%.

On the other hand, the results show that the CHO cells maintained over 94% cell viability after the incubation with complex 1-Pt at concentrations up to 100 μM (FIG. 20B). These results demonstrate the low cytotoxicity of complex 1-Pt.

### Example 11. Synthesis and Characterization of Complex 2-Pt

Materials and Methods

Complex 2-Pt was prepared by stirring a mixture of [Pt{bzimpy(C₄H₉)₂}Cl]Cl (100 mg, 0.145 mmol), [HC≡C—C₆H₄-{NHC(NH₂)(═NH₂)}-4]Cl (100 mg, 0.435 mmol), copper(I) iodide (catalytic amount), and triethylamine (1 mL) in degassed methanol (100 mL) under nitrogen at 100° C. for 1 day. Subsequent to removal of the solvent by rotary evaporation, the solid residue was dissolved in methanol. After filtration, the complex was then recrystallized from a diethyl ether-methanol solution. The precipitate was washed successively with acetonitrile and dichloromethane. The final product was obtained as an orange solid.

Proton nuclear magnetic resonance (¹H NMR) spectra were recorded on a Bruker AVANCE 400 Fourier Transform NMR Spectrometer (400 MHz) with tetramethylsilane as an internal standard. Infrared (IR) spectra were obtained on a KBr disk from a Shimadzu IRAffinity-1 Fourier Transform Infrared Spectrophotometer (7800-350 cm⁻¹). Positive fast atom bombardment (FAB) mass spectra were recorded on a Thermo Fisher Scientific DFS High Resolution Magnetic Sector Mass Spectrometer. Elemental analyses were carried out on a Thermo Fisher Scientific Flash EA 1112 Elemental Analyzer at the Institute of Chemistry, Chinese Academy of Sciences.

Results

The chemical characterization data of complex 2-Pt are as follows.

Yield: 70 mg (57%). ¹H NMR (400 MHz, [D₆]DMSO, 298 K, δ/ppm): δ 0.92 (t, J=7.3 Hz, 6H, —CH₃), 1.44 (m, 4H, —CH₂—), 1.91 (m, 4H, —CH₂—), 4.92 (t, J=7.0 Hz, 4H, —CH₂N—), 7.31 (d, J=8.5 Hz, 2H, phenyl), 7.44 (s, 4H, —NH₂), 7.58 (d, J=8.5 Hz, 2H, phenyl), 7.65 (m, 4H, benzimidazolyl), 8.07 (d, J=8.1 Hz, 2H, benzimidazolyl), 8.53 (d, J=8.1 Hz, 2H, benzimidazolyl), 8.62 (m, 3H, pyridyl), 9.76 ppm (s, 1H, —NH—). IR (KBr): v=2110 cm⁻¹ (w; v(C≡C)). Positive FAB-MS: m/z 389 [M−2Cl]. Elemental analysis calcd. (%) for C₃₆H₃₈Cl₂N₈Pt—CH₂Cl₂: C, 47.60; H, 4.32; N, 12.00. found: C, 47.61; H, 4.63; N, 12.06.

The results of the analyses confirmed the high purity of complex 2-Pt.

### Example 12. Photophysical Properties of Complex 2-Pt

Materials and Methods

The photophysical properties of complex 2-Pt were measured at a concentration of 30 μM. Luminescence quantum yields in degassed methanol and aqueous solutions at 298 K were measured with the optical dilute method reported in Crosby et al., J. Phys. Chem., 75:991-1024 (1971), using degassed acetonitrile and aqueous solutions of [Ru(bpy)₃]Cl₂ as reference, respectively, as described in (1) Van Houten et al., J. Am. Chem. Soc., 98:4853-4858 (1976), (2) Caspar et al., J. Am. Chem. Soc., 105:5583-5590 (1983), and (3) Wallace et al., Inorg. Chem., 32:3836-3843 (1993). The photoexcitation wavelength was 436 nm.

Results

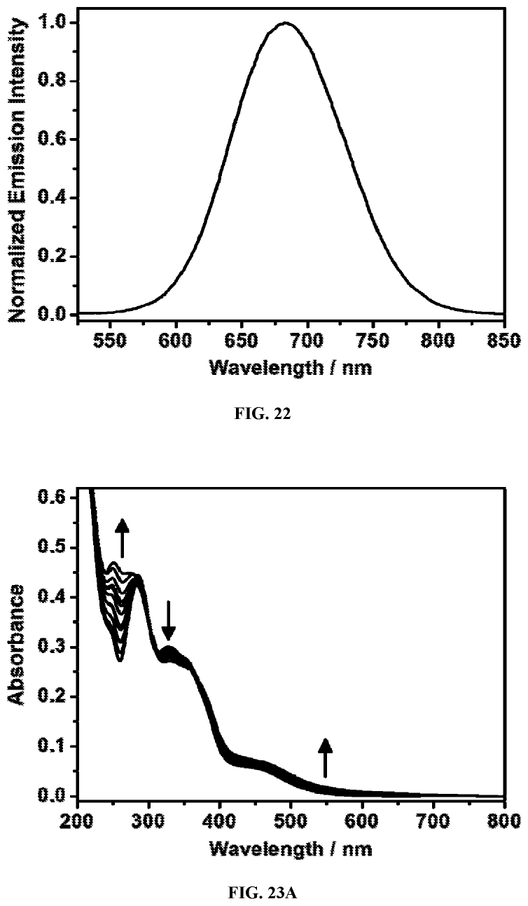

The UV-vis absorption spectra of complex 2-Pt in both methanol and aqueous solutions at 298 K displayed absorption tails at around 450-470 nm which were attributed to metal-to-ligand charge transfer (MLCT) [dπ(Pt)→π*(bzimpy)] transitions, with some ligand-to-ligand charge transfer (LLCT) [π(C≡C)→π*(bzimpy)] character (FIG. 21). Complex 2-Pt in degassed methanol solution at 298 K exhibited a vibronic-structured emission band at 564 nm. This emission band was derived from triplet intra-ligand (³IL) [π→π*(bzimpy)] excited state. Complex 2-Pt in degassed aqueous solution at 298 K showed a Gaussian-shape emission band at 683 nm, which was attributed to be originated from triplet metal-metal-to-ligand charge transfer (³MMLCT) excited state (FIG. 22).

### Example 13. RNA can Induce Aggregation and Supramolecular Self-Assembly of Complex 2-Pt in Aqueous Solution

Materials and Methods

Different amounts of RNA (0-10 μg mL⁻¹) were added to a solution of complex 2-Pt (20 μM) in a PBS buffer (10 mM, pH=7.4). UV-Vis absorption spectra, emission spectra, RLS spectra, and zeta potential data of the samples were recorded at 37° C. The emission spectra were recorded at an excitation wavelength of 360 nm.

Results

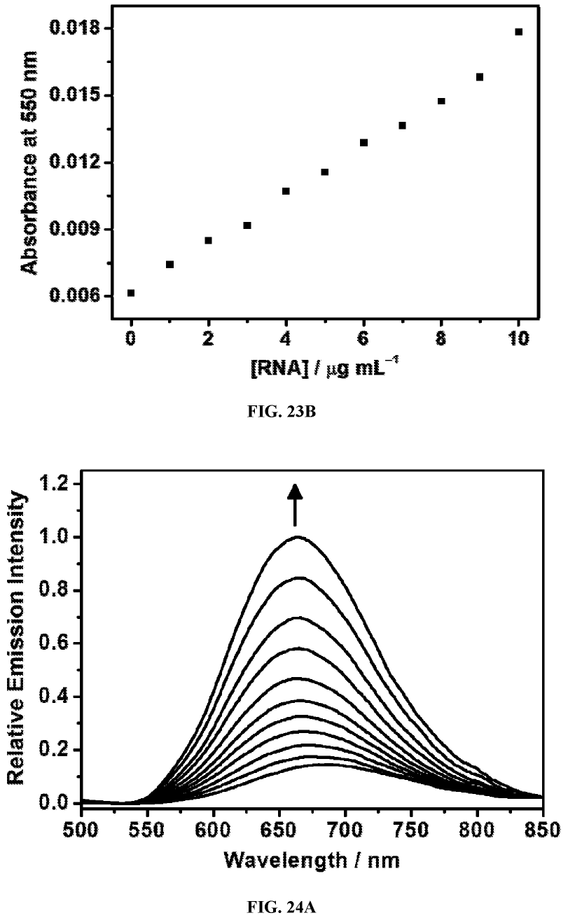

FIG. 23A shows the UV-vis absorption spectra of complex 2-Pt (20 μM) and the corresponding absorbance changes when mixed with increasing amounts of RNA (0-10 μg mL⁻¹). Addition of RNA to complex 2-Pt gave rise to an increase in absorbance of the low-energy absorption tails at around 550 nm (FIG. 23B), which was due to aggregation and supramolecular self-assembly of the metal complex.

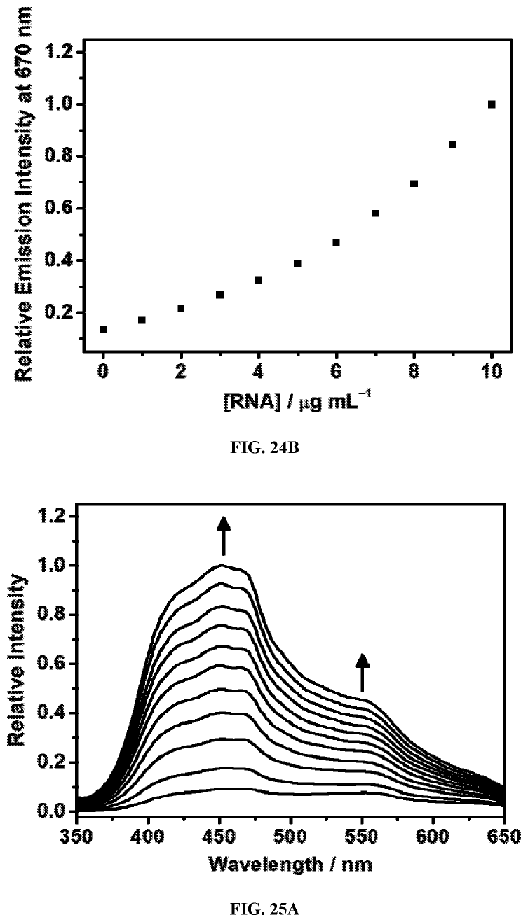

FIG. 24A shows the corrected emission spectra of complex 2-Pt (20 μM) and the corresponding emission intensity changes when mixed with increasing amounts of RNA (0-10 μg mL⁻¹). Addition of RNA to complex 2-Pt gave rise to a luminescence turn-on at 670 nm (FIG. 24B), which was due to aggregation and supramolecular self-assembly of the metal complex.

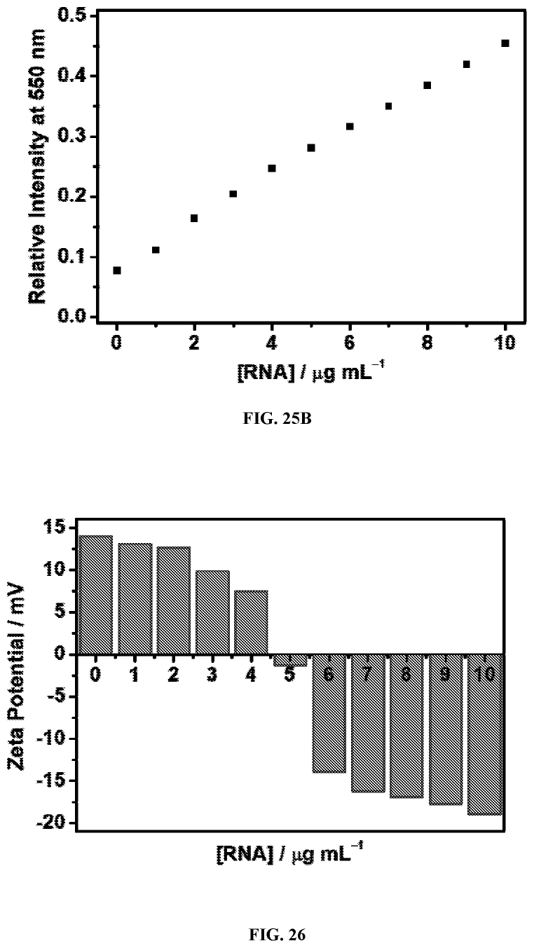

FIG. 25A shows the RLS spectra of complex 2-Pt (20 μM) and the corresponding RLS intensity changes when mixed with increasing amounts of RNA (0-10 μg mL⁻¹). Addition of RNA to complex 2-Pt gave rise to a remarkable RLS intensity enhancement at around 550 nm (FIG. 25B), which was due to aggregation and supramolecular self-assembly of the metal complex.

FIG. 26 shows the zeta potential data of complex 2-Pt (20 μM) upon addition of different amounts of RNA (0-10 μg mL⁻¹) in PBS buffer. Addition of RNA to complex 2-Pt gave rise to a more negative zeta potential, which was due to the binding of the metal complexes with RNA.

### Example 14. Complex 2-Pt has a High Binding Affinity to RNA

Materials and Methods

Different amounts of complex 2-Pt (0-20 μM) were added to a solution of RNA (10 μg mL⁻¹) in a PBS buffer (10.0 mM, pH=7.4). The emission spectra were recorded at 37° C. at an excitation wavelength of 360 nm. The experimental data were fit using the Hill equation as shown below.

$$y = \frac{{x}^{n}}{{K}_{d} + {x}^{n}}$$

where y is the relative emission intensity; x is the concentration of complex 2-Pt; n is the Hill coefficient which describes the cooperativity of binding to RNA; and K_{d} is the apparent dissociation constant. The apparent binding constant, Kₐ, is the inverse of K_{d}.

Results

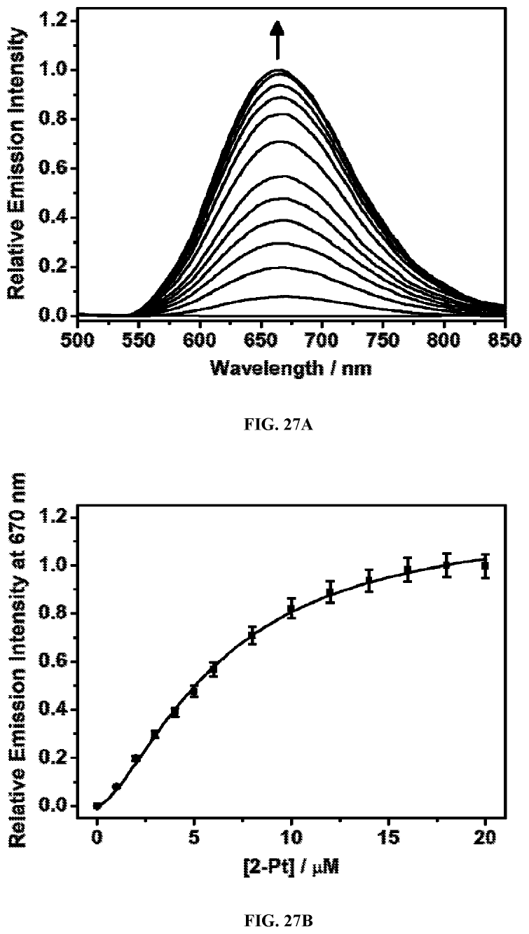

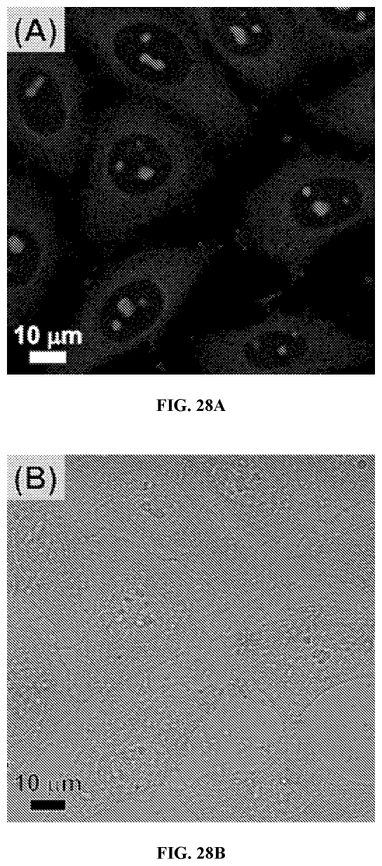

FIG. 27A shows the corrected emission spectra of different concentrations of complex 2-Pt upon addition of the same amount of RNA. The binding curve acquired was fitted to the Hill equation (FIG. 27B). The apparent binding constant between complex 2-Pt and insulin amyloid was found to be 6.01×10⁴ M⁻¹.

### Example 15. Complex 2-Pt can Become Strongly Luminescent after Binding to RNA and Nucleolus

Materials and Methods

HeLa cells were cultured with DMEM supplemented with 10% FBS in a humidified incubator at 37° C. where the carbon dioxide level was kept constant at 5%. Similarly, CHO cells were cultured with Ham's F-12 nutrient mixture supplemented with 10% FBS in a humidified incubator at 37° C. where the carbon dioxide level was kept constant at 5%. The cells were then adhered onto a sterile coverslip in a 35-mm cell culture dish and were cultured for 48 hours. The culture medium was removed and the cells were fixed in pre-chilled methanol at −20° C. for 10 minutes. After the cells were washed in PBS buffer (1 mL) for three times, a solution of complex 2-Pt (20 μM) in PBS buffer was applied and the cells were incubated at 37° C. for 1 hour. After staining, the labeling solution was removed and the cells were washed in PBS buffer (1 mL) for three times. The coverslip was mounted onto a sterile microscope slide. Confocal microscopy experiments were performed on a Leica TCS SPE Confocal Scanning Microscope. The confocal images were taken under a 63× objective using a solid-state laser with an excitation wavelength of 488 nm, and emission was collected at 620-720 nm.

Results

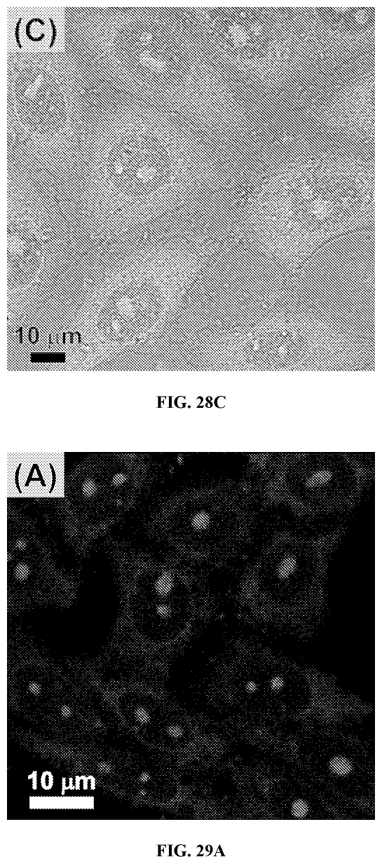

The imaging results show that bright red luminescence spots were clearly visible in the nucleus of the HeLa cells stained by complex 2-Pt (FIGS. 28A-C).

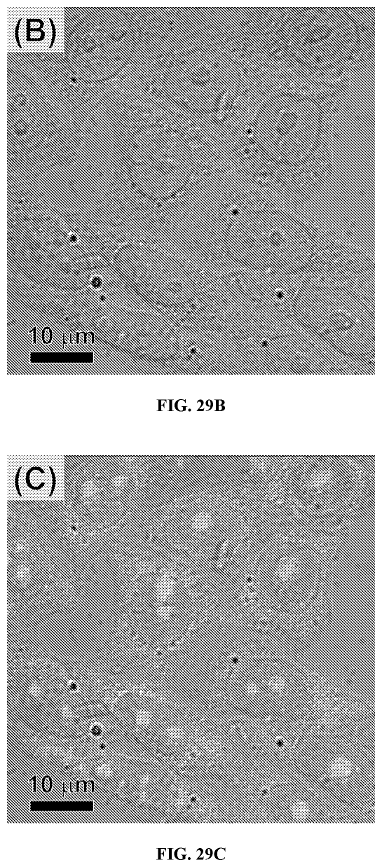

On the other hand, bright red luminescence spots were also clearly visible in the nucleus of the CHO cells stained by complex 2-Pt (FIGS. 29A-C).

FIG. 30 shows the schematic illustration of the design rationale for the luminescence turn-on assay for detection of RNA and nucleolus imaging using a d⁸ or d¹⁰ metal complex. Aggregation and supramolecular self-assembly of the metal complex on RNA induces a luminescence turn-on.

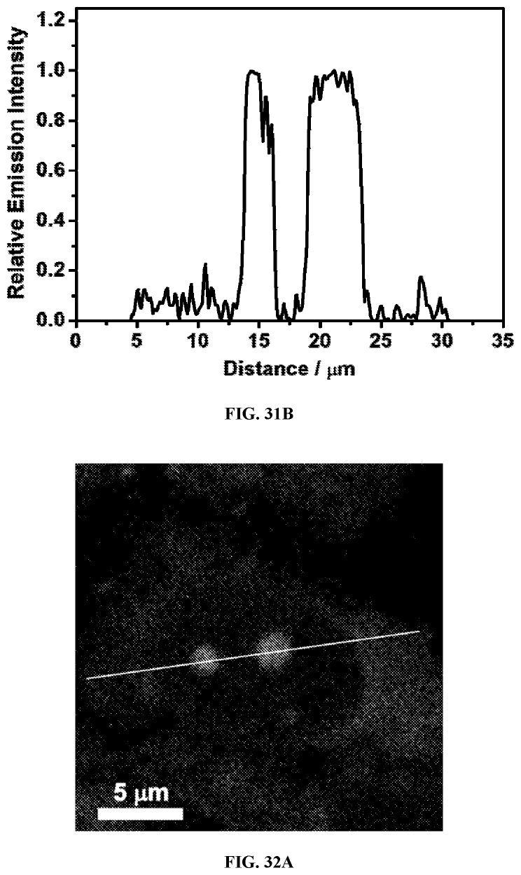

FIG. 31A shows the luminescence confocal image of a fixed HeLa cell stained with complex 2-Pt (20 μM) at 37° C. for 1 hour. FIG. 31B shows the relative emission intensity profile across the fixed HeLa cell from FIG. 31A. The luminescence of complex 2-Pt was found to be mainly in the nucleolus (corresponding to the emission peak between ca. 12 and ca. 25 μm in FIG. 31B), suggesting that selective nucleolus imaging in HeLa cells was achieved by complex 2-Pt.

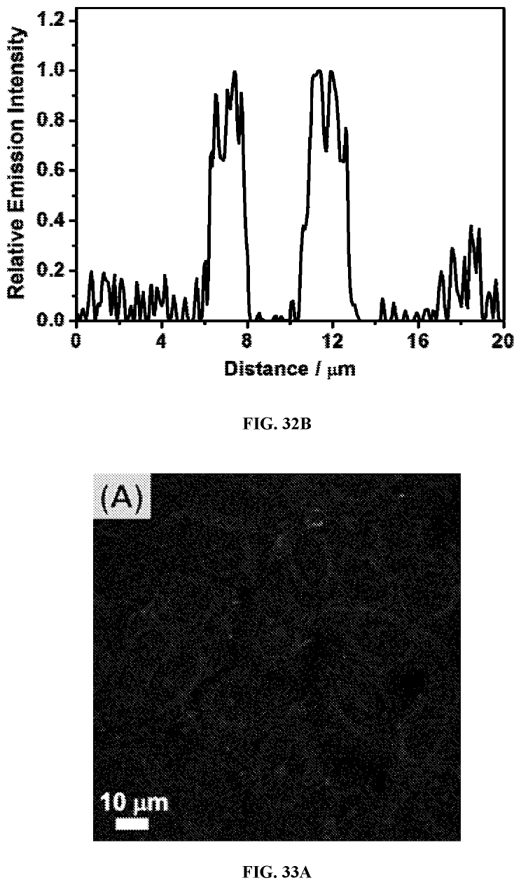

FIG. 32A shows the luminescence confocal image of a fixed CHO cell stained with complex 2-Pt (20 μM) at 37° C. for 1 hour. FIG. 32B shows the relative emission intensity profile across the fixed CHO cell from FIG. 32A. The luminescence of complex 2-Pt was found to be mainly in the nucleolus (corresponding to the emission peak between ca. 7 and ca. 12 μm in FIG. 32B), suggesting that selective nucleolus imaging in CHO cells was achieved by complex 2-Pt.

In addition to the luminescence spots from the cell nuclei, smaller luminescence spots in the HeLa cells and CHO cells were also observed. These smaller luminescence spots represent RNA in the cytoplasm.

### Example 16. Complex 2-Pt can be Used to Detect the Degradation of RNA Catalyzed by RNase

Materials and Methods

After fixed HeLa cells and/or fixed CHO cells were stained with complex 2-Pt (20 μM) at 37° C. for 1 hour, a solution of RNase and/or DNase (30 μg mL⁻¹) in PBS buffer was applied and the cells were incubated at 37° C. for 2 hours. After RNase and/or DNase digestion, the enzyme solution was removed and the cells were washed in PBS buffer (1 mL) for three times. The coverslip was mounted onto a sterile microscope slide. The confocal images were taken under a 63× objective using a solid-state laser with an excitation wavelength of 488 nm, and emission was collected at 620-720 nm.

Results

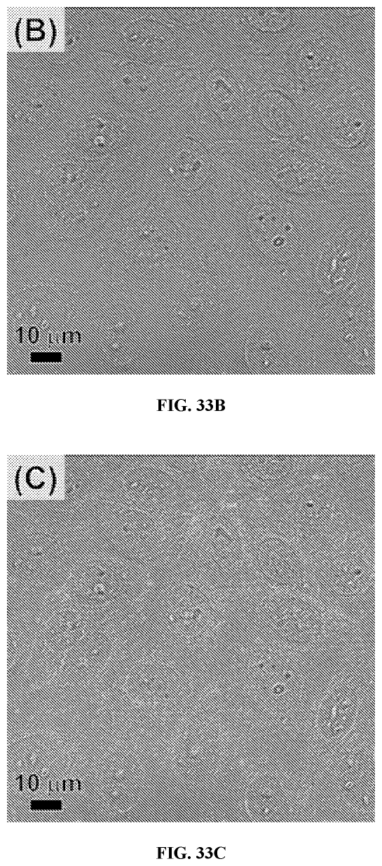

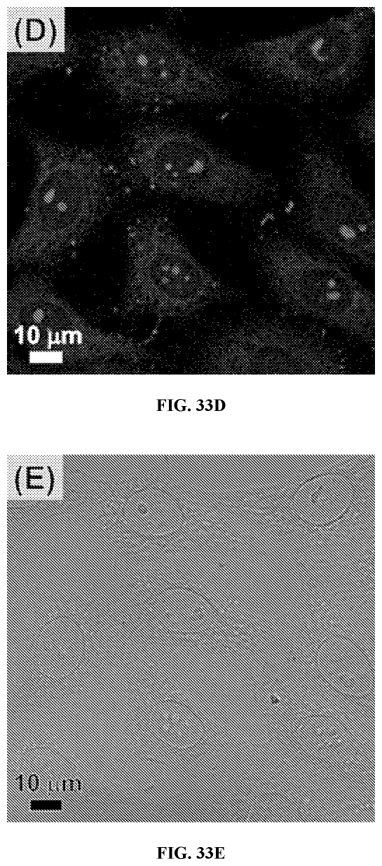

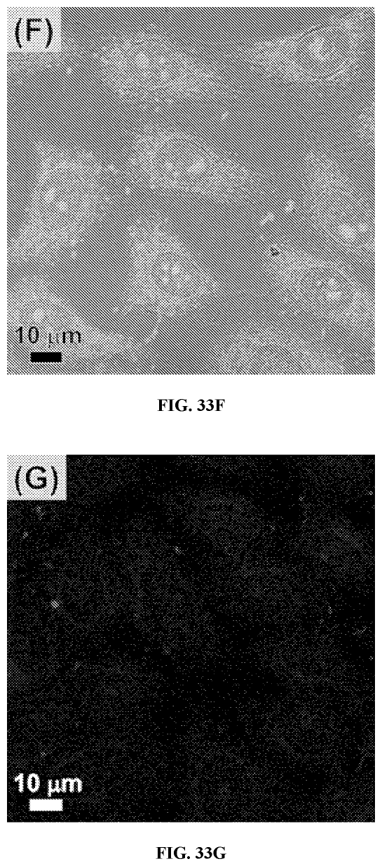

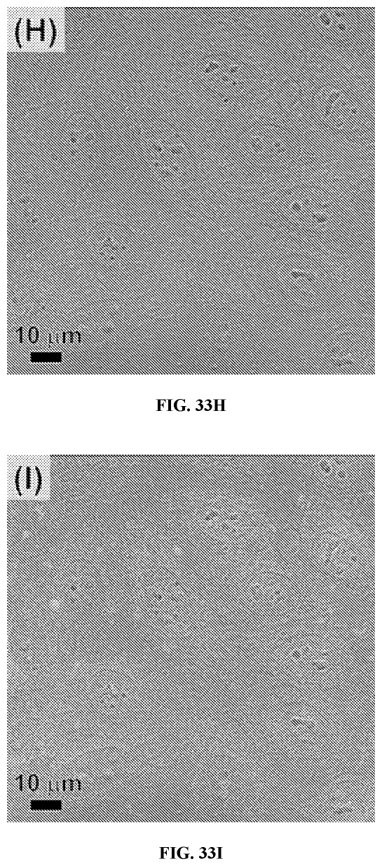

The confocal images of HeLa cells show that upon treatment with RNase, the red luminescence signal of the nucleolus was almost completely lost (FIGS. 33A-33C). In contrast, treatment with DNase caused no significant loss of the red luminescence signal of the nucleolus (FIGS. 33D-33F). Simultaneous treatment with both RNase and DNase (both at 30 μg mL⁻¹) also led to a near complete loss of the red luminescence signal of the nucleolus (FIGS. 33G-33I).

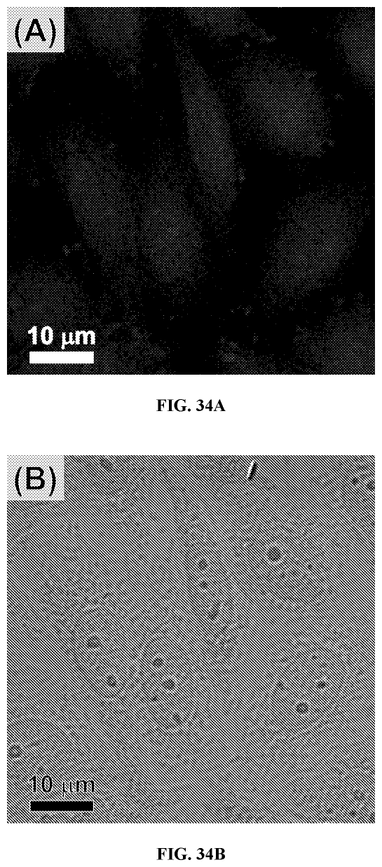

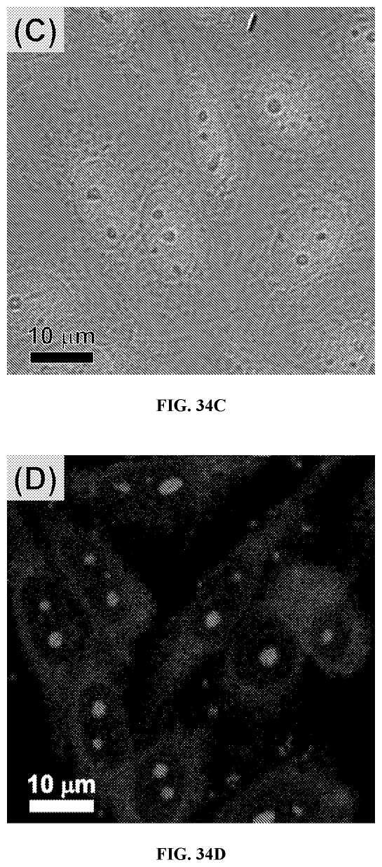

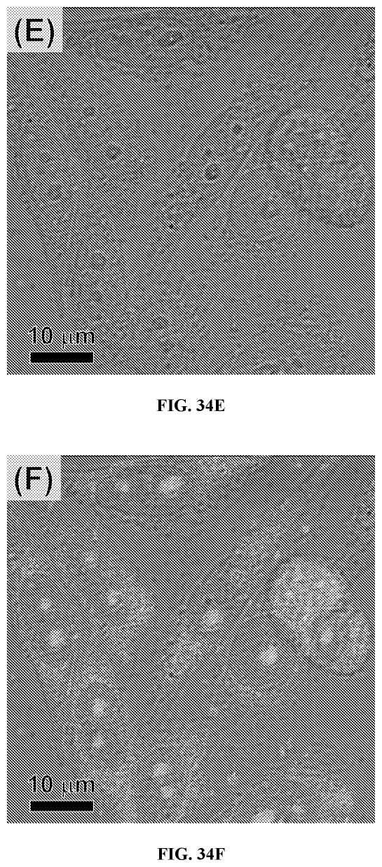

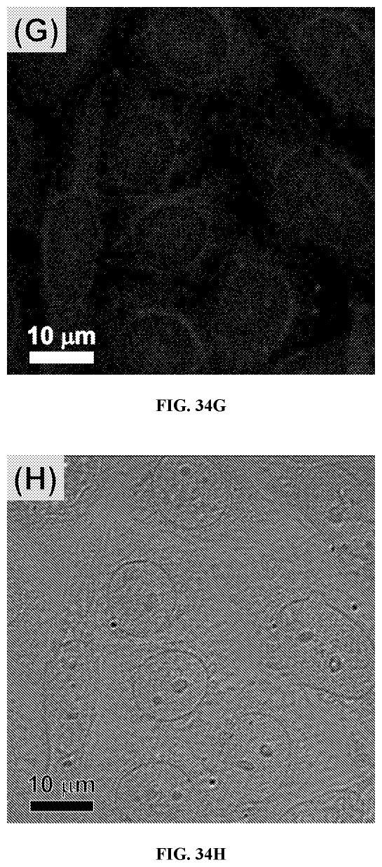

On the other hand, the confocal images of CHO cells show that upon treatment with RNase, the red luminescence signal of the nucleolus was almost completely lost (FIGS. 34A-34C). In contrast, treatment with DNase caused no significant loss of the red luminescence signal of the nucleolus (FIGS. 34D-34F). Simultaneous treatment with both RNase and DNase (both at 30 μg mL⁻¹) also led to a near complete loss of the red luminescence signal of the nucleolus (FIGS. 34G-34I). These results indicate that the luminescence turn-on assay with complex 2-Pt is specific for RNA detection, not DNA detection.

### Example 17. Complex 2-Pt can be Used to Selectively Stain RNA and Nucleolus

Materials and Methods

After fixed HeLa cells and/or fixed CHO cells were stained with complex 2-Pt (20 μM) at 37° C. for 1 hour, a solution of SYTO™ RNASelect™ green fluorescent cell stain (500 nM) in PBS buffer was applied and the cells were incubated at 37° C. for 20 minutes. After staining, the labeling solution was removed and the cells were washed in PBS buffer (1 mL) for three times. The coverslip was mounted onto a sterile microscope slide. The confocal images were taken under a 63× objective using a solid-state laser with an excitation wavelength of 488 nm, and emission was collected at 620-720 nm for complex 2-Pt and 505-555 nm for SYTO™ RNASelect™ green fluorescent cell stain.

Results

Fixed HeLa cells and/or fixed CHO cells were co-stained with complex 2-Pt and the SYTO™ RNASelect™ green fluorescent cell stain, which is a commercially available nucleolus-targeting probe.

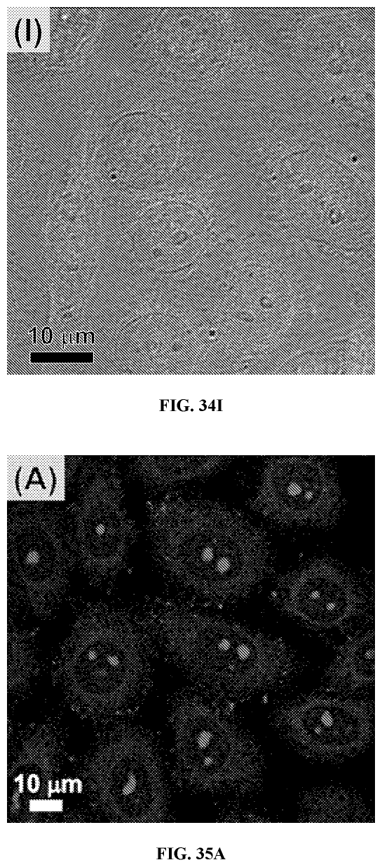

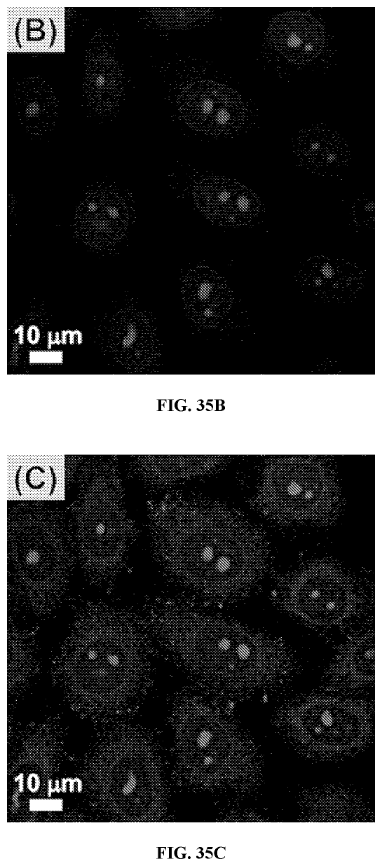

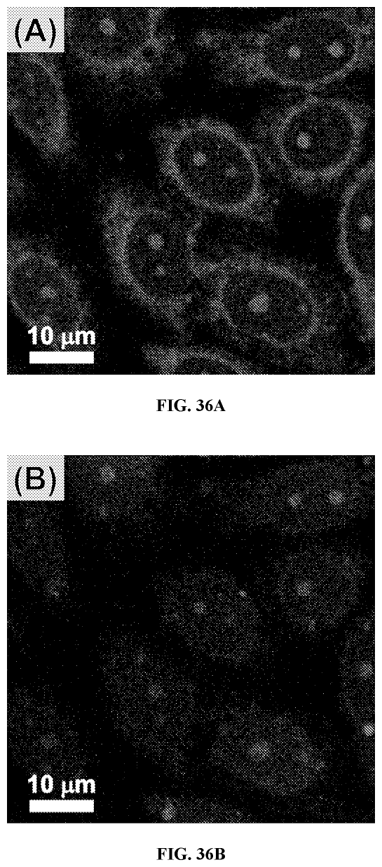

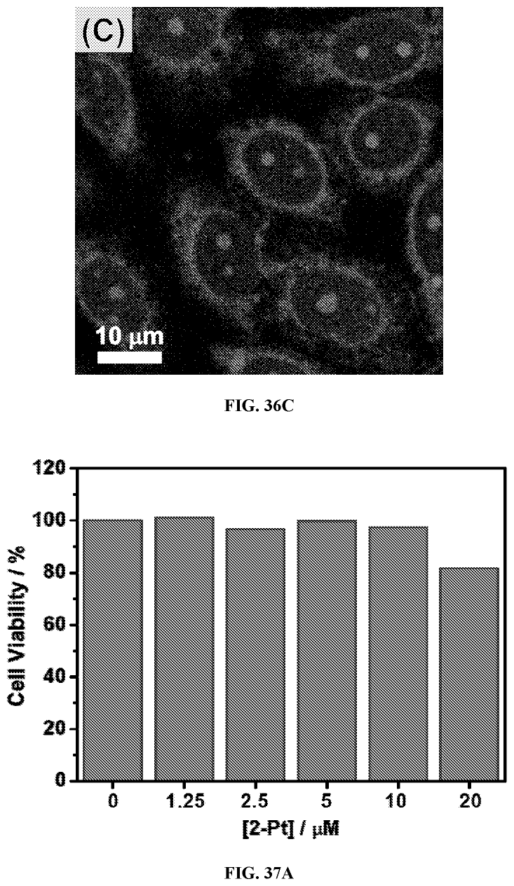

The confocal images of HeLa cells (FIG. 35) and CHO cells (FIG. 36) show that the red luminescence from complex 2-Pt exhibited good co-localization with the green emission from the SYTO™ RNASelect™ green fluorescent cell stain in the nucleolus.

### Example 18. Complex 2-Pt has a Low Cytotoxicity

Materials and Methods

HeLa cells were adhered onto a 96-well plate at around 10,000 cells per well and were cultured with DMEM supplemented with 10% FBS (100 μL) in a humidified incubator at 37° C. for 24 hours where the carbon dioxide level was kept constant at 5%. Different amounts of complex 2-Pt (0, 1.25, 2.5, 5, 10, 20 μM) in DMEM were applied and the cells were incubated at 37° C. for 24 hours. Similarly, CHO cells were adhered onto a 96-well plate at around 10,000 cells per well and were cultured with Ham's F-12 nutrient mixture supplemented with 10% FBS (100 μL) in a humidified incubator at 37° C. for 24 hours where the carbon dioxide level was kept constant at 5%. Different amounts of complex 2-Pt (0, 1.25, 2.5, 5, 10, 20 μM) in Ham's F-12 nutrient mixture were applied and the cells were incubated at 37° C. for 24 hours. Wells containing cells without complex 2-Pt were used as controls. Subsequently, 10 μL of MTT solution in PBS buffer (5 mg mL⁻¹) was added to each well and the plate was incubated at 37° C. for 3 hours. The solution was removed and the precipitated formazan was dissolved in DMSO (200 μL). After solubilization, the absorbance of formazan at 570 nm was measured with a microplate absorbance reader. The cell viability was expressed as a percentage ratio of the absorbance of the cells treated with complex 2-Pt to that of the controls.

Results

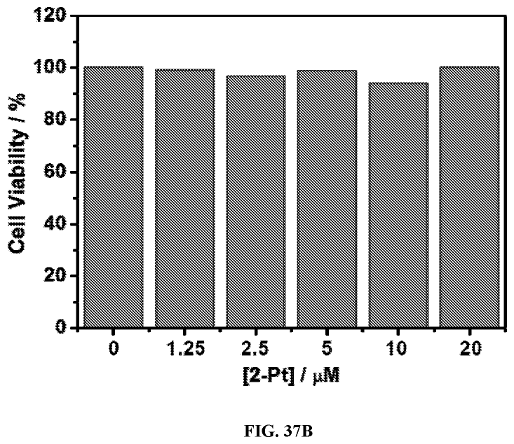

The results show that the HeLa cells maintained over 96% cell viability after the incubation with complex 2-Pt at concentrations up to 10 μM (FIG. 37A). The cell viability was found to decrease slightly when the concentration of complex 2-Pt was raised to 20 μM, which was still above 81%.

On the other hand, the results show that the CHO cells maintained over 94% cell viability after the incubation with complex 2-Pt at concentrations up to 20 μM (FIG. 37B). These results demonstrate the low cytotoxicity of complex 2-Pt.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A compound for detecting and/or imaging an analyte, wherein the compound is a d⁸ or d¹⁰ metal complex or a salt thereof, comprising:
(a) a metal atom with a coordination number of 2, 3, or 4, selected from the group consisting of Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), and Hg(II); and
(b) one or more ligands with donor atoms independently selected from the group consisting of carbon (C), nitrogen (N), oxygen (O), phosphorus (P), sulfur (S), arsenic (As), and selenium (Se),
wherein the metal complex binds to the analyte, wherein binding of the metal complex to the analyte induces aggregation and supramolecular self-assembly of the metal complex through noncovalent metal-metal interactions.

2. The compound of claim 1, wherein the compound has a structure of Formula I: 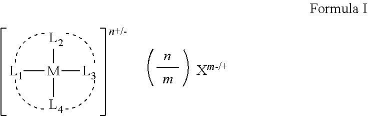
wherein
(a) M represents a metal atom selected from Pt(II), Pd(II), Ni(II), Ir(I), Rh(I), Au(III), Ag(III), and Cu(III),
(b) L₁, L₂, L₃, and L₄ represent ligands, wherein each ligand provides one donor atom to coordinate to the metal atom,
(c) n+/− represents the number of positive or negative charges carried by the metal complex in the formula, wherein n is zero or a positive integer,
(d) X^{m-/+} represents a counterion to maintain charge neutrality, wherein X^{-/+} has a charge opposite to the charge of the metal complex and wherein m is zero or a positive integer, m=n or m≠n,
$$( e ) \frac{n}{m}$$
represents the stoichiometry of the counterion in the formula,
(f) dashed lines represent optional covalent linkages between the two ligands, optional fusion of ring moieties from the two ligands, or a combination thereof.

3. The compound of claim 2, wherein L₁, L₂, and L₃ are optionally substituted, and/or optionally deprotonated C₆-C₅₀ arenes or C₃-C₅₀ heteroarenes, comprising benzene, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, anthracene, pyrene, triazole, tetrazole, pyran, thiapyran, oxadiazole, triazine, tetrazine, carbazole, dibenzothiophene, dibenzofuran, fluorene, and derivatives thereof.

4. The compound of claim 2, wherein L₁ and L₂ are connected by covalent linkages, fusion of ring moieties from the two ligands, or a combination thereof.

5. The compound of claim 1, wherein the compound has a structure of Formula II: 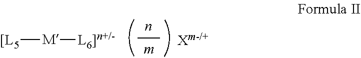
wherein M′ represents a metal atom selected from Ni(0), Pd(0), Pt(0), Cu(I), Ag(I), Au(I), Zn(II), Cd(II), and Hg(II),
wherein L₅ and L₆ represent ligands, wherein each ligand provides one donor atom to coordinate to the metal atom.

6. The compound of claim 1, wherein the compound has a structure of Formula III: 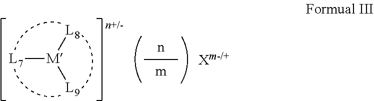
wherein L₇, L₈, and L₉ represent ligands, wherein each ligand provides one donor atom to coordinate to the metal atom.

7. The compound of claim 1, wherein the metal complex binds to the analyte via noncovalent interactions, wherein the noncovalent interactions comprise electrostatic interactions, hydrogen bonding interactions, hydrophobic interactions, or combinations thereof.

8. The compound of claim 1, wherein the metal complex has a planar structure or a partially planar structure.

9. The compound of claim 1, wherein the aggregation and supramolecular self-assembly of the metal complex creates one or more changes in the photophysical properties of the metal complex.

10. The compound of claim 9, wherein the changes in the photophysical properties comprise a change in optical absorbance, luminescence, resonance light scattering (RLS), or combinations thereof.

11. The compound of claim 10, wherein the change in luminescence comprises an increase in the luminescence quantum yield and/or emission intensity, and/or a shift in emission energy or wavelength.

12. The compound of claim 1, wherein the compound is selected from: 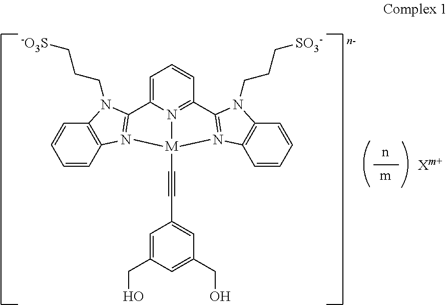
wherein M is Pt(II) (complex 1-Pt), Pd(II) (complex 1-Pd), Ni(II) (complex 1-Ni), Ir(I) (complex 1-Ir), Rh(I) (complex 1-Rh), Au(III) (complex 1-Au), Ag(III) (complex 1-Ag), or Cu(III) (complex 1-Cu),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula; 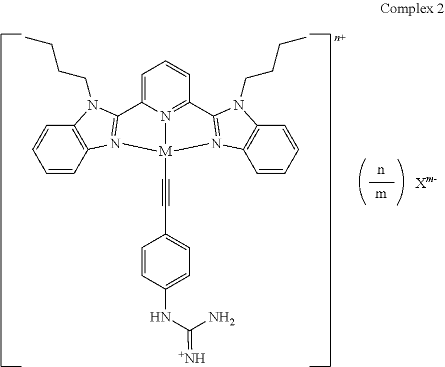
wherein M is Pt(II) (complex 2-Pt), Pd(II) (complex 2-Pd), Ni(II) (complex 2-Ni), Ir(I) (complex 2-Ir), Rh(I) (complex 2-Rh), Au(III) (complex 2-Au), Ag(III) (complex 2-Ag), or Cu(III) (complex 2-Cu),
wherein n+ is the number of positive charges carried by the metal complex in the formula, wherein n is a positive integer,
wherein X^{m-} is a counterion to maintain charge neutrality, wherein m is a positive integer, m=n or m≠n,
wherein
$$\frac{n}{m}$$
is the stoichiometry of the counterion in the formula; 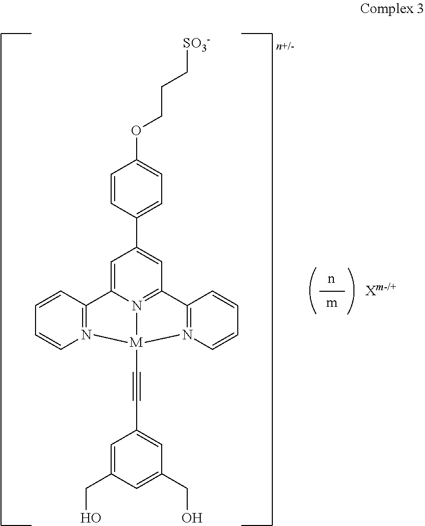
wherein M is Pt(II) (complex 3-Pt), Pd(II) (complex 3-Pd), Ni(II) (complex 3-Ni), Ir(I) (complex 3-Ir), Rh(I) (complex 3-Rh), Au(III) (complex 3-Au), Ag(III) (complex 3-Ag), or Cu(III) (complex 3-Cu),
wherein n+/− is the number of positive or negative charges carried by the metal complex in the formula, wherein n is zero or a positive integer,
wherein X^{m-/+} is a counterion to maintain charge neutrality, wherein m is zero or a positive integer, m=n or m≠n,
wherein
$$\frac{n}{m}$$
is the stoichiometry of the counterion in the formula; 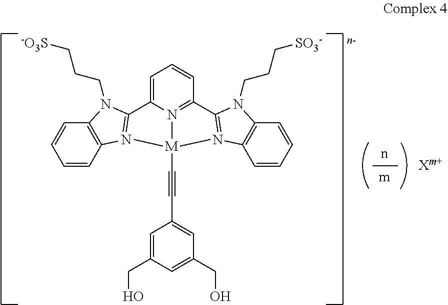
wherein M is Pt(II) (complex 4-Pt), Pd(II) (complex 4-Pd), Ni(II) (complex 4-Ni), Ir(I) (complex 4-Ir), Rh(I) (complex 4-Rh), Au(III) (complex 4-Au), Ag(III) (complex 4-Ag), or Cu(III) (complex 4-Cu);
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 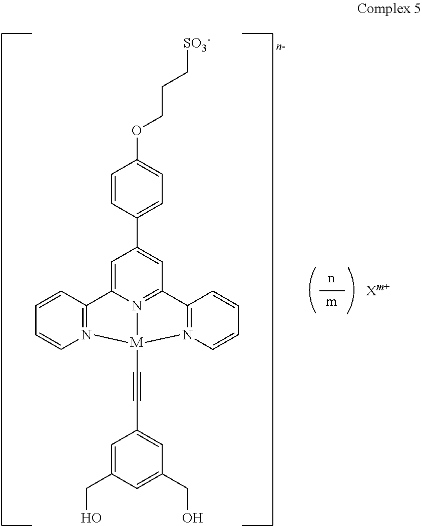
wherein M is Pt(II) (complex 5-Pt), Pd(II) (complex 5-Pd), Ni(II) (complex 5-Ni), Ir(I) (complex 5-Ir), Rh(I) (complex 5-Rh), Au(III) (complex 5-Au), Ag(III) (complex 5-Ag), or Cu(III) (complex 5-Cu),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula; 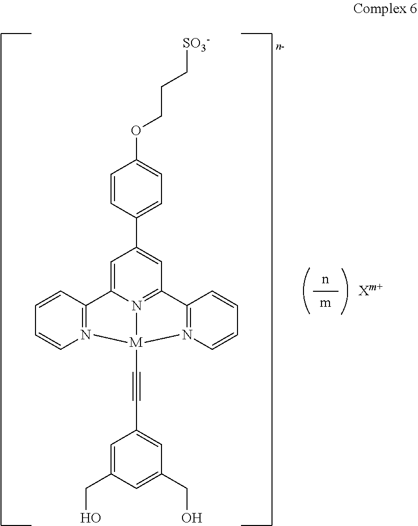
wherein M is Pt(II) (complex 6-Pt), Pd(II) (complex 6-Pd), Ni(II) (complex 6-Ni), Ir(I) (complex 6-Ir), Rh(I) (complex 6-Rh), Au(III) (complex 6-Au), Ag(III) (complex 6-Ag), or Cu(III) (complex 6-Cu),
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 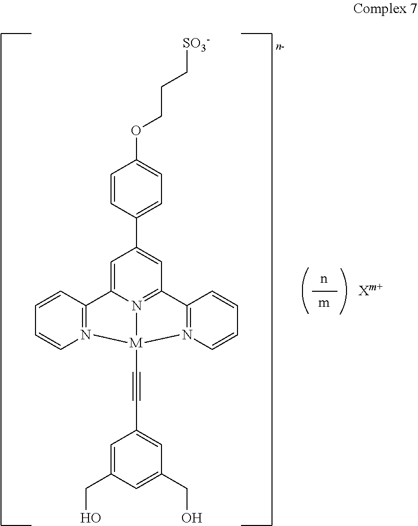
wherein M is Pt(II) (complex 7-Pt), Pd(II) (complex 7-Pd), Ni(II) (complex 7-Ni), Ir(I) (complex 7-Ir), Rh(I) (complex 7-Rh), Au(III) (complex 7-Au), Ag(III) (complex 7-Ag), or Cu(III) (complex 7-Cu),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula; 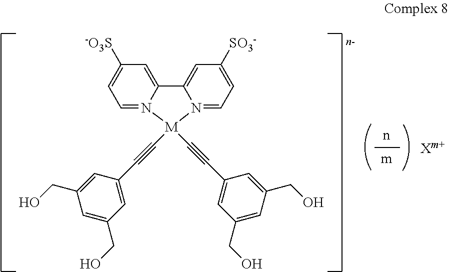
wherein M is Pt(II) (complex 8-Pt), Pd(II) (complex 8-Pd), Ni(II) (complex 8-Ni), Ir(I) (complex 8-Ir), Rh(I) (complex 8-Rh), Au(III) (complex 8-Au), Ag(III) (complex 8-Ag), or Cu(III) (complex 8-Cu),
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 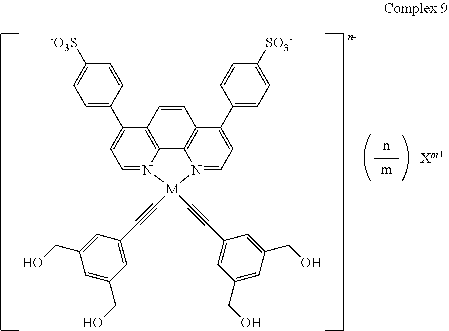
wherein M is Pt(II) (complex 9-Pt), Pd(II) (complex 9-Pd), Ni(II) (complex 9-Ni), Ir(I) (complex 9-Ir), Rh(I) (complex 9-Rh), Au(III) (complex 9-Au), Ag(III) (complex 9-Ag), or Cu(III) (complex 9-Cu),
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 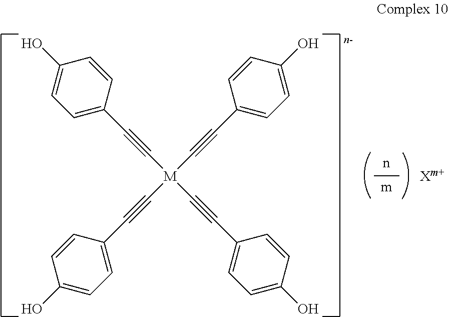
wherein M is Pt(II) (complex 10-Pt), Pd(II) (complex 10-Pd), Ni(II) (complex 10-Ni), Ir(I) (complex 10-Ir), Rh(I) (complex 10-Rh), Au(III) (complex 10-Au), Ag(III) (complex 10-Ag), or Cu(III) (complex 10-Cu),
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 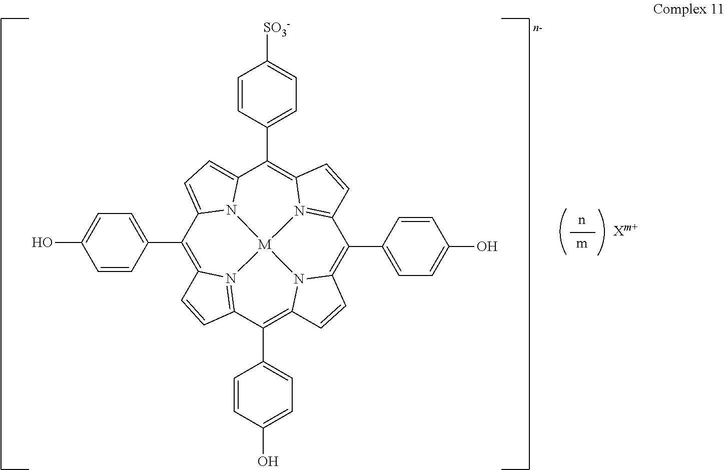
wherein M is Pt(II) (complex 11-Pt), Pd(II) (complex 11-Pd), Ni(II) (complex 11-Ni), Ir(I) (complex 11-Ir), Rh(I) (complex 11-Rh), Au(III) (complex 11-Au), Ag(III) (complex 11-Ag), or Cu(III) (complex 11-Cu),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula; 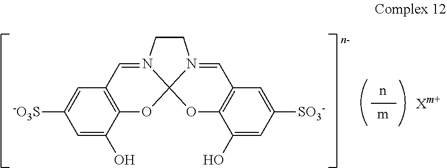
wherein M is Pt(II) (complex 12-Pt), Pd(II) (complex 12-Pd), Ni(II) (complex 12-Ni), Ir(I) (complex 12-Ir), Rh(I) (complex 12-Rh), Au(III) (complex 12-Au), Ag(III) (complex 12-Ag), or Cu(III) (complex 12-Cu);
wherein n− is the number of negative charges carried by the metal complex in the formula, wherein n is a positive integer,
wherein X^{m+} is a counterion to maintain charge neutrality, wherein m is a positive integer, m=n or m≠n,
wherein
$$\frac{n}{m}$$
is the stoichiometry of the counterion in the formula; 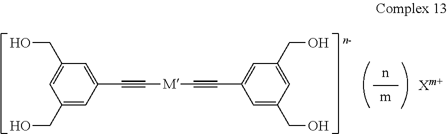
wherein M′ is Ni(0) (complex 13-Ni), Pd(0) (complex 13-Pd), Pt(0) (complex 13-Pt), Cu(I) (complex 13-Cu), Ag(I) (complex 13-Ag), Au(I) (complex 13-Au), Zn(II) (complex 13-Zn), Cd(II) (complex 13-Cd), or Hg(II) (complex 13-Hg),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula; 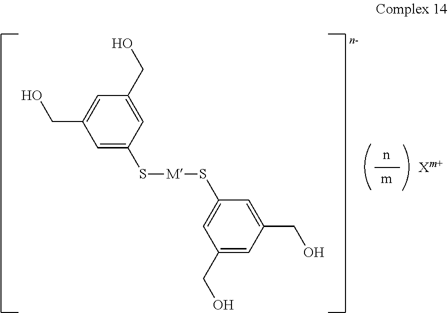
wherein M′ is Ni(O) (complex 14-Ni), Pd(O) (complex 14-Pd), Pt(O) (complex 14-Pt), Cu(I) (complex 14-Cu), Ag(I) (complex 14-Ag), Au(I) (complex 14-Au), Zn(II) (complex 14-Zn), Cd(II) (complex 14-Cd), or Hg(II) (complex 14-Hg),
wherein n− is the number of negative charges carried by the metal complex in the formula, wherein n is zero or a positive integer,
wherein X^{m+} is a counterion to maintain charge neutrality, wherein m is zero or a positive integer, m=n or m≠n,
wherein
$$\frac{n}{m}$$
is the stoichiometry of the counterion in the formula.

13. The compound of claim 1, wherein the analyte is selected from (1) amyloid, plaque, or both, of a protein or peptide and (2) RNA, nucleolus or both.

14. A method for detecting an analyte in a sample, comprising:
(a) combining the compound of claim 1 with the sample,
(b) detecting changes in the photophysical properties of the metal complex,
wherein detection of changes in the photophysical properties of the metal complex indicates the presence of aggregation and supramolecular self-assembly of the metal complex, wherein the presence of aggregation and supramolecular self-assembly of the metal complex indicates the presence of the analyte in the sample.

15. The method of claim 14, wherein the analyte is selected from (1) amyloid, plaque, or both, of a protein or peptide and (2) RNA, nucleolus or both.

16. The method of claim 14, wherein the sample comprises a human or non-human animal bodily fluid, a human or non-human animal tissue, or a combination thereof.

17. The method of claim 16, wherein the bodily fluid is cerebrospinal fluid.

18. The method of claim 16, wherein the tissue is brain tissue.

19. The method of claim 14, wherein analyte is the amyloid, plaque, or both, of a protein or peptide, wherein the amyloid, plaque, or both, of the protein or peptide in the sample comprises thread-like aggregates of the protein or peptide, which are ordered in a β-sheet conformation.

20. A method for testing the efficacy of an inhibitor against amyloidosis and/or fibrillar growth of a protein or peptide, comprising:
(a) combining the compound of claim 1 with an inhibitor-treated sample containing the protein or peptide and, separately, with an untreated sample containing the protein or peptide,
(b) comparing the photophysical properties of the metal complex between the two samples,
wherein the magnitude of the difference in the photophysical properties of the metal complex between the two samples indicates the extent of change in the state of aggregation and supramolecular self-assembly of the metal complex, wherein the extent of change in the state of aggregation and supramolecular self-assembly of the metal complex indicates the efficacy of the inhibitor.

21. A method for imaging an analyte in a sample, comprising:
(a) combining the compound of claim 1 with the sample under conditions to allow for binding of the metal complex of the compound with the analyte and subsequent aggregation and supramolecular self-assembly of the metal complex, wherein aggregation and supramolecular self-assembly of the metal complex generates changes in the photophysical properties of the metal complex,
(b) imaging the analyte based on one or more photophysical properties that are specific for the metal complex after aggregation and supramolecular self-assembly.

22. The method of claim 21, wherein the analyte is selected from (1) amyloid, plaque, or both, of a protein or peptide and (2) RNA, nucleolus or both.

23. The method of claim 21, wherein the sample contains eukaryotic cells optionally selected from the groups consisting of 3T3 cells, A549 cells, Chinese hamster ovary (CHO) cells, HEK 293 cells, HeLa cells, Hep G2 cells, and HT1080 cells.

24. A kit for use in detecting and/or imaging an analyte, comprising, in one or more containers, one or more compounds of claim 1 and optionally instructions for use.

25. The kit of claim 24, wherein the analyte is selected from (1) amyloid, plaque, or both, of a protein or peptide and (2) RNA, nucleolus or both.

26. The kit of claim 24, further comprising a carrier.

27. The kit of claim 24, wherein the presence of the analyte can induce aggregation and supramolecular self-assembly of the metal complex thereon after binding, wherein the aggregation and supramolecular self-assembly of the metal complex can be detected by changes in the photophysical properties of the metal complex.
